(19) United States
(12) Patent Application Publication
(10) Pub. No.: US 2021/0163940 A1
(43) Pub. Date: Jun. 3, 2021

(54) COMPOSITIONS AND METHODS FOR NICKING TARGET DNA SEQUENCES

(71) Applicant: The Board of Trustees of the Leland Stanford Junior University, Stanford, CA (US)

(72) Inventors: Xu Hua FU, Stanford, CA (US); Andrew FIRE, Stanford, CA (US); Justin D. SMITH, Stanford, CA (US)

(73) Assignee: The Board of Trustees of the Leland Stanford Junior University, Stanford, CA (US)

(21) Appl. No.: 17/083,918

(22) Filed: Oct. 29, 2020

Related U.S. Application Data
(63) Continuation of application No. PCT/US2019/030452, filed on May 2, 2019.
(60) Provisional application No. 62/666,612, filed on May 3, 2018.
(60) Provisional application No. 62/719,255, filed on Aug. 17, 2018.

(51) Int. Cl. C12N 15/113; C12N 9/22

(52) U.S. Cl. CPC C12N 15/113; C12N 2310/20; C12N 9/22

(57) ABSTRACT

The disclosure describes that both Cas9 and Cpf1 enzymes can exhibit potent nickase activities on an extensive class of mismatched dsDNA targets. These properties allow the production of efficient nickases for a chosen dsDNA target sequence, without modification of the nuclease protein, using guide RNAs with a variety of patterns of mismatch to the intended DNA target. In parallel to the nicking activities observed with purified Cas9 in vitro, sequence-dependent nicking for both perfectly matched and partially mismatched target sequences in a Saccharomyces cerevisae in vivo system was observed.

### CROSS-REFERENCE TO RELATED APPLICATIONS

This application is a continuation of International Application No. PCT/US2019/030452, filed May 2, 2019, which claims priority to U.S. Provisional Application No. 62/666,612, filed May 3, 2018, and U.S. Provisional Application No. 62/719,255, filed Aug. 17, 2018, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under contract NSF-GFRP to Xu Hua Fu awarded by the National Science Foundation and under contract GM037706 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Sep. 7, 2018, is named 079445-1213315-001720US_SL.txt and is 127,683 bytes in size.

### BACKGROUND OF THE INVENTION

The development of genome editing using nucleases is the foundation for utilizing gene correction as a viable therapeutic strategy for both genetic and non-genetic diseases (Naldini, Nature Reviews. Genetics, 12, 301-315 (2011)).

### BRIEF SUMMARY OF THE INVENTION

In a first aspect, provided herein is a dual nickase CRISPR system comprising: (a) a first CRISPR-Cas nickase comprising a first CRISPR-Cas nuclease and a first guide RNA (gRNA) comprising a first targeting region capable of guiding the first nuclease to cleave one strand of a double-stranded target DNA sequence, wherein the first gRNA comprises at least one nucleotide mismatch or deletion in the first targeting region relative to the target DNA sequence; and (b) a second CRISPR-Cas nickase comprising a second CRISPR-Cas nuclease and a second gRNA comprising a second targeting region capable of guiding the second nuclease to cleave the complementary strand of the target DNA sequence, wherein the second gRNA comprises at least one nucleotide mismatch or deletion in the second targeting region relative to the target DNA sequence.

In some embodiments of the first aspect, the first CRISPR-Cas nuclease and/or the second CRISPR-Cas nuclease is a wild-type CRISPR-Cas nuclease. In some embodiments, the first CRISPR-Cas nuclease and/or the second CRISPR-Cas nuclease is a mutant CRISPR-Cas nuclease.

In another aspect, provided herein is a composition comprising a first RNA-guided DNA nuclease, a first guide RNA (gRNA), a second RNA-guided DNA nuclease, and a second gRNA, wherein the first gRNA comprises at least one nucleotide mismatch or deletion relative to a region of a first strand of a target DNA, wherein the second gRNA comprises at least one nucleotide mismatch or deletion relative to a region of a second strand of the target DNA, wherein the first gRNA guides the first nuclease to bind to and cleave in the region of the first strand of the target DNA, wherein the second gRNA guides the second nuclease to bind to and cleave in the region of the second strand of the target DNA, and wherein cleavage of the first and second strands of the target DNA by the first and second nucleases produces a double-strand break in the target DNA.

In some embodiments of the previous aspect, the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA. In some embodiments, the at least one nucleotide mismatch relative to the region of the first strand of the target DNA is a transversion point mutation and/or wherein the at least one nucleotide mismatch relative to the region of the second strand of the target DNA is a transversion point mutation.

In certain embodiments, the first gRNA comprises a nucleotide deletion in a seed region and a nucleotide mismatch in a distal region of the of the first gRNA relative to the region of the first strand of the target DNA, and wherein the second gRNA comprises a nucleotide deletion in a seed region and a nucleotide mismatch in a distal region of the second gRNA relative to the region of the second strand of the target DNA.

In certain embodiments, the first gRNA comprises a nucleotide mismatch that is a transversion point mutation in a seed region and a nucleotide mismatch that is a transversion point mutation in a distal region of the of the first gRNA relative to the region of the first strand of the target DNA, and wherein the second gRNA comprises two nucleotide mismatches that are transversion point mutations in a distal region of the second gRNA relative to the region of the second strand of the target DNA.

In some embodiments of this aspect, the first RNA-guided DNA nuclease and/or the second RNA-guided DNA nuclease is a wild-type CRISPR-Cas nuclease. In some embodiments, the first RNA-guided DNA nuclease and/or the second RNA-guided DNA nuclease is a mutant CRISPR-Cas nuclease.

In another aspect, provided herein is a nickase CRISPR system comprising: a CRISPR-Cas nickase comprising a CRISPR-Cas nuclease and a guide RNA (gRNA) comprising a targeting region capable of guiding the nuclease to cleave one strand of a double-stranded target DNA sequence, wherein the gRNA comprises at least one nucleotide mismatch or deletion in the targeting region relative to the target DNA sequence. In some embodiments, the CRISPR-Cas nuclease is a wild-type CRISPR-Cas nuclease. In some embodiments, the CRISPR-Cas nuclease is a mutant CRISPR-Cas nuclease.

In another aspect, provided herein is a composition comprising a RNA-guided DNA nuclease and a guide RNA (gRNA), wherein the gRNA comprises at least one nucleotide mismatch or deletion relative to a region in a target DNA, wherein the gRNA guides the nuclease to bind to and cleave in the region in the target DNA, and wherein cleavage of the target DNA by the nuclease produces a single-strand break in the target DNA.

In some embodiments of this aspect, the at least one nucleotide mismatch or deletion relative to the region in the target DNA is in a distal region of the gRNA. In some embodiments, the at least one nucleotide mismatch relative to the region in the target DNA is a transversion point mutation. In some embodiments, the gRNA comprises a nucleotide deletion in a seed region and a nucleotide mismatch in a distal region of the of the gRNA relative to the region in the target DNA. In some embodiments, the gRNA comprises a nucleotide mismatch that is a transversion point mutation in a seed region and a nucleotide mismatch that is a transversion point mutation in a distal region of the of the gRNA relative to the region in the target DNA.

In some embodiments of this aspect, the RNA-guided DNA nuclease is a wild-type CRISPR-Cas nuclease. In some embodiments, the RNA-guided DNA nuclease is a mutant CRISPR-Cas nuclease.

In another aspect, provided herein is also a method of cleaving both strands of a target DNA, comprising providing a first RNA-guided DNA nickase, a first gRNA, a second RNA-guided DNA nickase, and a second gRNA, wherein the first gRNA comprises at least one nucleotide mismatch to a region of a first strand in the target DNA, the second gRNA comprises at least one nucleotide mismatch to a region of a second strand in the target DNA, the first gRNA guides the first RNA-guided DNA nickase to bind to and cleave in the region of the first strand of the target DNA, the second gRNA guides the second RNA-guided DNA nickase to bind to and cleave in the region of the second strand of the target DNA, and cleavage of both strands by the first and second RNA-guided DNA nickases produces two product DNAs each having an overhang of at least one nucleotide (e.g., at least two, three, four, five, six, seven, eight, nine, ten, eleven, twelve, thirteen, fourteen, or fifteen nucleotides). In some embodiments of this aspect, the first RNA-guided DNA nickase and/or the second RNA-guided DNA nickase is a wild-type RNA-guided DNA nickase. In some embodiments, the first RNA-guided DNA nickase and/or the second RNA-guided DNA nickase is a mutant RNA-guided DNA nickase. In some embodiments, the method comprises in vivo cleavage of both strands of the target DNA.

In another aspect, provided herein is also a method of cleaving both strands of a target DNA, comprising providing an RNA-guided DNA nickase, a first gRNA, and a second gRNA, wherein the first gRNA comprises at least one nucleotide mismatch to a region of a first strand in the target DNA, the second gRNA comprises at least one nucleotide mismatch to a region of a second strand in the target DNA, the first gRNA guides the RNA-guided DNA nickase to bind to and cleave in the region of the first strand of the target DNA, the second gRNA guides the RNA-guided DNA nickase to bind to and cleave in the region of the second strand of the target DNA, and cleavage of both strands by the RNA-guided DNA nickase produces two product DNAs each having an overhang of at least one nucleotide (e.g., at least two, three, four, five, six, seven, eight, nine, ten, eleven, twelve, thirteen, fourteen, or fifteen nucleotides). In some embodiments, the method comprises in vivo cleavage of both strands of the target DNA.

In some embodiments of the previous aspect, the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA. In particular embodiments, the at least one nucleotide mismatch relative to the region of the first strand of the target DNA is a transversion point mutation and/or wherein the at least one nucleotide mismatch relative to the region of the second strand of the target DNA is a transversion point mutation.

In some embodiments, the first gRNA comprises a nucleotide deletion in a seed region and a nucleotide mismatch in a distal region of the of the first gRNA relative to the region of the first strand of the target DNA, and wherein the second gRNA comprises a nucleotide deletion in a seed region and a nucleotide mismatch in a distal region of the second gRNA relative to the region of the second strand of the target DNA.

In some embodiments, the first gRNA comprises a nucleotide mismatch that is a transversion point mutation in a seed region and a nucleotide mismatch that is a transversion point mutation in a distal region of the of the first gRNA relative to the region of the first strand of the target DNA, and wherein the second gRNA comprises two nucleotide mismatches that are transversion point mutations in a distal region of the second gRNA relative to the region of the second strand of the target DNA.

In some embodiments of this aspect, the RNA-guided DNA nickase is a wild-type RNA-guided DNA nickase. In some embodiments, the RNA-guided DNA nickase is a mutant RNA-guided DNA nickase.

In yet another aspect, provided herein is a method of directing an RNA-guided DNA nickase to a region in a target DNA, comprising contacting the target DNA with the RNA-guided DNA nickase and a gRNA, wherein the gRNA comprises at least one nucleotide mismatch to the region in the target DNA and the gRNA guides the RNA-guided DNA nickase to bind to and cleave in the region in the target DNA. In certain embodiments of this aspect, the RNA-guided DNA nickase is immobilized on the target DNA. In some embodiments, the target DNA is contacted with the RNA-guided DNA nickase and the gRNA in vivo.

Other objects, features, and advantages of the present invention will be apparent to one of skill in the art from the following detailed description and figures.

### BRIEF DESCRIPTION OF THE DRAWINGS

FIGS. 1A and 1B depict the nicking and cleavage in vitro high throughput assay. FIG. 1A depicts the in vitro high throughput assay. The variant circular plasmid library is incubated with CRISPR-Cas nuclease. The plasmid will either linearize (cleave) or remain circular (nicked or uncleaved). Method 1 is used to access cleavage and requires the linearization of the library with a restriction enzyme (EcoRV). Method 2 can be used to access cleavage and nicking and requires no linearization of the variant plasmid library. Whether Method 1 or 2 is selected, the library then undergoes amplification of the target variants and sequenced using high throughput sequencing (Miseq). Finally, the results from high throughput sequencing are used to calculate retention scores for each variant in the library.

FIG. 1B depicts target sequences that were synthesized for the Cpf1 and Cas9 variant libraries. The regions labeled (1) indicates the PAM, the region labeled (4) indicates seed region (positions 1-10), the region labeled (2) indicates the distal region (positions 11-20), and the region labeled (3) indicates the barcode region. For each target, the following variants were synthesized: wild-type, single variants, single deletions, and double consecutive transversion variants.

FIGS. 2A-2C show cleavage assessment of LbCpf1 with the EGFP-1 target for single base transversions (normalized to rol-6) (FIG. 2A), single deletion variants (normalized to whole library) (FIG. 2B), and a subset of double consecutive transversion variants (normalized to whole library) (FIG. 2C). These assays were performed using Method 1 shown in FIG. 1A—linearized substrates. (AF_SOL_820).

FIGS. 3A-3C show nicking/cleavage assessment of LbCpf1 with the EGFP-1 target for single base transversions (normalized to rol-6) (FIG. 3A), single deletion variants (normalized to whole library) (FIG. 3B), and a subset of double consecutive transversion variants (normalized to whole library) (FIG. 3C). These assays were performed using Method 2 shown in FIG. 1A—circular substrates. (AF_SOL_805).

FIGS. 4A-4C show mismatches in target DNA promote nicking by LbCpf1. FIG. 4A shows a nicking gel assay with LbCpf1 WT EGFP-1 target and gRNA (left) and mutated EGFP-1 target (p703) with WT EGFP-1 gRNA (right). WT EGFP-1 and gRNA nicks and linearizes the plasmid. The mutated DNA target preferentially nicks the plasmid. FIG. 4B shows a nicking gel assay with mutated EGFP-1 target (p705) with WT EGFP-1 gRNA. Preferential nicking with some linearization is observed for the mutated EGFP-1 target (p705). FIG. 4C shows a nicking gel assay with WT unc-22A gRNA (u22) with WT EGFP-1(p658) and unc-22A (p648) target and unc-22A mismatched gRNA with WT EGFP-1(p658) and unc-22A (p648) target. The u22 gRNA linearizes the WT unc-22A (p648) target while having no effect on the EGFP-1 (p658) target. The mismatched unc-22A gRNA promotes nicking when paired with the WT unc-22A (p648) target and not with EGFP-1 (p658). Nicking is shown to be RNA-guided and specific.

FIGS. 5A and 5B show mismatches in target DNA promote nicking by Cas9. FIG. 5A shows WT unc-22A gRNA and mutated unc-22A target (left) and WT unc-22A gRNA and WT unc-22A target (right). Cas9 efficiently nicks plasmids. FIG. 5B shows WT EGFP-2 gRNA with mutated EGFP-2 DNA targets (p775 and p777) and WT EGFP-2 with WT EGFP-2 DNA (middle). Both mutated EGFP-2 targets are nicked efficiently. The p775 mutation if given enough time eventually linearizes the plasmid (left). The p777 mutation remains nicked (right).

FIG. 6A shows the LbCpf1 results for EGFP-2 (Method 1-Linearized library, AF_SOL_821; Method 2-Circular library, AF_SOL_805).

FIG. 6B shows LbCpf1 results for double consecutive transversion effects in EGFP-2.

FIG. 6C shows LbCpf1 results for single deletion effects in EGFP-2.

FIG. 6D shows the LbCpf1 results for rol-6 (Method 1-Linearized library; Method 2-Circular library, AF_SOL_805).

FIG. 6E shows LbCpf1 results for single deletion effects in rol-6.

FIG. 6F shows LbCpf1 results for double consecutive transversion effects in rol-6.

FIG. 7A shows the AsCpf1 results for EGFP-1 (Method 1-Linearized library, AF_SOL_820; Method 2-Circular library, AF_SOL_809).

FIG. 7B shows LbCpf1 results for single deletion effects in EGFP-1.

FIG. 7C shows LbCpf1 results for double consecutive transversion effects in EGFP-1.

FIG. 7D shows the AsCpf1 results for EGFP-2 (Method 1-Linearized library, AF_SOL_821; Method 2-Circular library, AF_SOL_810).

FIG. 7E shows AsCpf1 results for single deletion effects in EGFP-1.

FIG. 7F shows AsCpf1 results for double consecutive transversion effects in EGFP-1.

FIG. 7G shows the AsCpf1 results for unc-22A (Method 1-Linearized library, AF_SOL_821; Method 2-Circular library, AF_SOL_810).

FIG. 7H shows AsCpf1 results for single deletion effects in unc-22A.

FIG. 7I shows AsCpf1 results for double consecutive transversion effects in unc-22A.

FIG. 7J shows the AsCpf1 results for rol-6 (Method 1-Linearized library, AF_SOL_821; Method 2-Circular library, AF_SOL_810).

FIG. 7K shows AsCpf1 results for single deletion effects in rol-6.

FIG. 7L shows AsCpf1 results for double consecutive transversion effects in rol-6.

FIG. 8A shows the FnCpf1 results for EGFP-1 (Method 1-Linearized library, AF_SOL_820; Method 2-Circular library, AF_SOL_811).

FIG. 8B shows FnCpf1 results for single deletion effects in EGFP-1.

FIG. 8C shows FnCpf1 results for double consecutive transversion effects in EGFP-1.

FIG. 8D shows the FnCpf1 results for EGFP-2 (Method 1-Linearized library, AF_SOL_821; Method 2-Circular library, AF_SOL_811).

FIG. 8E shows FnCpf1 results for single deletion effects in EGFP-2.

FIG. 8F shows FnCpf1 results for double consecutive transversion effects in EGFP-2.

FIG. 8G shows the FnCpf1 results for rol-6 (Method 1-Linearized library, AF_SOL_821; Method 2-Circular library, AF_SOL_811).

FIG. 8H shows FnCpf1 results for single deletion effects in rol-6.

FIG. 8I shows FnCpf1 results for double consecutive transversion effects in rol-6.

FIGS. 9A and 9B are agarose gels showing the results of the AsCpf1 nicking experiments.

FIGS. 9C and 9D are agarose gels showing the results of the FnCpf1 nicking experiments.

FIGS. 9E and 9F are agarose gels showing the results of the LbCpf1 nicking experiments with equivalent mismatches in EGFP-1 gRNA paired with WT EGFP-1 target.

FIGS. 10A-10D are agarose gels showing the analysis of Cpf1 activities on additional target sequences (FIG. 10A: DNMT1; FIGS. 10B and 10C: WTAP exon8; FIG. 10C: Fancf). A series of additional guide/target combinations were tested using individual agarose gel migration assays. These were targets where no high throughput data was available. Instead, general rules from unc-22, gfp1, and rol-6 were used to design potential nicking guides. Sequences of guides and targets were as shown in the figures.

FIG. 11A shows the Cas9 high throughput nicking assay results for EGFP-1 (Method 2-Circular Library, AF_SOL_854).

FIG. 11B shows Cas9 nicking assay results with single deletions for EGFP-1.

FIG. 11C shows Cas9 nicking assay results with double consecutive transversions for EGFP-1.

FIG. 11D shows the Cas9 high throughput nicking assay results for EGFP-2 (Method 2-Circular Library, AF_SOL_854).

FIG. 11E shows Cas9 nicking assay results with single deletions for EGFP-2.

FIG. 11F shows Cas9 nicking assay results with double consecutive transversions for EGFP-2.

FIG. 11G shows the Cas9 high throughput nicking assay results for rol-6 (Method 2-Circular Library, AF_SOL_854).

FIG. 11H shows Cas9 nicking assay results with single deletions for rol-6.

FIG. 11I shows Cas9 nicking assay results with double consecutive transversions for rol-6.

FIG. 11J shows the Cas9 high throughput nicking assay results for unc-22a (Method 2-Circular Library, AF_SOL_854).

FIG. 11K shows Cas9 nicking assay results with single deletions for unc-22A.

FIG. 11L shows Cas9 nicking assay results with double consecutive transversions for unc-22A.

FIG. 12 shows a schematic drawing of a Type I library. Type I libraries were created using oligo pooled array synthesis. Type II libraries were made using degenerate oligo synthesis.

FIG. 13 shows the double mutant cleavage and nicking specificity profile with Cas9 and the variant library generated via degenerate oligonucleotide synthesis. White boxes indicate no mutant available. The single box labeled “NoMutant” indicates what retention of the WT unc-22A sequence. The diagonal indicates the retention of the single mutant at that position (Method 2-Circular Library. AF_SOL_827).

FIGS. 14A-14D are agarose gels showing the results of the Cas9 nicking experiments. FIG. 14A: unc-22A target with mismatched unc-22A gRNA; FIG. 14B: EGFP-2 target with mismatched EGFP-2 gRNAs; FIG. 14C: variant unc-22A target with EGFP-2 gRNA; and FIG. 14D: rol-6 target with unc-22A gRNA.

FIG. 15A shows the Cas9 D10A specificity profile results for single base transversion effects (Method 2-Circular Library, AF_SOL_855).).

FIGS. 15B and 15C show the Cas9 D10A specificity profile results for single deletion and double consecutive transversion variants.

FIG. 16A shows, in the context of Cas9- and target-match-dependent nicking of precise and imprecise targets by Cas9 in vivo, reproducible nicking signals for matched targets following Cas9 induction in yeast. This plot compares differential retention [log 2(circular_assay_retention)−log 2(linear_assay_retention)] for induced libraries from two different but functionally equivalent yeast strains, BY4741 and ΔKU70. Individual dots represent different target sequences, each assessed as a median over multiple barcoded instances in each library. Targets unrelated to unc-22A (grey hollow circles; “Protospacer 4” variants²⁶) and with perfect unc-22A match in the gRNA homology but mismatches in the GG pam (blue squares) show no substantial differential retention in either sample. Perfectly matched targets with canonical “NGG” PAM sites (black circle) show substantial differential retention in the two yeast populations. Targets with single mismatch (red dots) show a spectrum of different retention differentials, ranging from no difference to differences comparable to the perfect target match. The two yeast populations give highly similar results with calculated sample-to-sample correlations between the two yeast populations of 0.81 (Pearson; two-tailed p-value=9.6E-17), and 0.76 (Spearman; two-tailed p-value=1.6E-13).

FIGS. 16B-16E show, in the context of Cas9- and target-match-dependent nicking of precise and imprecise targets by Cas9 in vivo, similar sequence requirements for nicked substrate accumulation under diverse in vitro and in vivo conditions. Top graphs show circular and linear assay retention scores for yeast (in vivo) experiments in the BY4741 and AKU70 genetic backgrounds. Each plot shows median retention for multiple (barcoded) transversion variants at each position (averaged where duplicate sample are available for the relevant conditions). Retentions are calculated using the unrelated PS4 sequence as an internal standard, with initial library abundance obtained from measurement of target species incidence in parallel libraries with yeast that have not been induced. Below are equivalent retention score plots for in vitro analysis in which an equivalent library of targets was interacted with purified Cas9 in one of two buffer conditions (a relatively active “Thermo-Pol” buffer condition and a less active “Cas9 buffer” condition; see methods for buffer details).

FIG. 16F shows, in the context of Cas9- and target-match-dependent nicking of precise and imprecise targets by Cas9 in vivo, differential retention comparison between in vitro and in vivo samples. The in vivo sample shown here is from BY4741 (at 2.5 generations) compared to the initial rate (1 minute) of nicking in vitro (Cas9 buffer). Correlations are Pearson: 0.54, two-tailed p-value=3.6E-6 and Spearman 0.49, two-tailed p-value=3.2E-5. Each point on the plot represents a single gRNA homology+PAM sequence class, showing a median of differential retentions derived from independent flanking-barcoded instances of each sequence.

### DETAILED DESCRIPTION OF THE INVENTION

### I. Introduction

Bacteria and archaea are constantly challenged by invasive genetic elements (e.g., bacteriophage, transposons, and plasmids). To combat these threats, prokaryotes evolved an adaptive immune system known as CRISPR-Cas (clustered regularly interspaced short palindromic repeats and CRISPR-associated proteins)¹⁻⁴. This immune system is able to capture foreign genetic elements into repeats in the CRISPR loci as short DNA segments. These captured “spacer” sequences are expressed in the context of precursor CRISPR RNAs (pre-crRNAs), which are processed into small CRISPR RNAs (referred to as guide RNAs or gRNAs)¹⁻⁴. CRISPR-Cas proteins use the gRNA complementarity and sometimes a protospacer adjacent motif (PAM) to recognize and cleave the target thus conferring immunity to the invading elements¹⁻⁴. The CRISPR-Cas systems have been adapted as versatile genome editing tools that are ubiquitously used in many disciplines⁵.

Streptococcus pyogenes Cas9 is a type II-A CRISPR-Cas nuclease that is widely studied and used in genome editing and epigenome manipulation^{6,7}. Cas9 is a blunt cutting nuclease that can target specific DNA sequences using a NGG PAM and a gRNA⁸. Many variants of Cas9 have been developed (e.g., transcription activating/repressing, nicking, nuclease-dead, etc.)^{9,10}. One set of observations of particular note in reference to such variants are experiments in which nicking of targets can be used to guide homology-dependent gene editing, with apparent advantages in specificity¹¹ and efficiency¹² over double-stranded break strategies.

Cpf1 is a minimal type V CRISPR-Cas nuclease that uses a single Cas endonuclease paired with a gRNA to cleave complementary DNA targets¹³⁻¹⁶. Cpf1 homologs from three species have been successfully adapted for genome editing: Acidaminococcus sp. (AsCpf1), Francisella novicida (FnCpf1), and Lachnospiraceae bacterium (LbCpf1)¹⁴. Unlike Cas9, Cpf1 recognizes a T-rich PAM (TTTN/TTTV), theoretically opening a wide array of sites not available for Cas9 editing^{16,17}. Cpf1 processes its own pre-CRISPR RNA, allowing this to be a potential platform for multi-gene functional analysis^{16,17}. Despite numerous possible advantages, Cpf1 homologs have yet to be used as extensively in genome editing applications as Cas9.

This work began with an unexpected observation that certain sequences were enriched in assays that preferentially capture nicked targets in a population of diverse molecules. The disclosure features the observation of efficient nicking activities of Cpf1 and Cas9 nucleases on specific classes of mismatched DNA targets.

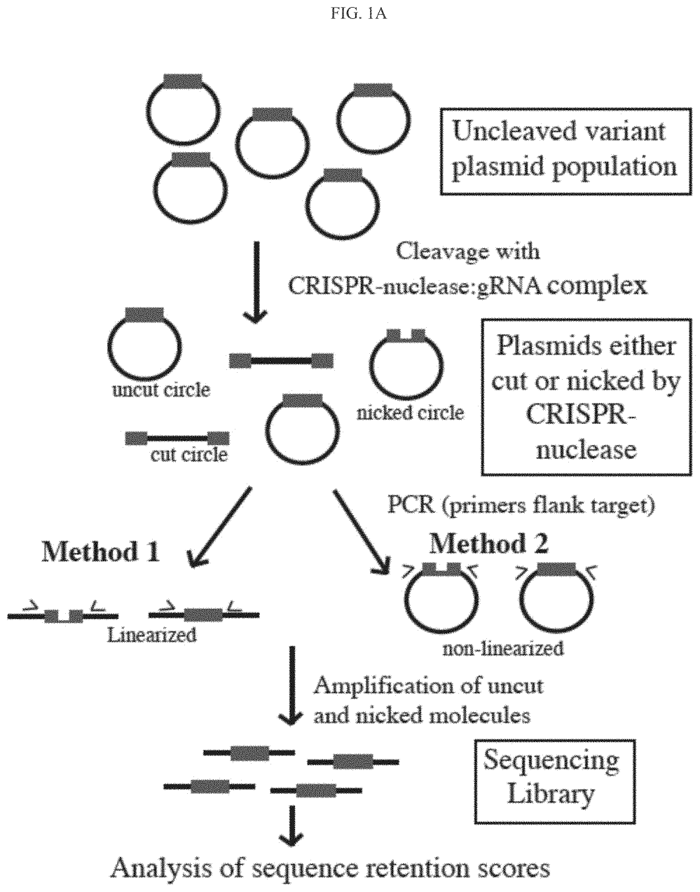

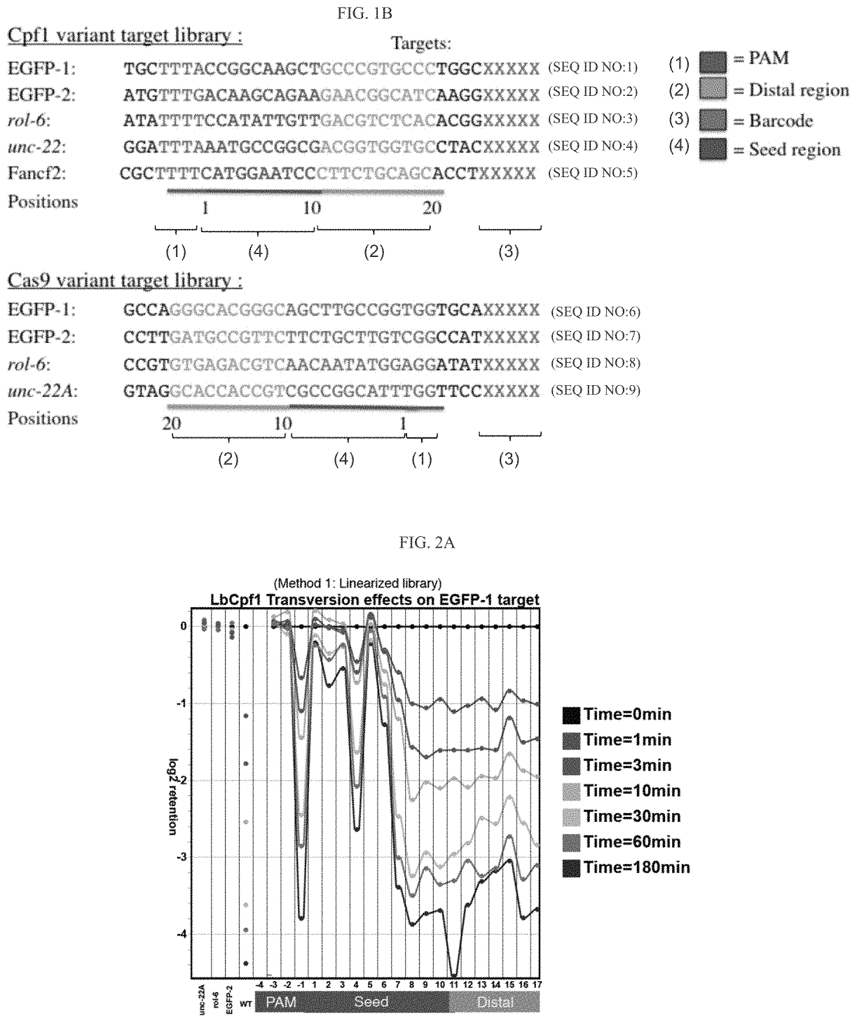

Sequence dependence for CRISPR-Cas nuclease activity was first addressed by building variant libraries of plasmids with a diversity of perfectly matched (“wild-type”) and mismatched (“mutant”) target sequences. Each sequence (wild-type or mutant) is represented by several barcoded species. The variant library was challenged with CRISPR-Cas nuclease protein programmed with single gRNA, with sequencing used to determine which templates remained following nuclease treatment. Additional information on the nature of the cleaved substrates comes from assays with and without a whole-library (backbone) linearization step after CRISPR-Cas nuclease cleavage of substrates and before PCR (FIG. 1A). In particular, backbone-intact assays provides a potential to distinguish nicked from closed circles due to the higher PCR yields observed from molecules where a nicking event has allowed the two plasmid strands to become topologically unlinked¹⁸.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “nuclease” or “Cas nuclease” refers to a Clustered Regularly Interspaced Short Palindromic Repeats-associated polypeptide or nuclease that nicks one strand or cleaves both strands of a target DNA sequence at sites specified by a nucleotide guide sequence contained within a crRNA transcript. A Cas nuclease requires both a crRNA and a tracrRNA for site-specific DNA recognition and cleavage. The crRNA associates, through a region of partial complementarity, with the tracrRNA to guide the Cas nuclease to a region homologous to the crRNA in the target DNA called a “protospacer.” A Cas nuclease may be a wild-type Cas nuclease or a mutant Cas nuclease.

The term “nickase” refers to a nuclease which cleaves only a single strand of a double-stranded DNA. A nickase may be a wild-type nickase or a mutant nickase.

The term “CRISPR-Cas nickase” as referred to herein comprises a CRISPR-Cas nuclease (e.g., a wild-type or mutant CRISPR-Cas nuclease) and a guide RNA (gRNA) which comprises at least one nucleotide mismatch or deletion in a targeting region of the gRNA relative to a double-stranded target DNA sequence and functions to guide the nuclease to cleave one strand of the target DNA.

The term “mutant” Cas nuclease or nickase refers to a nuclease or nickase that has at least one amino acid substitution, addition, or deletion relative to a wild-type nuclease or nickase (e.g., a wild-type Cpf1 (also known as Cas12a) or a wild-type Cas9). A mutant Cas nuclease or nickase still maintains the nicking or cleavage activity. In some embodiments, a mutant Cas nuclease or nickase has at least 70% sequence identity (e.g., 75%, 80%, 85%, 90%, 91%, 92%, 93%, 94%, 95%, 96%, 97%, 98%, or 99% sequence identity) to a wild-type nuclease or nickase. For example, a mutant Cas9 nuclease may comprise a D10A mutation and/or a H840A mutation. Other examples of mutations present in a mutant Cas9 nuclease may include, without limitation, N854A and N863A.

The term “single guide RNA” or “gRNA” refer to a DNA-targeting RNA containing a guide sequence that targets the Cas nuclease to the target genomic DNA and a scaffold sequence that interacts with the Cas nuclease (e.g., tracrRNA), and optionally, a donor repair template

The term “distal region” refers to the 3′ terminal region in a gRNA. The distal region is located after the seed region (e.g., 3′ of the seed region), which is located after the protospacer adjacent motif (PAM) portion (e.g., 3′ of the PAM) in the gRNA. In some embodiments, a distal region in a gRNA may be from approximately position 11 (e.g., position 10, 11, or 12) to approximately position 20 (e.g., position 19, 20, or 21) in the gRNA (with position 1 being the start of the seed region).

The term “seed region” refers to the region in a gRNA that is between the PAM portion of the gRNA and the distal region of the gRNA. In some embodiments, the seed region may comprise about 7 to about 11 (e.g., 7, 8, 9, 10, or 11) nucleotides.

The term “transverse point mutation” refers the substitution of a purine (a two-ring nucleotide) for a pyrimidine (a one-ring nucleotide) or vice versa. For example, the substitution of an adenosine for a cytosine or vice versa is a transverse point mutation. The substitution of a guanosine for a thymidine or vice versa is a transverse point mutation.

The term “percent (%) identity” as used herein refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

Unless defined otherwise, all technical and scientific terms used herein have the same meanings as commonly understood by one of ordinary skill in the art to which this technology belongs. Although exemplary methods, devices and materials are described herein, any methods and materials similar or equivalent to those expressly described herein can be used in the practice or testing of the present technology. For example, the reagents described herein are merely exemplary and that equivalents of such are known in the art. The practice of the present technology can employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Miller and Calos eds. (1987) Gene Transfer Vectors for Mammalian Cells (Cold Spring Harbor Laboratory); and Makrides ed. (2003) Gene Transfer and Expression in Mammalian Cells (Cold Spring Harbor Laboratory).

### III. Nuclease-Mediated Genome Editing

The compositions and methods of the present invention are directed to the differential guide RNA sequence requirements of CRISPR-Cas nucleases (e.g., Cpf1 (also known as Cas12a) and Cas9) for cleavage of the two strands of a target DNA. As a consequence of the differential guide requirements, CRISPR-Cas nucleases can exhibit potent nickase activities on an extensive class of mismatched double-stranded DNA (dsDNA) targets. These properties allow the production of efficient nickases for a chosen dsDNA target sequence, without modification of the nuclease protein, using guide RNAs with a variety of patterns of mismatch to the intended dsDNA target.

In some embodiments, a nucleotide sequence encoding the CRISPR-Cas nuclease (e.g., Cpf1 (also known as Cas12a) and Cas9) is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 polypeptide are commercially available from, e.g., Addgene, Life Technologies, Sigma-Aldrich, and Origene.

In other embodiments, a nucleotide sequence encoding the CRISPR-Cas nuclease (e.g., Cpf1 (also known as Cas12a) and Cas9) is present as an RNA (e.g., mRNA). The RNA can be produced by any method known to one of ordinary skill in the art. As non-limiting examples, the RNA can be chemically synthesized or transcribed in vitro. In certain embodiments, the RNA comprises an mRNA encoding a Cas nuclease such as a Cas9 polypeptide, a Cpf1 polypeptide, or a variant thereof. For example, the Cas9 mRNA can be generated through in vitro transcription of a template DNA sequence such as a linearized plasmid containing a Cas9 open reading frame (ORF). The Cas9 ORF can be codon optimized for expression in mammalian systems. In some instances, the Cas9 mRNA encodes a Cas9 polypeptide with an N- and/or C-terminal nuclear localization signal (NLS). In other instances, the Cas9 mRNA encodes a C-terminal HA epitope tag. In yet other instances, the Cas9 mRNA is capped, polyadenylated, and/or modified with 5-methylcytidine. Cas9 mRNA is commercially available from, e.g., TriLink BioTechnologies, Sigma-Aldrich, and Thermo Fisher Scientific.

In yet other embodiments, the CRISPR-Cas nuclease (e.g., Cpf1 (also known as Cas12a) and Cas9) is present as a polypeptide. The polypeptide can be produced by any method known to one of ordinary skill in the art. As non-limiting examples, the polypeptide can be chemically synthesized or in vitro translated. In certain embodiments, the polypeptide comprises a Cas protein such as a Cas9 protein, a Cpf1 protein, or a variant thereof. For example, the Cas9 protein can be generated through in vitro translation of a Cas9 mRNA described herein. In some instances, the Cas protein such as a Cas9 protein, a Cpf1 protein, or a variant thereof can be complexed with a single guide RNA (gRNA) such as a modified gRNA to form a ribonucleoprotein (RNP). For example, the Cas9 protein is commercially available from, e.g., PNA Bio (Thousand Oaks, Calif., USA) and Life Technologies (Carlsbad, Calif., USA).

CRISPR/Cas System

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by an approximately 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single guide RNA” or “gRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP 269215, and the amino acid sequence of Streptococcus thermophilus wild-type Cas9 polypeptide is set forth, e.g., in NBCI Ref. Seq. No. WP_011681470.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g., the two catalytic domains are derived from different bacteria species.

Useful variants or mutants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. In some embodiments, a double-strand break can be introduced using a Cas9 nickase if at least two gRNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

### IV. Guide RNA (gRNA)

In some embodiments, the methods of the present invention comprise compositions and methods that utilize one or more guide nucleic acids, e.g., guide RNAs (gRNAs). In some embodiments, the compositions and methods described herein utilize two gRNAs. In particular embodiments, the first gRNA comprises a first targeting region capable of guiding a first nuclease to cleave one strand of a double-stranded target DNA sequence and at least one nucleotide mismatch or deletion in the first targeting region relative to the target DNA sequence. In particular embodiments, the second gRNA comprises a second targeting region capable of guiding a second nuclease to cleave the complementary strand of the target DNA sequence and at least one nucleotide mismatch or deletion in the second targeting region relative to the target DNA sequence.

The gRNA may comprise a first nucleotide sequence that is complementary to a specific sequence within a target DNA (e.g., a guide sequence) and a second nucleotide sequence comprising a protein-binding sequence that interacts with a DNA nuclease (e.g., Cas9 nuclease). In some embodiments, the guide sequence of a gRNA may comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end that can direct the DNA nuclease (e.g., Cas9 nuclease) to the target DNA site using RNA-DNA complementarity base pairing. In some embodiments, the guide sequence of a gRNA comprises about 100 nucleic acids at the 5′ end that can direct the DNA nuclease (e.g., Cas9 nuclease) to the target DNA site using RNA-DNA complementarity base pairing. In some embodiments, the guide sequence comprises 20 nucleic acids at the 5′ end that can direct the DNA nuclease (e.g., Cas9 nuclease) to the target DNA site using RNA-DNA complementarity base pairing. In other embodiments, the guide sequence comprises less than 20, e.g., 19, 18, 17, 16, 15 or less, nucleic acids that are complementary to the target DNA site. In some instances, the guide sequence contains at least one nucleic acid mismatch in the complementarity region at the 5′ end of the targeting region. In some instances, the guide sequence contains about 1 to about 10 nucleic acid mismatches in the complementarity region at the 5′ end of the targeting region.

In some embodiments, the protein-binding scaffold sequence of the gRNA can comprise two complementary stretches of nucleotides that hybridize to one another to form a double-stranded RNA duplex (dsRNA duplex). The protein-binding scaffold sequence can be between about 30 nucleic acids to about 200 nucleic acids, e.g., about 40 nucleic acids to about 200 nucleic acids, about 50 nucleic acids to about 200 nucleic acids, about 60 nucleic acids to about 200 nucleic acids, about 70 nucleic acids to about 200 nucleic acids, about 80 nucleic acids to about 200 nucleic acids, about 90 nucleic acids to about 200 nucleic acids, about 100 nucleic acids to about 200 nucleic acids, about 110 nucleic acids to about 200 nucleic acids, about 120 nucleic acids to about 200 nucleic acids, about 130 nucleic acids to about 200 nucleic acids, about 140 nucleic acids to about 200 nucleic acids, about 150 nucleic acids to about 200 nucleic acids, about 160 nucleic acids to about 200 nucleic acids, about 170 nucleic acids to about 200 nucleic acids, about 180 nucleic acids to about 200 nucleic acids, or about 190 nucleic acids to about 200 nucleic acids. In certain aspects, the protein-binding sequence can be between about 30 nucleic acids to about 190 nucleic acids, e.g., about 30 nucleic acids to about 180 nucleic acids, about 30 nucleic acids to about 170 nucleic acids, about 30 nucleic acids to about 160 nucleic acids, about 30 nucleic acids to about 150 nucleic acids, about 30 nucleic acids to about 140 nucleic acids, about 30 nucleic acids to about 130 nucleic acids, about 30 nucleic acids to about 120 nucleic acids, about 30 nucleic acids to about 110 nucleic acids, about 30 nucleic acids to about 100 nucleic acids, about 30 nucleic acids to about 90 nucleic acids, about 30 nucleic acids to about 80 nucleic acids, about 30 nucleic acids to about 70 nucleic acids, about 30 nucleic acids to about 60 nucleic acids, about 30 nucleic acids to about 50 nucleic acids, or about 30 nucleic acids to about 40 nucleic acids.

In some embodiments, the gRNA may be in a truncated form comprising a guide sequence having a shorter region of complementarity to a target DNA sequence (e.g., less than 20 nucleotides in length). In certain instances, the truncated gRNA provides improved DNA nuclease (e.g., Cas9 nuclease) specificity by reducing off-target effects. For example, a truncated gRNA can comprise a guide sequence having 17, 18, or 19 complementary nucleotides to a target DNA sequence (e.g., 17-18, 17-19, or 18-19 complementary nucleotides). See, e.g., Fu et al., Nat. Biotechnol., 32(3): 279-284 (2014).

A gRNA may be selected using a web-based software. As a non-limiting example, considerations for selecting a gRNA can include, e.g., the PAM sequence for the Cas9 nuclease to be used, and strategies for minimizing off-target modifications. Tools, such as the CRISPR Design Tool, can provide sequences for preparing the gRNA, for assessing target modification efficiency, and/or assessing cleavage at off-target sites.

The gRNA can be produced by any method known to one of ordinary skill in the art. In some embodiments, a nucleotide sequence encoding the gRNA is cloned into an expression cassette or an expression vector. In certain embodiments, the nucleotide sequence is produced by PCR and contained in an expression cassette. For instance, the nucleotide sequence encoding the gRNA can be PCR amplified and appended to a promoter sequence, e.g., a U6 RNA polymerase III promoter sequence. In other embodiments, the nucleotide sequence encoding the gRNA is cloned into an expression vector that contains a promoter, e.g., a U6 RNA polymerase III promoter, and a transcriptional control element, enhancer, U6 termination sequence, one or more nuclear localization signals, etc. In some embodiments, the expression vector is multicistronic or bicistronic and can also include a nucleotide sequence encoding a fluorescent protein, an epitope tag and/or an antibiotic resistance marker. In certain instances of the bicistronic expression vector, the first nucleotide sequence encoding, for example, a fluorescent protein, is linked to a second nucleotide sequence encoding, for example, an antibiotic resistance marker using the sequence encoding a self-cleaving peptide, such as a viral 2A peptide. Viral 2A peptides including foot-and-mouth disease virus 2A (F2A); equine rhinitis A virus 2A (E2A); porcine teschovirus-1 2A (P2A) and Thoseaasigna virus 2A (T2A) have high cleavage efficiency such that two proteins can be expressed simultaneously yet separately from the same RNA transcript.

Suitable expression vectors for expressing the gRNA are commercially available from Addgene, Sigma-Aldrich, and Life Technologies. The expression vector can be pLQ1651 (Addgene Catalog No. 51024) which includes the fluorescent protein mCherry. Non-limiting examples of other expression vectors include pX330, pSpCas9, pSpCas9n, pSpCas9-2A-Puro, pSpCas9-2A-GFP, pSpCas9n-2A-Puro, the GeneArt® CRISPR Nuclease OFP vector, the GeneArt® CRISPR Nuclease OFP vector, and the like.

In certain embodiments, the gRNA is chemically synthesized. The gRNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J. American Chemical Society 125, 940-950 (2003).

Modified gRNA

In particular embodiments, the gRNA is chemically modified. As a non-limiting example, the gRNA is a modified gRNA comprising a first nucleotide sequence substantially complementary to a target nucleic acid (e.g., a guide sequence or crRNA) and a second nucleotide sequence that interacts with a Cas polypeptide (e.g., a scaffold sequence or tracrRNA).

Without being bound by any particular theory, gRNAs containing one or more chemical modifications can increase the activity, stability, and specificity and/or decrease the toxicity of the modified gRNA compared to a corresponding unmodified gRNA when used for CRISPR-based genome editing, e.g., homologous recombination. Non-limiting advantages of modified gRNAs include greater ease of delivery into target cells, increased stability, increased duration of activity, and reduced toxicity. The modified gRNAs described herein as part of a CRISPR/Cas9 system may provide higher frequencies of on-target genetic editing (e.g., homologous recombination), improved activity, and/or specificity compared to their unmodified sequence equivalents.

One or more nucleotides of the guide sequence and/or one or more nucleotides of the scaffold sequence can be a modified nucleotide. For instance, a guide sequence that is about 20 nucleotides in length may have 1 or more, e.g., 1, 2, 3, 4, 5, 6, 7, 8, 9, 10, 11, 12, 13, 14, 15, 16, 17, 18, 19, or 20 modified nucleotides. In some cases, the guide sequence includes at least 2, 3, 4, 5, 6, 7, 8, 9, 10, or more modified nucleotides. In other cases, the guide sequence includes at least 2, 3, 4, 5, 6, 7, 8, 9, 10, 11, 12, 13, 14, 15, 16, 17, 19, 20, or more modified nucleotides. The modified nucleotide can be located at any nucleic acid position of the guide sequence. In other words, the modified nucleotides can be at or near the first and/or last nucleotide of the guide sequence, and/or at any position in between. For example, for a guide sequence that is 20 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, and/or position 20 of the guide sequence. In certain instances, from about 10% to about 30%, e.g., about 10% to about 25%, about 10% to about 20%, about 10% to about 15%, about 15% to about 30%, about 20% to about 30%, or about 25% to about 30% of the guide sequence can comprise modified nucleotides. In other instances, from about 10% to about 30%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the guide sequence can comprise modified nucleotides.

In certain embodiments, the modified nucleotides are located at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and/or at internal positions within the guide sequence.

In some embodiments, the scaffold sequence of the modified gRNA contains one or more modified nucleotides. For example, a scaffold sequence that is about 80 nucleotides in length may have 1 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 76, 77, 78, 79, or 80 modified nucleotides. In some instances, the scaffold sequence includes at least 2, 3, 4, 5, 6, 7, 8, 9, 10, or more modified nucleotides. In other instances, the scaffold sequence includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the scaffold sequence. For example, the modified nucleotides can be at or near the first and/or last nucleotide of the scaffold sequence, and/or at any position in between. For example, for a scaffold sequence that is about 80 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, position 39, position 40, position 41, position 42, position 43, position 44, position 45, position 46, position 47, position 48, position 49, position 50, position 51, position 52, position 53, position 54, position 55, position 56, position 57, position 58, position 59, position 60, position 61, position 62, position 63, position 64, position 65, position 66, position 67, position 68, position 69, position 70, position 71, position 72, position 73, position 74, position 75, position 76, position 77, position 78, position 79, and/or position 80 of the sequence. In some instances, from about 1% to about 10%, e.g., about 1% to about 8%, about 1% to about 5%, about 5% to about 10%, or about 3% to about 7% of the scaffold sequence can comprise modified nucleotides. In other instances, from about 1% to about 10%, e.g., about 1%, about 2%, about 3%, about 4%, about 5%, about 6%, about 7%, about 8%, about 9%, or about 10% of the scaffold sequence can comprise modified nucleotides.

In certain embodiments, the modified nucleotides are located at the 3′-end (e.g., the terminal nucleotide at the 3′-end) or near the 3′-end (e.g., within 1, 2, 3, 4, or 5 nucleotides of the 3′-end) of the scaffold sequence and/or at internal positions within the scaffold sequence.

In some embodiments, the modified gRNA comprises one, two, or three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one, two, or three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In some instances, the modified gRNA comprises one modified nucleotide at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one modified nucleotide at the 3′-end (e.g., the terminal nucleotide at the 3′-end) or near the 3′-end (e.g., within 1, 2, 3, 4, or 5 nucleotides of the 3′-end) of the scaffold sequence.

In other instances, the modified gRNA comprises two consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and two consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In yet other instances, the modified gRNA comprises three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In particular embodiments, the modified gRNA comprises three consecutive modified nucleotides at the 5′-end of the guide sequence and three consecutive modified nucleotides at the 3′-end of the scaffold sequence.

The modified nucleotides of the gRNA can include a modification in the ribose (e.g., sugar) group, phosphate group, nucleobase, or any combination thereof. In some embodiments, the modification in the ribose group comprises a modification at the 2′ position of the ribose.

In some embodiments, the modified nucleotide includes a 2′fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), a phosphodiamidate morpholino, or a combination thereof.

Modified nucleotides or nucleotide analogues can include sugar- and/or backbone-modified ribonucleotides (i.e., include modifications to the phosphate-sugar backbone). For example, the phosphodiester linkages of a native or natural RNA may be modified to include at least one of a nitrogen or sulfur heteroatom. In some backbone-modified ribonucleotides, the phosphoester group connecting to adjacent ribonucleotides may be replaced by a modified group, e.g., a phosphothioate group. In preferred sugar-modified ribonucleotides, the 2′ moiety is a group selected from H, OR, R, halo, SH, SR, NH₂, NHR, NR₂ or ON, wherein R is C₁-C₆ alkyl, alkenyl or alkynyl and halo is F, Cl, Br or I. In some embodiments, the sugar-modified ribonucleotide comprises a 2′-O-methyl nucleotide.

In some embodiments, the modified nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the modified gRNA contains one or more 2′-fluoro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g., from U.S. Pat. Nos. 4,373,071, 4,458,066, 4,500,707, 4,668,777, 4,973,679, 5,047,524, 5,132,418, 5,153,319, 5,262,530, and 5,700,642. Numerous modified nucleosides and modified nucleotides that are suitable for use as described herein are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the modified gRNA described herein includes a nucleobase-modified ribonucleotide, i.e., a ribonucleotide containing at least one non-naturally occurring nucleobase instead of a naturally occurring nucleobase. Non-limiting examples of modified nucleobases which can be incorporated into modified nucleosides and modified nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-O-ribosyladenosine(phosphate)), I (inosine), m11 (1-methylinosine), m′Im (1,2′-O-dimethylinosine), m3C (3-methylcytidine), Cm (2T-O-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5 s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), m62Am (N6,N6,O-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-0-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxy cytosine, 5-(C₁-C₆)-alkylcytosine, 5-methylcytosine, 5 (C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

In some embodiments, the phosphate backbone of the modified gRNA is altered. The modified gRNA can include one or more phosphorothioate, phosphoramidate (e.g., N3′-P5′-phosphoramidate (NP)), 2′-O-methoxy-ethyl (2′MOE), 2′-O-methyl-ethyl (2′ME), and/or methylphosphonate linkages. In certain instances, the phosphate group is changed to a phosphothioate, 2′-O-methoxy-ethyl (2′MOE), 2′-O-methyl-ethyl (2′ME), N3′-P5′ phosphoramidate (NP), and the like. In some embodiments, the modified gRNA includes one or more 2′-O-methyl, 3′-phosphorothioate and/or 2′-O-methyl, 3′thioPACE nucleotides.

It should be noted that any of the modifications described herein may be combined and incorporated in the guide sequence and/or the scaffold sequence of the modified gRNA.

In some cases, the modified gRNAs also include a structural modification such as a stem loop, e.g., M2 stem loop or tetraloop.

The chemically modified gRNAs can be used with any CRISPR-associated or RNA-guided technology. As described herein, the modified gRNAs can serve as a guide for any Cas9 polypeptide or variant thereof, including any engineered or man-made Cas9 polypeptide. The modified gRNAs can target DNA and/or RNA molecules in isolated cells or in vivo (e.g., in an animal).

### V. Compositions and Methods of the Invention

The ability to program CRISPR-Cas nucleases (e.g., Cpf1 (also known as Cas12a) and Cas9) to nick has uses in technology and implications in the CRISPR immune biology. The ability to tailor some CRISPR-Cas nucleases to nick at specific sites “on demand” provides a potential alternative to mutated CRISPR nuclease variants. While mutation of defined cleavage domains^{8,21,22} provides a capability for nicking activities for some CRISPR nucleases, the approach is not available for all Cas systems (particularly Cpf1, where a single domain may execute both cleavage reactions²³⁻²⁵). In addition, such approaches limit the multifunctional applications of CRISPR in a single system, since co-expression of nickase and wild-type enzymes will generate cleavage at intended nickase sites as well as nicking at intended cleavage sites. As described herein, use of one or more wild-type enzymes with gRNAs for nicking and cleavage would surmount this challenge, with tuning of gRNAs a likely requirement in making such an approach effective. The use of mismatched gRNAs with wild-type CRISPR enzyme to direct nicking could be valuable for gene editing or replace nickases in gene manipulation. As an example, base editors composed of a catalytically dead Cas9 fused to a deaminase could potentially be redesigned to involve a fully functional Cas9 fused to a deaminase that can be guided to specific areas via mismatched gRNAs.

A fraction of Cas9's observed activities in vivo either in native systems or in engineering applications may reflect nicking rather than cleavage activities, with the balance likely dependent on in vivo conditions as well as the sequences of gRNAs and targets. Of particular importance here, observations that nicking activities can provide advantages in genome editing (e.g., Davis et al.¹², Gao et al.³⁷, and Satomura et al.³⁸) indicate that such conditions could prove advantageous. The consequences of nicking may depend in each system on the kinetic balances between nick ligation, single stranded exo- and endonuclease activities that might extend or convert nicks³⁹, other modes of DNA repair, and DNA replication/division rates. These kinetic parameters may vary substantially based on intrinsic cellular properties, on specific genomic positions, and on stochastic ordering of events.

An understanding of sequence requirements for nicking as well as full cleavage of targets is critical for identification and assessment of potential off-target effects of CRISPR-Cas nucleases as these are applied in experimental, biotechnological, and clinical settings. In particular, the existence of a guide-specific nicking repertoire impacts the selection of gRNA targets to avoid off-target consequences during genome editing. Although nicked double stranded DNA in vivo is less detrimental than a double stranded break, nicks lead to downstream repair events that can cause unexpected mutations (e.g., Kuzminov et al.²⁶). Of the various algorithms that score and/or pick gRNAs based on potential off-target effects^{27,28}, many entail a user-selected threshold for candidate gRNA mismatch and/or leave the user to select candidate gRNAs based a mismatch-count-based estimate of off-target cleavage potential. Understanding the nicking abilities of CRISPR-Cas nucleases may thus offer considerable value in gRNA design and selection.

Imperfect homology-dependent nicking of CRISPR-Cas nucleases has implications on the mechanism of spacer acquisition in CRISPR immunity. Spacer acquisition is the process in which nucleic acids from foreign genetic elements (e.g., plasmids, bacteriophages, etc.) are integrated in the CRISPR loci. The integrated spacers are later transcribed and processed and used for host defense^{1,29}. Cas1 and Cas2 catalyze spacer acquisition^{30,31}. It has been shown that the effector CRISPR-Cas nucleases (nuclease active and inactive) in type II CRISPR systems are necessary for spacer acquisition^{32,33}. In addition, there is evidence that type I CRISPR-Cas systems in the presence of non-targeting gRNAs can increase spacer acquisition with evident strand bias, a phenomenon that is called “primed adaptation”³⁴⁻³⁶. Although primed adaptation has not been reported for the type II systems to this date, it is possible that spacer acquisition is conserved among CRISPR systems. These observations in conjunction with knowledge that nicking can be induced with mismatch gRNAs could be relevant in the increased ability to acquire spacers in the presence of a non-targeting gRNAs. In particular, the nicked product could provide a strand specific advantage for the integration of nucleic acids in the CRISPR loci. An additional possibility is that the minimal homology between the gRNA and DNA target produces a nick that serves as an anchor for the CRISPR-Cas nuclease to recruit Cas1 and Cas2.

The present invention features compositions and methods that include one or more CRISPR-Cas nucleases (e.g., Cpf1 (also known as Cas12a) and Cas9; a wild-type or mutant CRISPR-Cas nuclease) and one or more gRNAs having at least one nucleotide mismatch or deletion relative to a target DNA sequence. A dual nickase CRISPR system of the disclosure may comprise (a) a first CRISPR-Cas nickase comprising a first CRISPR-Cas nuclease (e.g., a wild-type or mutant CRISPR-Cas nuclease) and a first guide RNA (gRNA) comprising a first targeting region capable of guiding the first nuclease to cleave one strand of a double-stranded target DNA sequence, wherein the first gRNA comprises at least one nucleotide mismatch or deletion in the first targeting region relative to the target DNA sequence; and (b) a second CRISPR-Cas nickase comprising a second CRISPR-Cas nuclease (e.g., a wild-type or mutant CRISPR-Cas nuclease) and a second gRNA comprising a second targeting region capable of guiding the second nuclease to cleave the complementary strand of the target DNA sequence, wherein the second gRNA comprises at least one nucleotide mismatch or deletion in the second targeting region relative to the target DNA sequence. The disclosure also features a composition comprising a first RNA-guided DNA nuclease (e.g., a wild-type or mutant RNA-guided DNA nuclease), a first guide RNA (gRNA), a second RNA-guided DNA nuclease (e.g., a wild-type or mutant RNA-guided DNA nuclease), and a second gRNA, wherein the first gRNA comprises at least one nucleotide mismatch or deletion relative to a region of a first strand of a target DNA, wherein the second gRNA comprises at least one nucleotide mismatch or deletion relative to a region of a second strand of the target DNA, wherein the first gRNA guides the first nuclease to bind to and cleave in the region of the first strand of the target DNA, wherein the second gRNA guides the second nuclease to bind to and cleave in the region of the second strand of the target DNA, and wherein cleavage of the first and second strands of the target DNA by the first and second nucleases produces a double-strand break in the target DNA.

The invention also features methods of cleaving both strands of a target DNA by providing the dual nickase CRISPR system described above. In some embodiments, the methods of cleaving both strands of a target DNA may comprise providing a RNA-guided DNA nickase (e.g., a wild-type or mutant RNA-guided DNA nuclease), a first gRNA, and a second gRNA, in which the first gRNA comprises at least one nucleotide mismatch to a region of a first strand in the target DNA, the second gRNA comprises at least one nucleotide mismatch to a region of a second strand in the target DNA, the first gRNA guides the RNA-guided DNA nickase to bind to and cleave in the region of the first strand of the target DNA, the second gRNA guides the RNA-guided DNA nickase to bind to and cleave in the region of the second strand of the target DNA, and cleavage of both strands by the RNA-guided DNA nickase produces two product DNAs each having an overhang of at least one nucleotide.

The invention also features compositions and methods of nicking one strand of a target DNA by providing a nickase CRISPR system comprising: a CRISPR-Cas nickase comprising a CRISPR-Cas nuclease (e.g., a wild-type or mutant CRISPR-Cas nuclease) and a gRNA comprising a targeting region capable of guiding the nuclease to cleave one strand of a double-stranded target DNA sequence, wherein the gRNA comprises at least one nucleotide mismatch or deletion in the targeting region relative to the target DNA sequence. A composition that nicks one strand of a target DNA may comprise a RNA-guided DNA nuclease (e.g., a wild-type or mutant RNA-guided DNA nuclease) and a gRNA, wherein the gRNA comprises at least one nucleotide mismatch or deletion relative to a region in a target DNA, wherein the gRNA guides the nuclease to bind to and cleave in the region in the target DNA, and wherein cleavage of the target DNA by the nuclease produces a single-strand break in the target DNA. In some embodiments of the composition that nicks one strand of a target DNA, the at least one nucleotide mismatch or deletion relative to the region in the target DNA is in a distal region of the gRNA. In some embodiments, the at least one nucleotide mismatch relative to the region in the target DNA is a transversion point mutation. In other embodiments of the composition, the gRNA comprises a nucleotide deletion in a seed region and a nucleotide mismatch in a distal region of the of the gRNA relative to the region in the target DNA. In yet some embodiments, the gRNA comprises a nucleotide mismatch that is a transversion point mutation in a seed region and a nucleotide mismatch that is a transversion point mutation in a distal region of the of the gRNA relative to the region in the target DNA.

In some embodiments of the compositions and methods that nick one strand of a target DNA, the nicked site in the target DNA may serve as a labeling site and be further modified. In some embodiments, additional nucleotides may be inserted at the nicked site. The additional nucleotides may function as a site of recognition and binding for other proteins. The additional nucleotides may also serve targeting purposes, for example, directing the target DNA to a specific location inside a cell. In further embodiments, once the RNA-guided DNA nuclease is directed to the target DNA by the gRNA and nicks one strand of the target DNA, the RNA-guided DNA nuclease may be immobilized on the target DNA.

In some embodiments of the compositions and methods described herein, the at least one nucleotide mismatch or deletion in the first gRNA relative to the region of the first strand of the target DNA is in a distal region of the first gRNA. In some embodiments, the at least one nucleotide mismatch or deletion in the second gRNA relative to the region of the second strand of the target DNA is in a distal region of the second gRNA. Moreover, in some embodiments, the at least one nucleotide mismatch in the first gRNA relative to the region of the first strand of the target DNA is a transversion point mutation. In some embodiments, the at least one nucleotide mismatch in the second gRNA relative to the region of the second strand of the target DNA is a transversion point mutation.

In particular embodiments of the compositions and methods described herein, the first gRNA comprises a nucleotide deletion in a seed region and a nucleotide mismatch in a distal region of the of the first gRNA relative to the region of the first strand of the target DNA. In some embodiments, the second gRNA comprises a nucleotide deletion in a seed region and a nucleotide mismatch in a distal region of the second gRNA relative to the region of the second strand of the target DNA.

In other particular embodiments of the compositions and methods described herein, the first gRNA comprises a nucleotide mismatch that is a transversion point mutation in a seed region and a nucleotide mismatch that is a transversion point mutation in a distal region of the of the first gRNA relative to the region of the first strand of the target DNA. In some embodiments, the second gRNA comprises two nucleotide mismatches that are transversion point mutations in a distal region of the second gRNA relative to the region of the second strand of the target DNA.

### VI. Introducing DNA Nucleases and gRNAs into Cells

The compositions and methods of the present invention including one or more CRISPR-Cas nucleases (e.g., Cpf1 (also known as Cas12a) and Cas9) and one or more gRNAs having at least one nucleotide mismatch or deletion relative to a target DNA sequence may be used to nick or cleave the target DNA sequence inside a cell without modification of the nuclease protein, thus, utilizing the gRNAs with a variety of patterns of mismatch to the target DNA sequence. Methods for introducing polypeptides and nucleic acids into a cell are known in the art. Any known method can be used to introduce a polypeptide or a nucleic acid (e.g., a nucleotide sequence encoding the DNA nuclease or a gRNA) into a cell, e.g., a mammalian cell (e.g., a human cell). Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments, the polypeptide and/or nucleic acids of the compositions and methods of the invention can be introduced into a cell using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct) and one or more nucleic acid components such as a gRNA and/or a donor template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

### VII. Methods for Isolating and Purifying Genetically Modified Cells

Selectable markers, detectable markers, cell surface markers, and cell purification markers, alone or in combination, can be used to isolate and/or purify genetically modified cells. Expression of a selectable marker gene encoding an antibiotic resistance factor can provide for preferential survival of genetically modified cells in the presence of the corresponding antibiotic, whereas other cells present in the culture will be selectively killed. Alternatively, expression of a fluorescent protein such as GFP or expression of a cell surface marker not normally expressed on the cells may permit genetically modified cells to be identified, purified, or isolated by fluorescence-activated cell sorting (FACS), magnetic-activated cell sorting (MACS), or analogous methods. Examples of cell surface markers include CD8, truncated CD8, CD19, truncated CD19, truncated nerve growth factor receptor (tNGFR), and truncated epidermal growth factor receptor (tEGFR), although other cell surface markers can also fulfill the same function.

Methods for isolating or purifying the genetically modified cells are known in the art. In some embodiments, a population of genetically modified cells is isolated or purified (e.g., separated) from a population of unmodified cells in accordance with the enrichment scheme of the present invention. For example, FACS or MACS methods can be used to enrich genetically modified human cells expressing a fluorescent protein such as GFP or a cell surface marker such as truncated nerve growth factor receptor (tNGFR) as described herein.

Methods for culturing or expanding the genetically modified cells are known in the art. Methods for culturing cells and their progeny are known, and suitable culture media, supplements, growth factors, and the like are both known and commercially available. In some embodiments, human cells are maintained and expanded in serum-supplemented or serum-free conditions. In some embodiments, the isolated or purified gene modified cells can be expanded in vitro according to standard methods known to those of ordinary skill in the art.

### VIII. Examples

The following examples are offered to illustrate, but not to limit, the claimed invention.

### Example 1. Methods

Variant Plasmid Library

A mixed variant library for Cpf1 was cloned following Fu et al.¹⁹. A Cas9 unc-22A variant library created using degenerate oligonucleotide synthesis and a library created using pooled oligonucleotide synthesis were previously characterized in Fu et al.²⁰ and were used in this study. The library created by pooled oligonucleotide synthesis was retransformed and re-grown from Fu et al.²⁰.

High Throughput In Vitro Target Specificity Assays

Cpf1 in vitro target specificity assays were performed as reported with Fu et al.¹⁹, in 100 mM NaCl, 50 mM Tris-HCl, 10 mM MgCl₂, 100 μg/ml BSA, pH 7.9. Cpf1 gRNAs were synthesized by Integrated DNA Technologies.

For Cas9, the modified single (chimeric) gRNA structure of Jinek et al.⁸ was used. Target specificity in vitro assays for Cas9 were performed as detailed in Fu et al.¹⁹ (reaction buffer: 20 mM Tris-HCl, 10 mM (NH₄)₂SO₄, 10 mM KCl, 2 mM MgSO₄, 0.1% Triton X-100, pH 8.8). In addition to the buffer used for these studies, a set of parallel assays were performed using an alternative buffer similar to that of Jinek et al.⁸ (reaction buffer: 20 mM HEPES, 100 mM NaCl, 5 mM MgCl₂, 0.1 mM EDTA, pH 6.5). Similar results were obtained in the two buffers, albeit (as previously observed; Fu et al.²⁰) with differences in cleavage kinetics and completion.

Calculation of Retention Scores

For each sequence ‘X’:

Retention[X]=log₂(Representation_{X}[Library with addition of CRISPR-Cas Nuclease]/Representation_{X}[(Uncleaved Library) or (All Variants of a specified target)]

First to total reads from each experimental condition that have the expected length (35-36 bps) and barcode (scaling to total library counts) were normalized. As an alternative normalization for comparison, different libraries were scaled by counting tags matching a non-targeted gRNA sequence or all non-targeted gRNA sequences. For experiments with gRNAs targeting EGFP-1, EGFP-2, and unc-22A, the library was normalized to the sum of all variants of the rol-6 target sequence or normalized to the sum of all non-targeted variants. For experiments where the rol-6 was the intended target, the library was normalized to a total sum of unc-22A variants. As expected, the different normalization approaches yield highly comparable results. Normalizations used for display are noted for each figure.

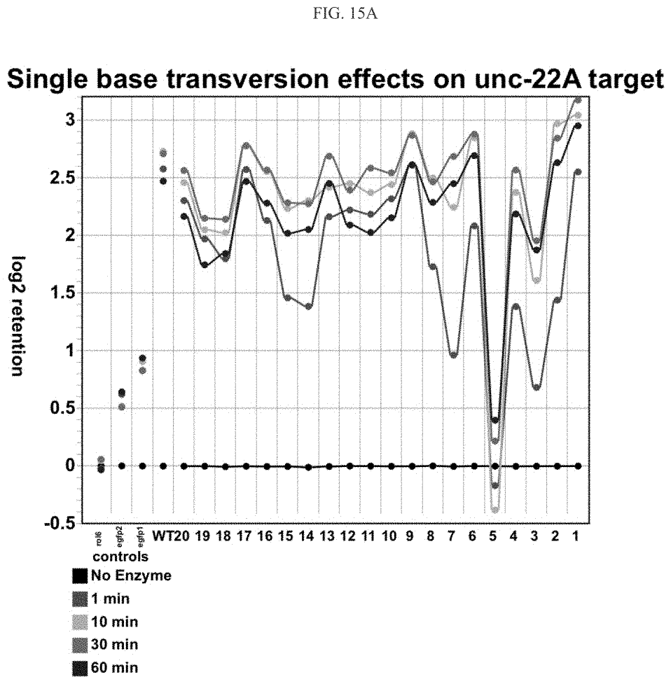

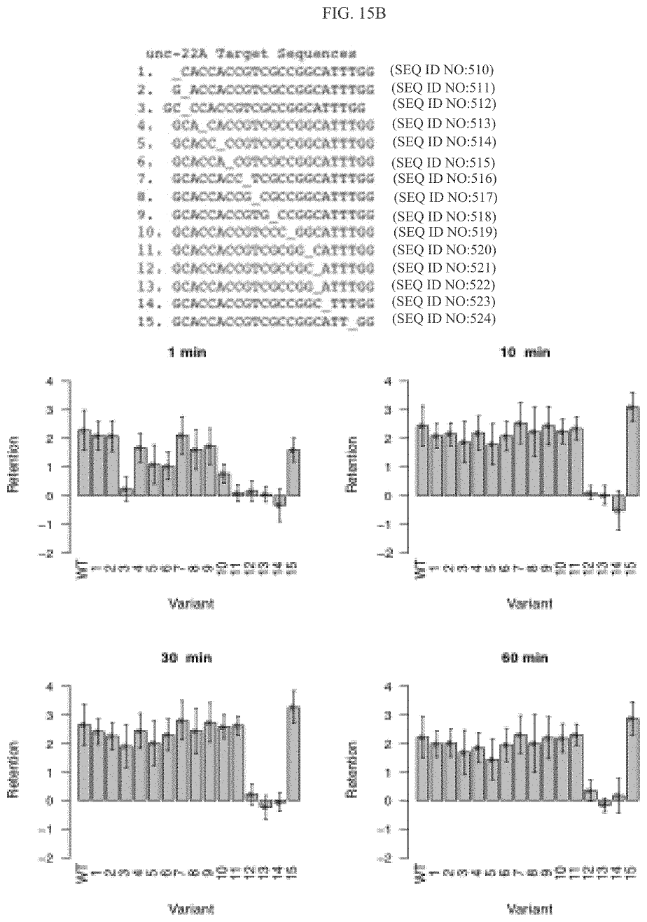

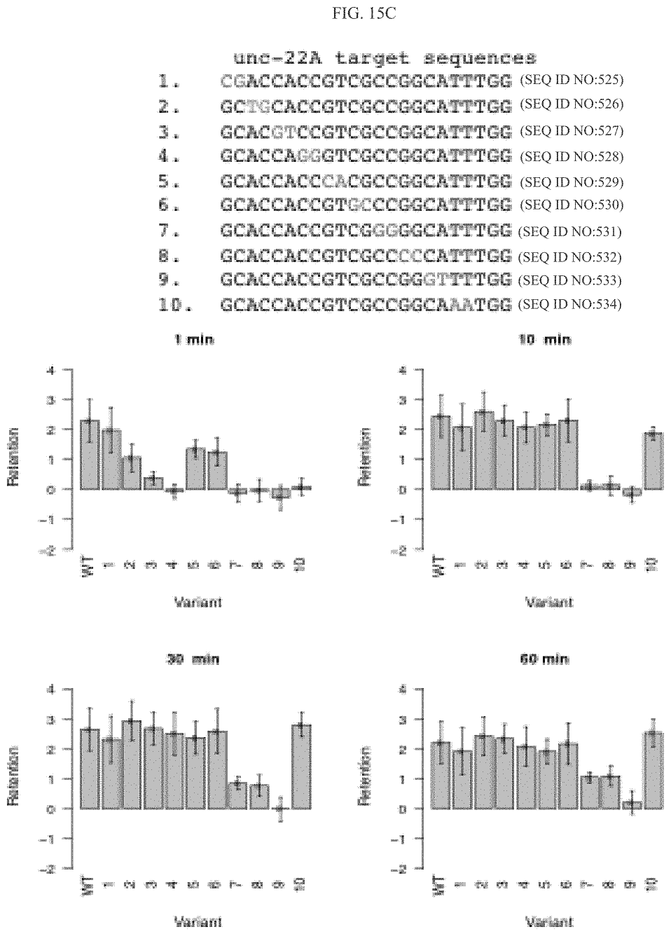

Cases of positive retention (as noted above and in [reference to increased yield for nicked circles in PCR]) are indicative of potential target nicking rather than cleavage. A maximum positive retention in the experiments was approximately 3 log-2 units, yielding a comparable estimate. A set of assays using Cas9 D10A nickase also allows us a more quantitative conversion between degree of enhancement in PCR yield and quantitative fraction of nicked templates (100% nicking yields a retention ˜2.5-3, again in the same range; FIGS. 15A-15C).

Nicking Agarose Gel Assays

Target DNA were cloned into a parent vector (pHRL-TK, Promega) using Notl and Acc65I restriction sites. Cpf1 or Cas9 reactions were set up as above for the library assays. For time points, samples were stopped with 100 mM EDTA, 2% SDS, and 80 U/μL Proteinase K and/or flash frozen with dry ice and separated on ˜1-1.5% agarose gel (TAE buffer, Ethidium Concentration [0.3 mg/L]).

Protein Components

As/Fn/Lb-Cpf1 constructs were generated by assembly of synthetic gene fragments into an E. coli expression plasmid using the NEBuilder HiFi DNA Assembly Cloning Kit (NEB #E5520S). As/Fn/Lb-Cpf1 expression vectors contained N-terminal 6×His-tag and SV40 NLS, and C-terminal SV40 NLS. Recombinant proteins were expressed in modified E. coli NiCo21 (DE3) cells (NEB C2925H) harboring the Cpf1 expression plasmid by growing in LB at 23° C. for 16 hr in presence of IPTG at 0.4 mM. Cells were disrupted by sonication prior to chromatographic purification.

As/Fn/Lb-Cpf1 was purified using HiTrap DEAE^{FF} (GE Healthcare), HisTrap^{HP} (Ni-NTA) (GE Healthcare) and HiTrapSP^{HIP} (GE Healthcare) columns. Recombinant proteins were dialyzed and concentrated into 20 mM Tris-HCl (pH7.4), 500 mM NaCl, 1 mM DTT, 0.1 mM EDTA and 50% glycerol.

Cas9 and LbCpf1 protein preparations were NEB M0386S and M0653S respectively.

Cas9 In Vivo Assays

Two yeast strains were transfected with a pooled library cloned into a plasmid carrying a Cas9 expression cassette (driven by the GalL promoter), a gRNA expression construct (driven by a tetracyline inducible RPR1 promoter), and a target or non-target sequence adjacent to a potential “PAM” site (‘NGG’) (plasmid map available here: benchling.com/s/O5VobNjd). Target sequences were inserted into the plasmid at a defined location, with the majority deriving from a library of variants of the unc-22A, while variants of a sequence of unrelated origin (PS4) provided a number of internal controls¹⁹. The two yeast yeast populations analyzed were from strains BY4741 (BY) and KU70 deletion strain (KU) from MATa collection. BY was an auxotrophic wild-type S288C lab strain, while KU was chosen for analysis based on its intrinsic defects in non-homologous double strand break repair. No major difference was found between the two strains. Each strain was analyzed without specific induction of Cas9 (“baseline levels”). Additionaly, each strain was analyzed after one division and at 2.5 generations as assessed by optical density (these were ˜90 and ˜230 minutes after an initial 4 hour metabolic adaptation to galactose media from dextrose) following induction with galactose and 250 ng/ml anhydrotetracycline (ATc). Control, single-generation, and 2.5 generation time points in yeast were analyzed in duplicate for KU70, with control and single generation time points analyzed in duplicate for BY4741.

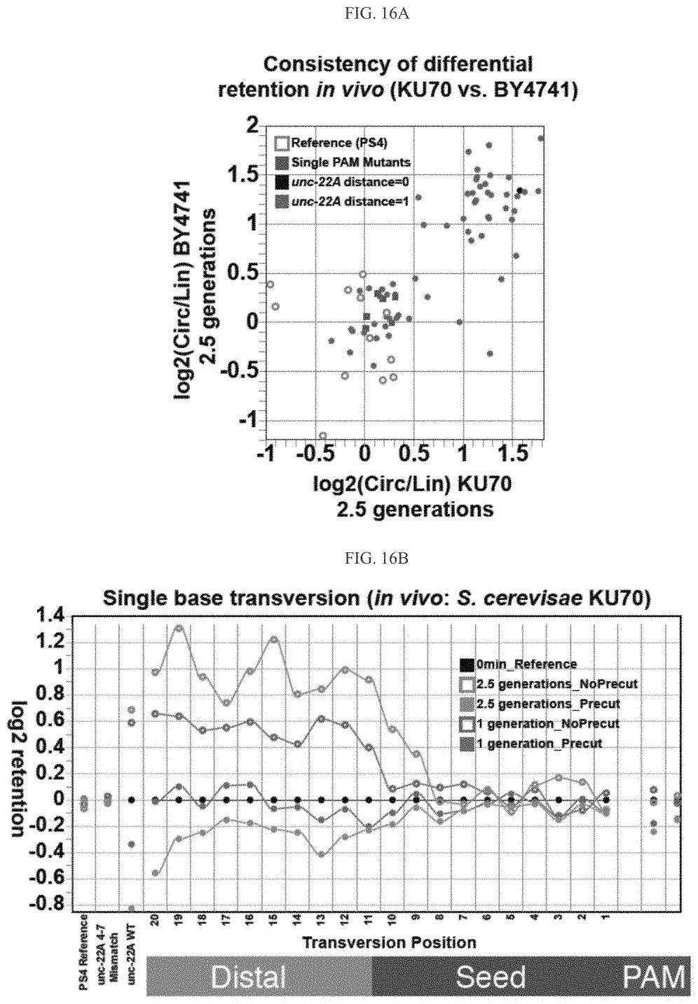

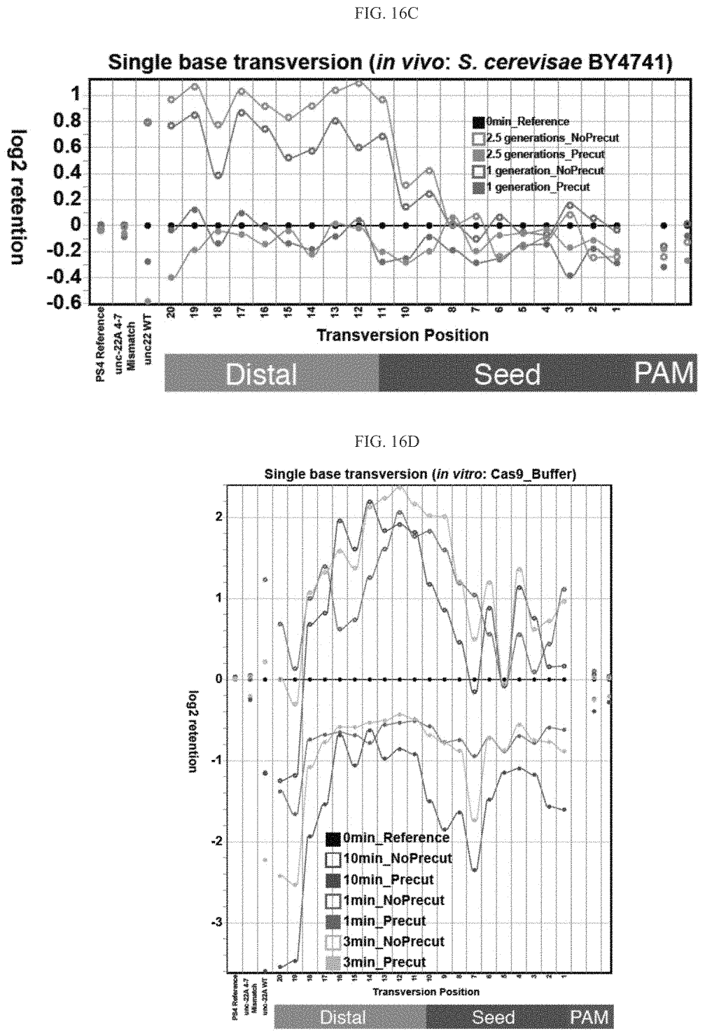

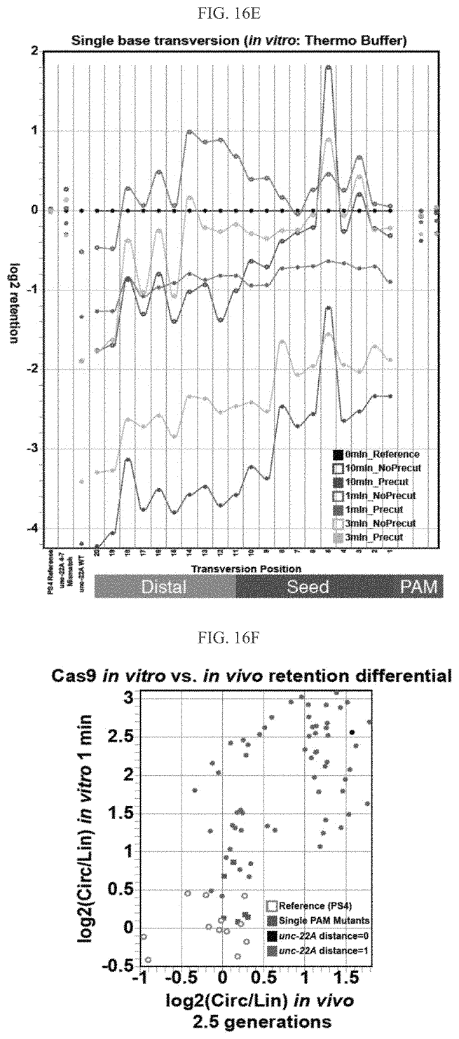

In the analysis (FIGS. 16A-16F), the difference in log retention score was used for any given target (circular-linear), as the provisional metric for evaluation, validating the measurement as described. Retention scores for these comparisons were calculated from individual DNA pools that had been subdivided following extraction. One aliquot was analyzed following an experimental template-linearization cleavage step (designated as “linear” assay a.k.a. Method 1), while a second aliquot was analyzed without such a step (designated as “circular” assay a.k.a. Method 2). As noted above, previous biochemical analysis¹⁸ and the inventors' own analysis in vitro confirmed that the assessment described herein provided a metric that assessed nicking of the DNA.

### Example 2. Cpf1 Nicking and Cleavage Specificity

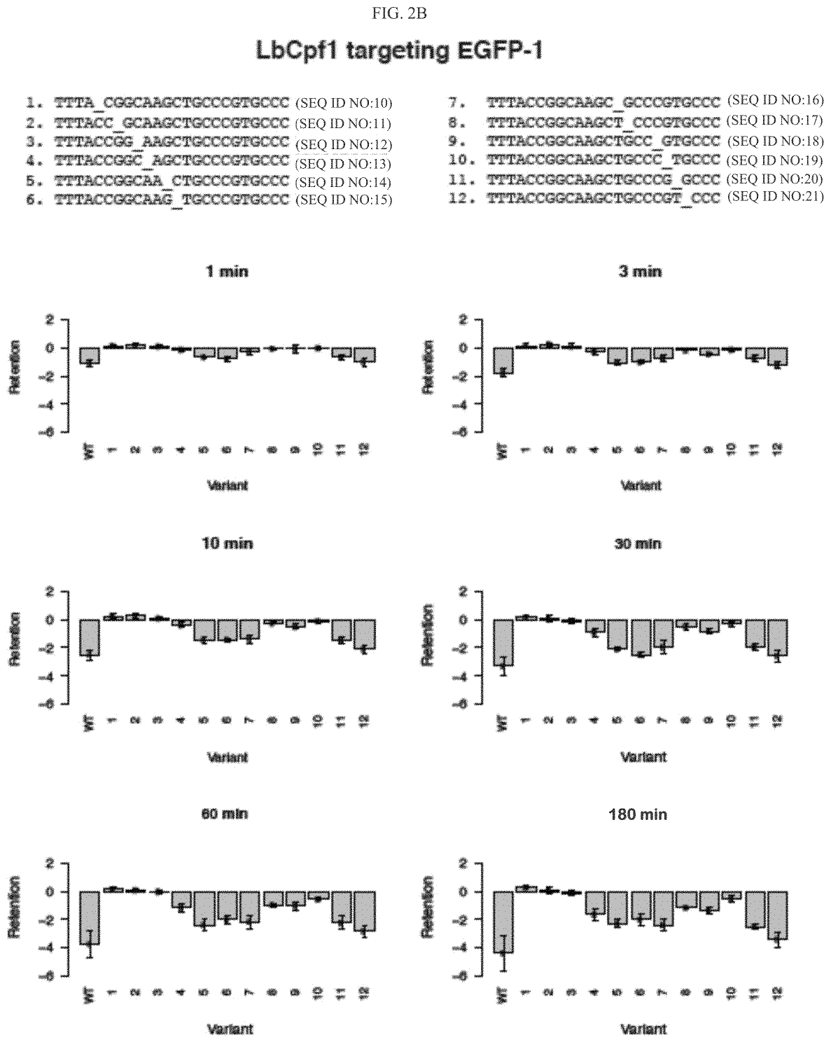

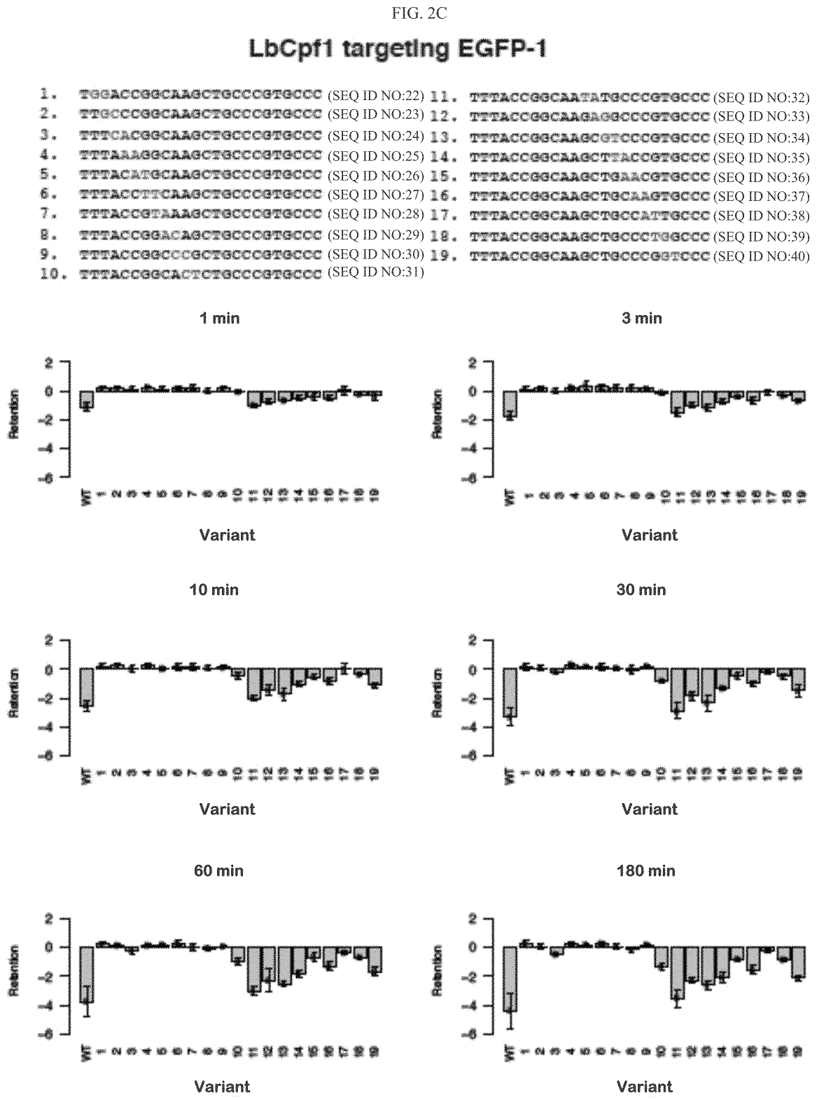

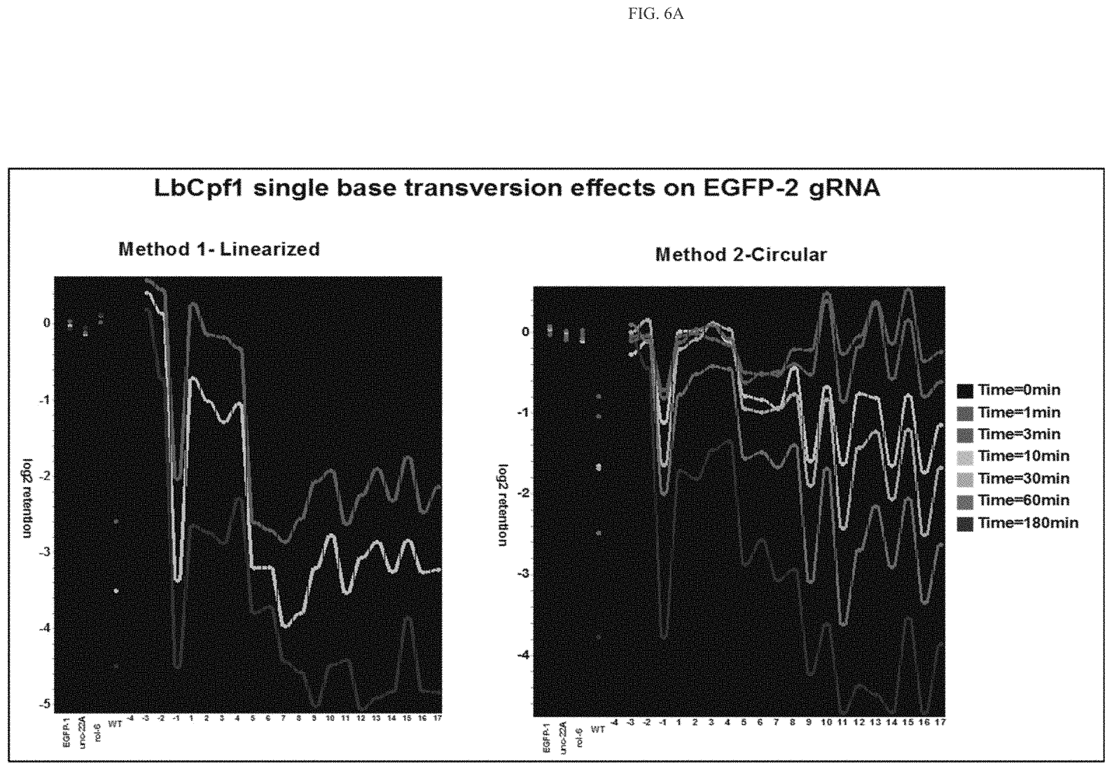

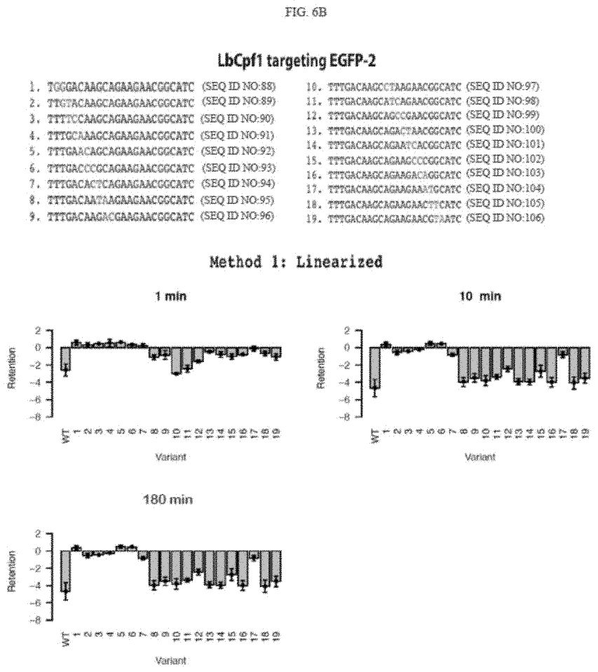

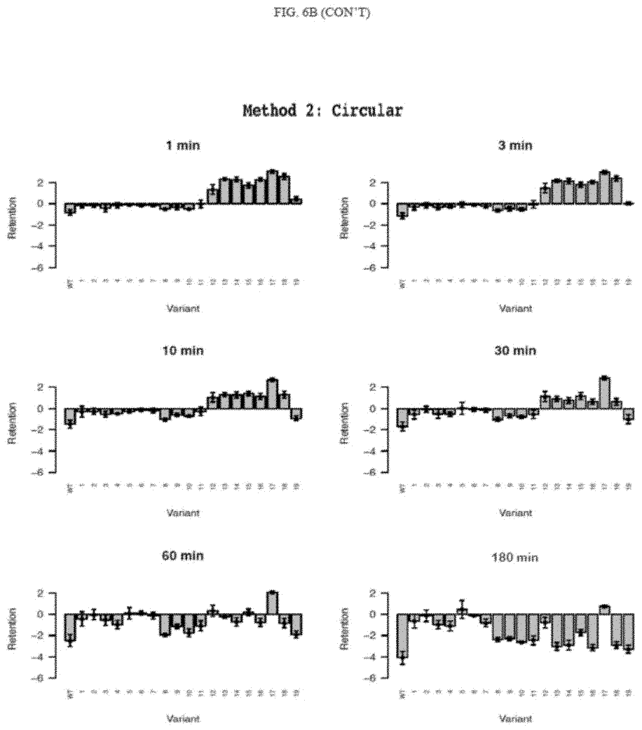

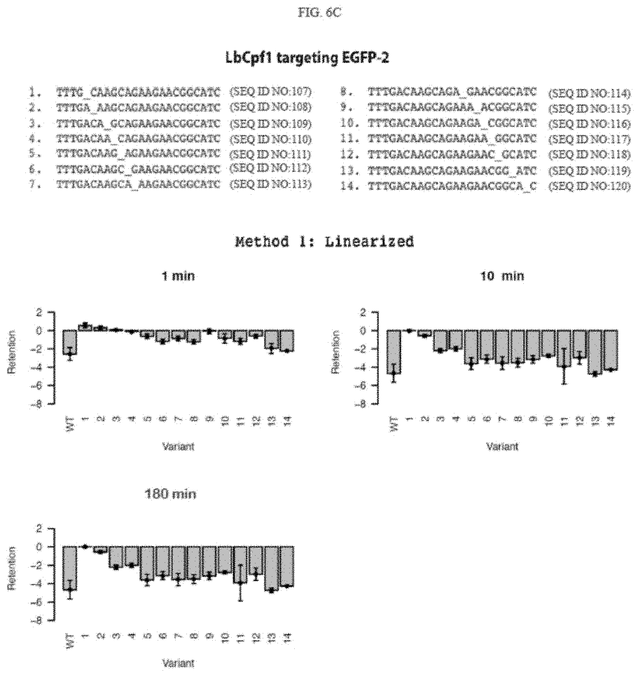

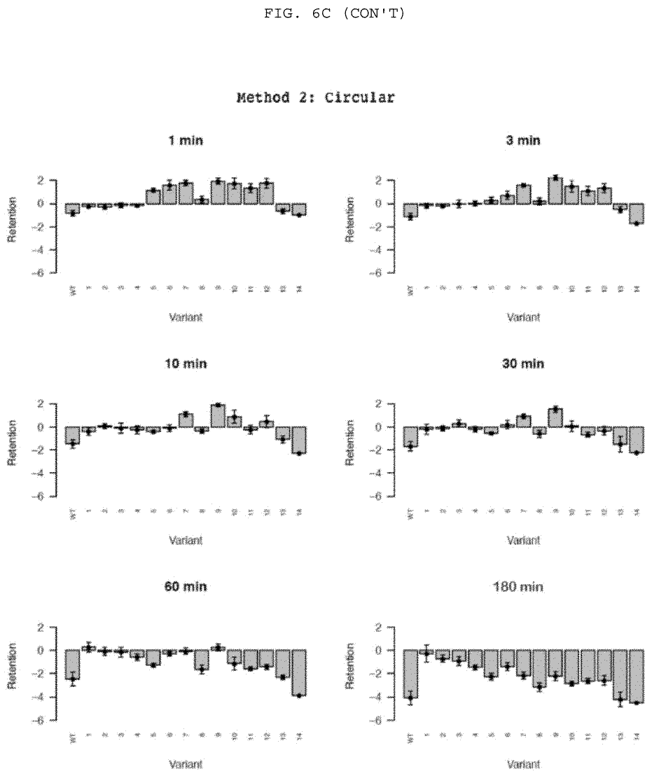

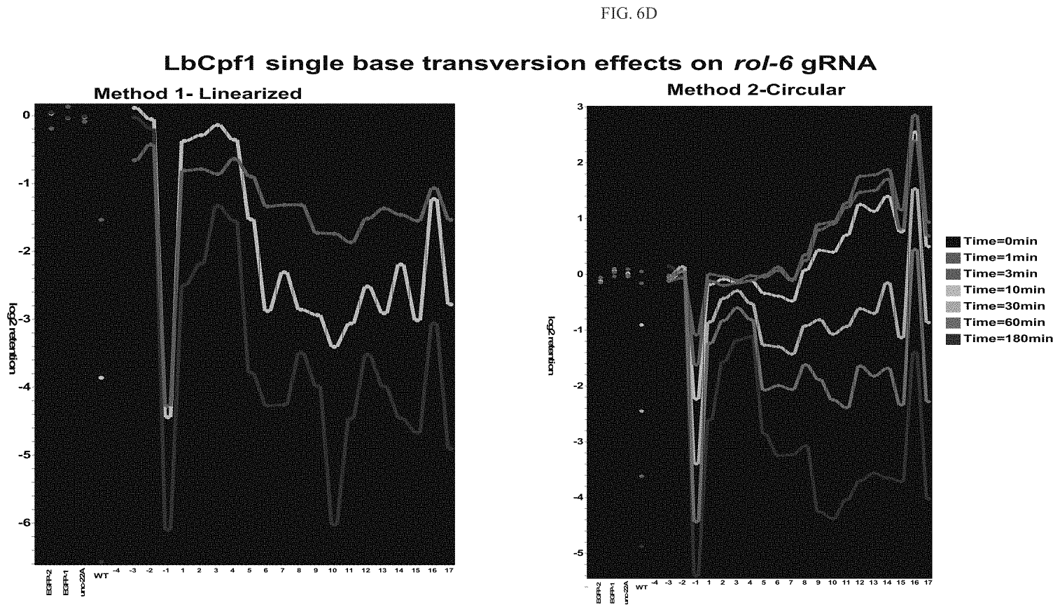

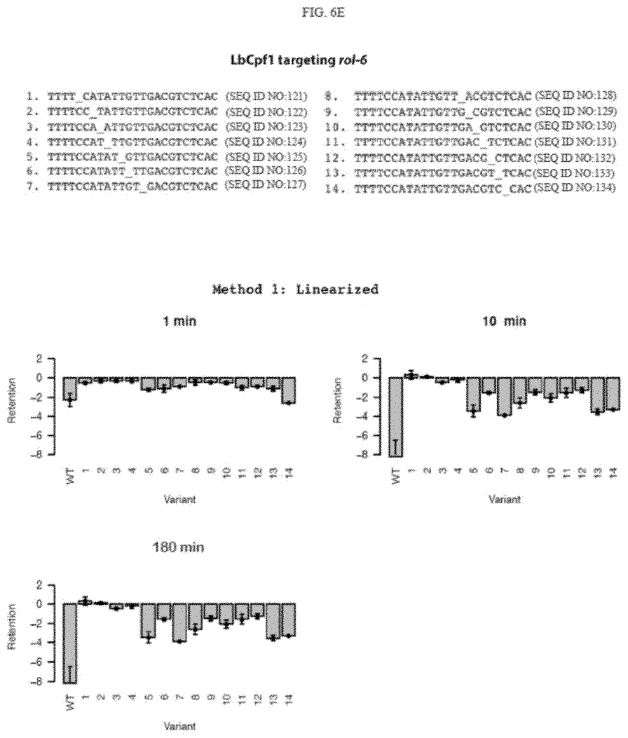

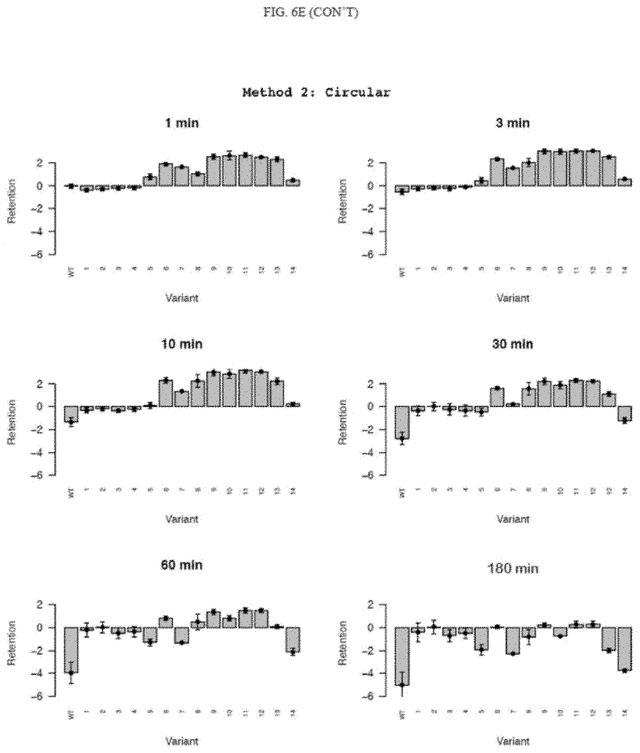

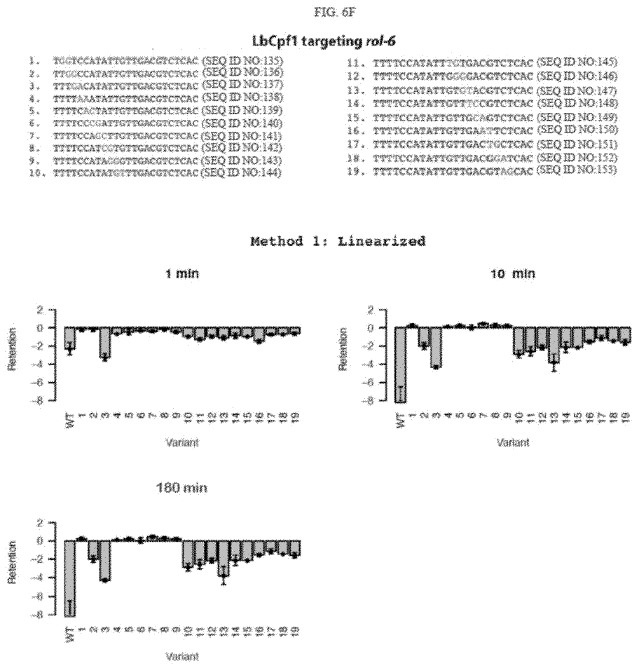

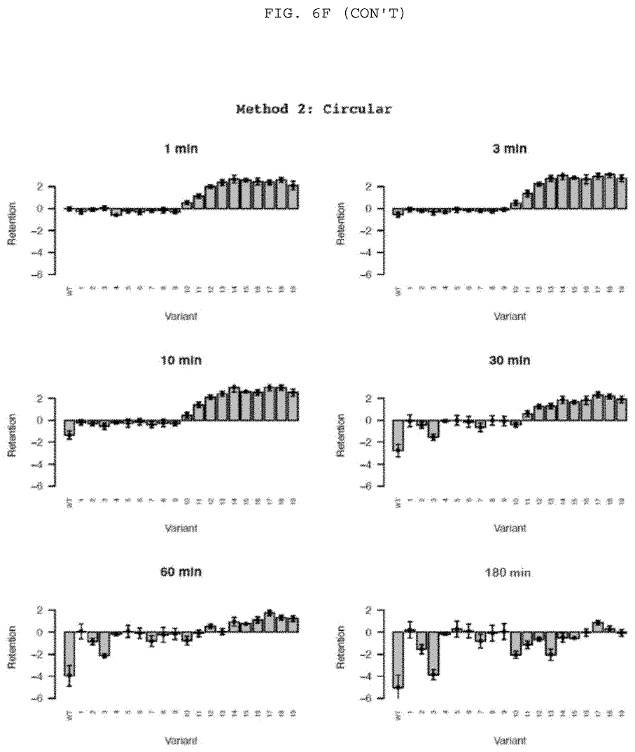

Sequence dependence for LbCpf1 activity was investigated using a variant library of plasmids for four different canonical guide sequences: EGFP-1, EGFP-2, unc-22A, and rol-6 (FIG. 1B). Initial assays were carried out with the backbone-linearization step (Method 1, FIG. 1A). For each target, the libraries contained unmodified sequences, single-base variants, double-adjacent transversions, and deletions. Using loss-of-amplification across the cleavage junction as an assay, LbCpf1 showed a similar specificity profile to previous characterizations of Cas9, less tolerance of mutations in the seed region (positions 1-10, with position 1 being PAM-proximal) while sequence requirements in the distal region (positions 11-20) were more lax (FIGS. 2A-2C; FIGS. 6A-6F). This trend is consistent for all three classes of variants, for all Cpf1s, and on all tested targets (FIGS. 6A-6F, 7A-7L, and 8A-8I).

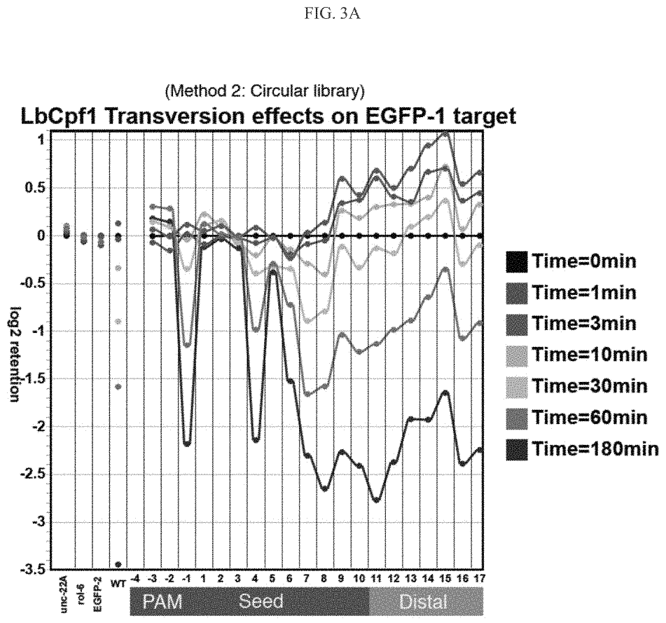

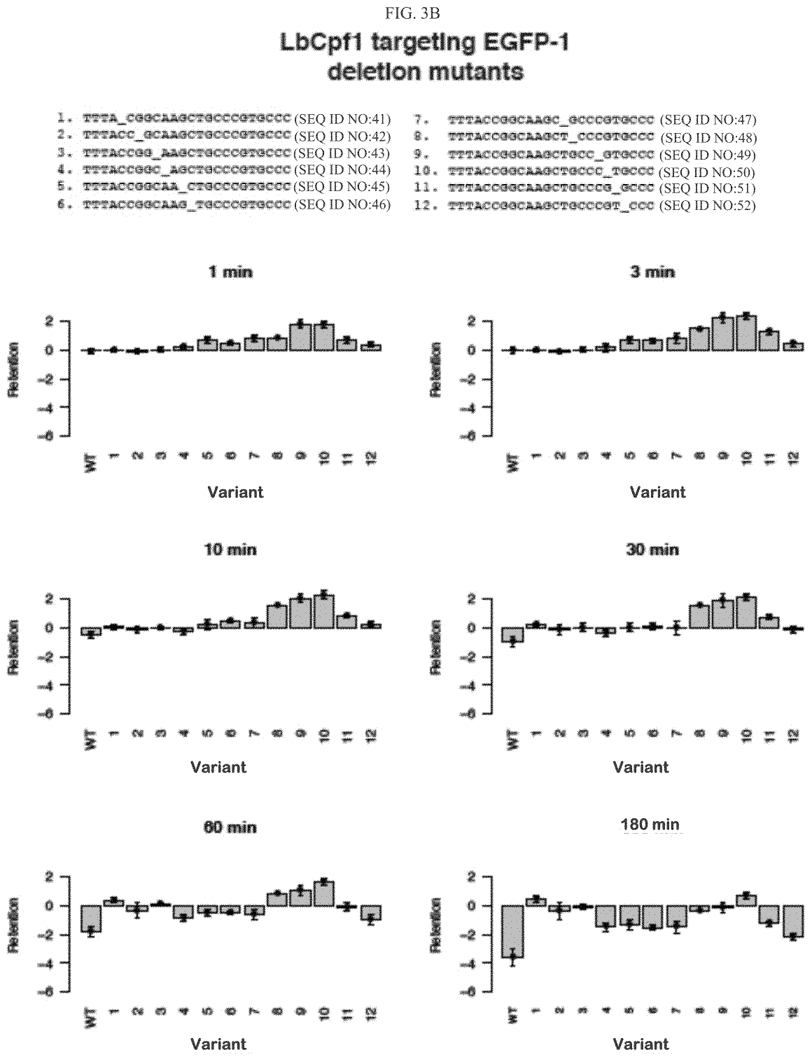

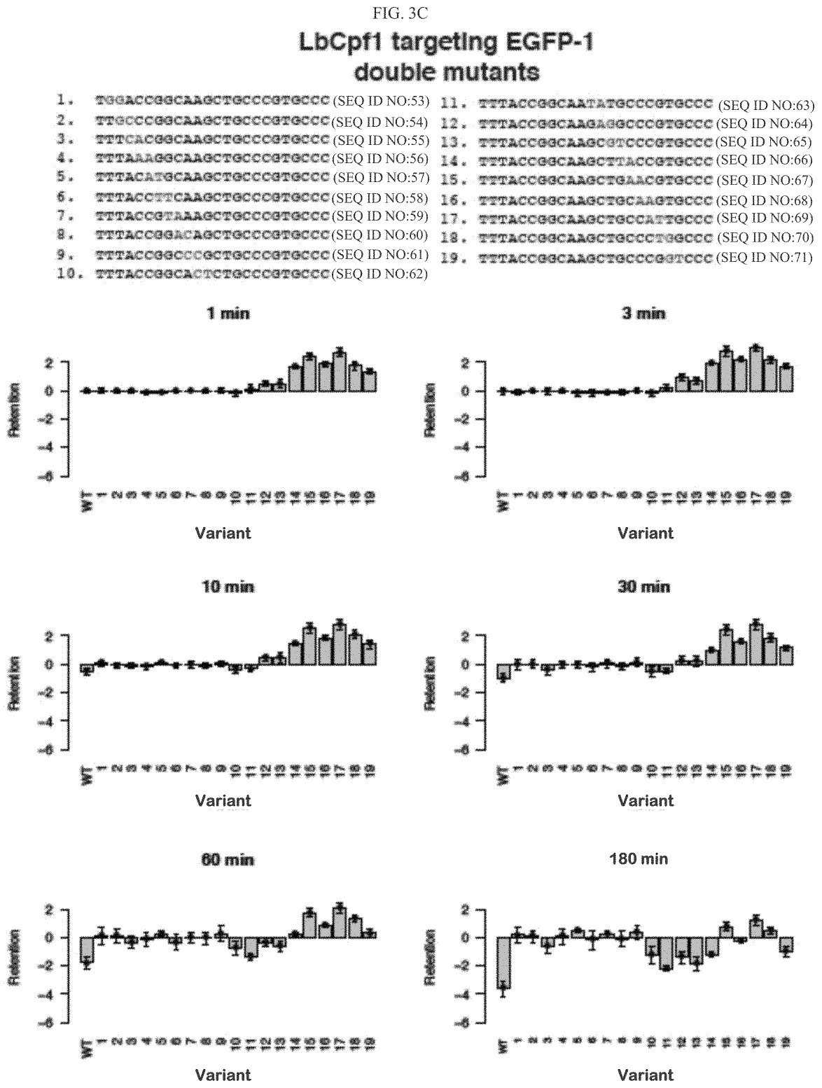

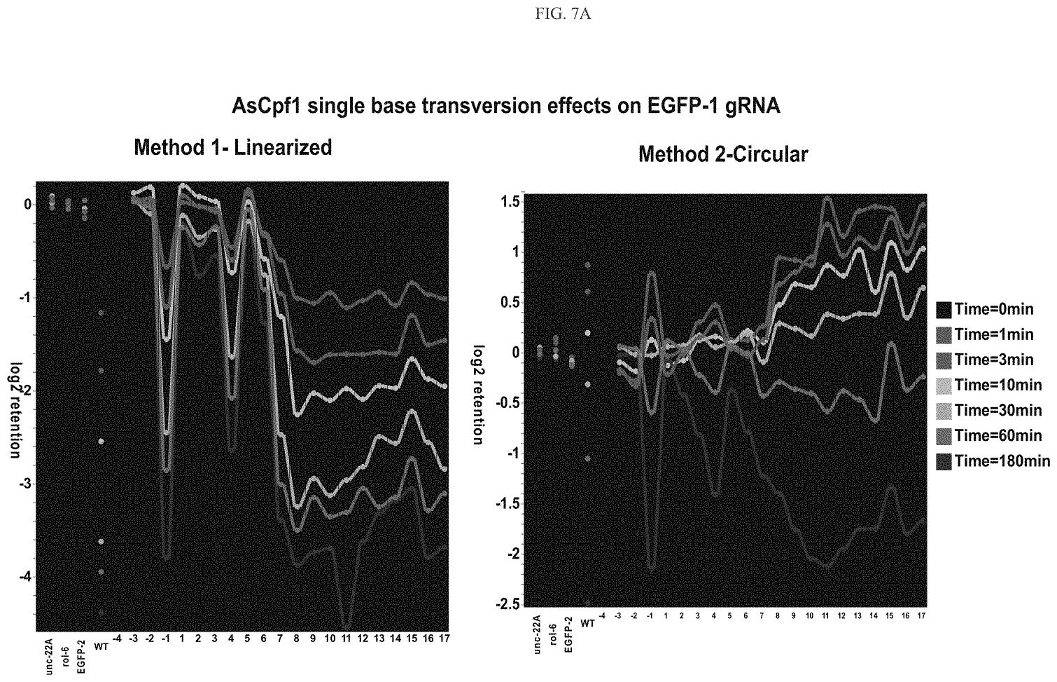

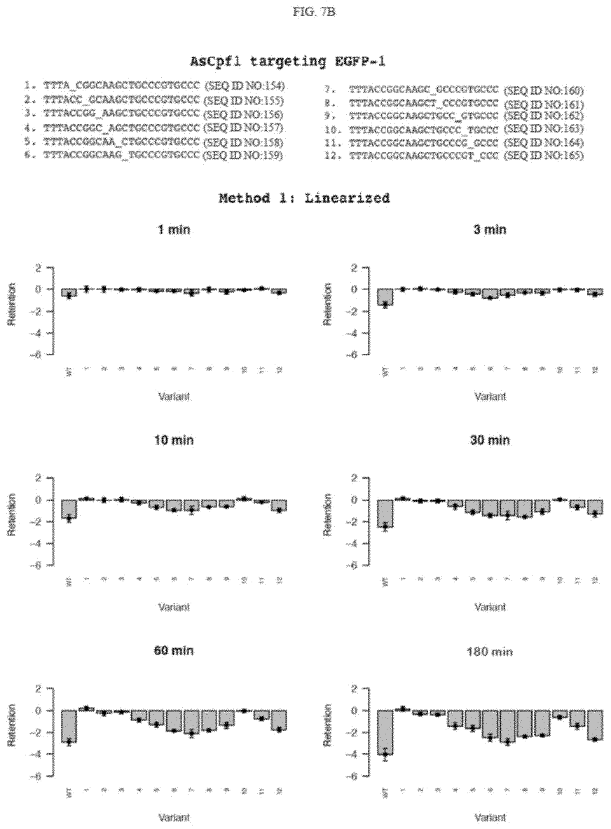

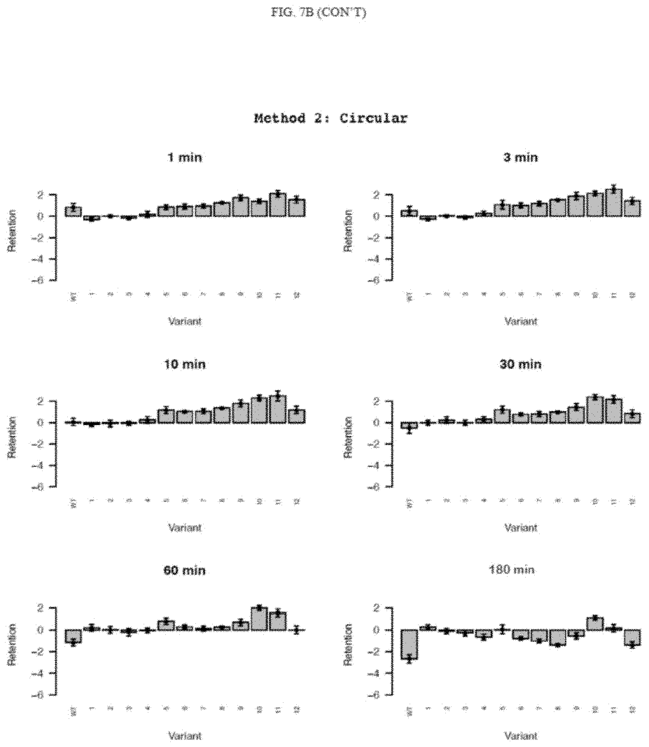

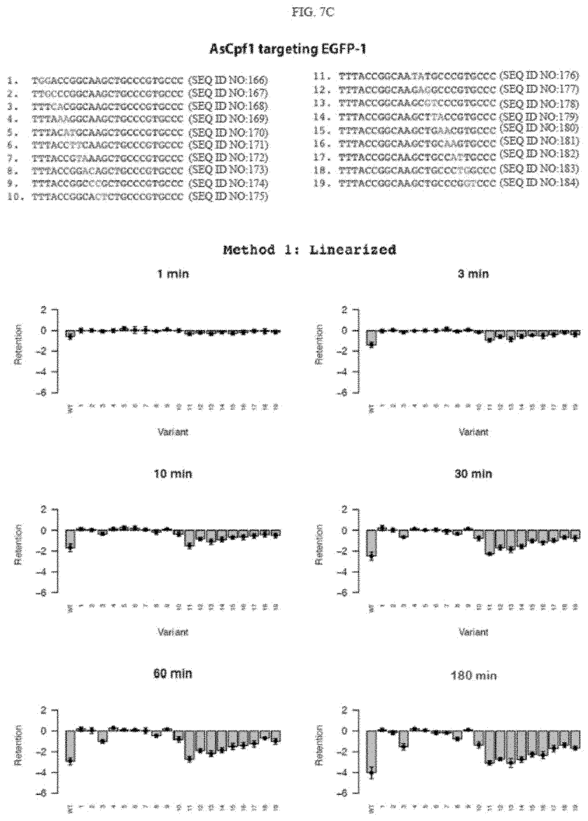

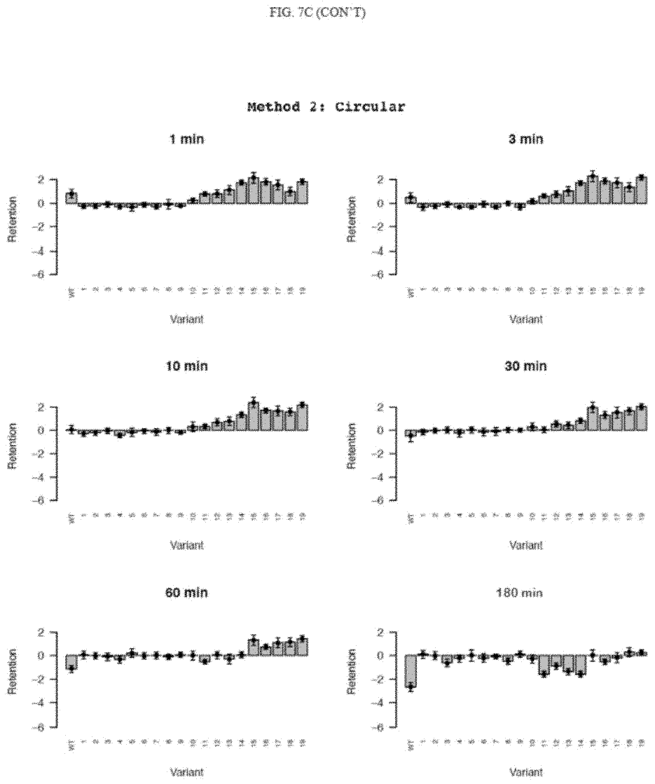

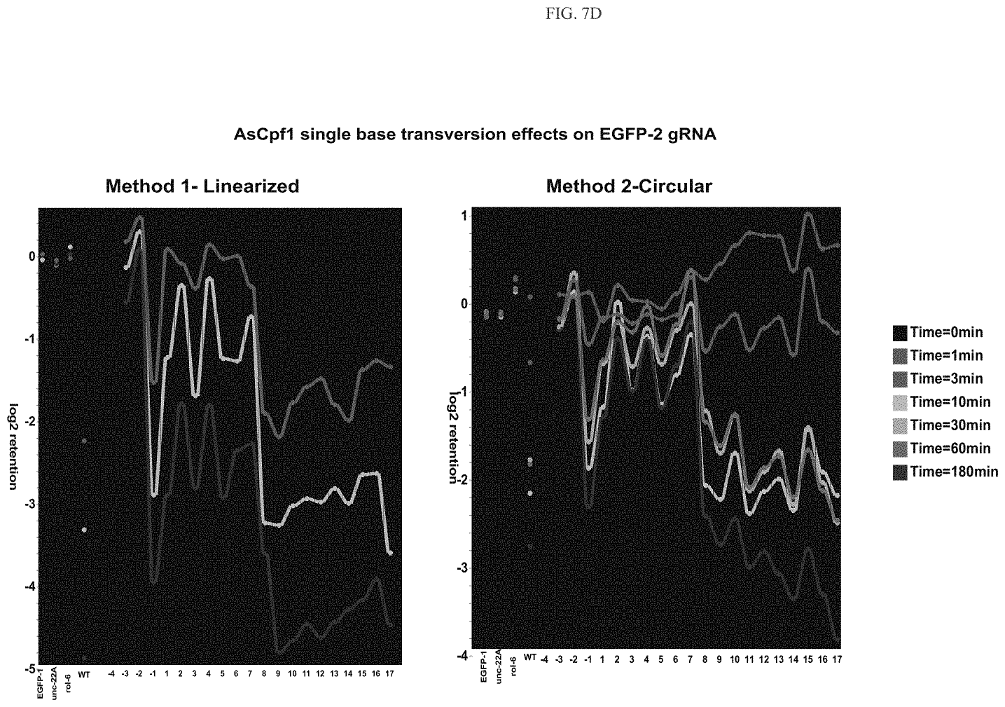

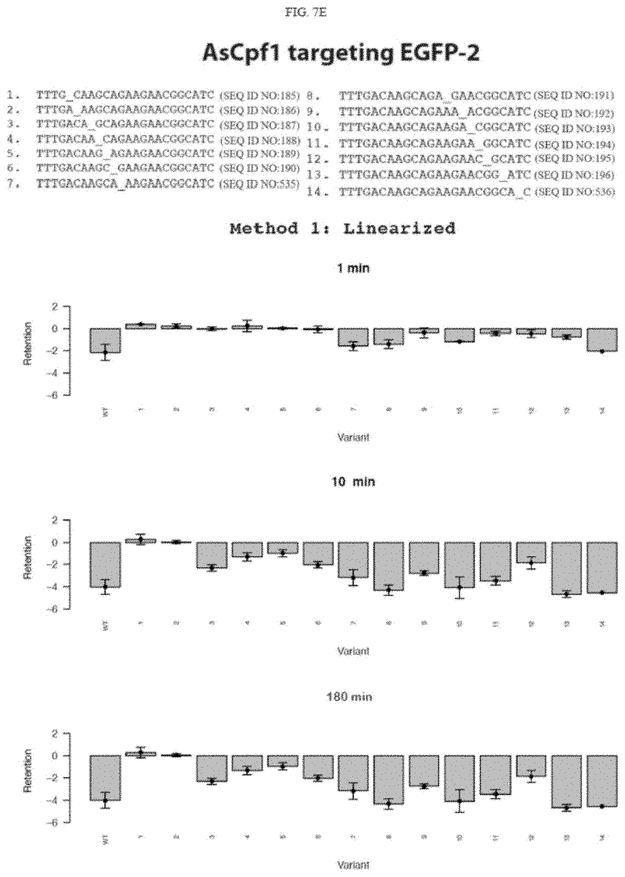

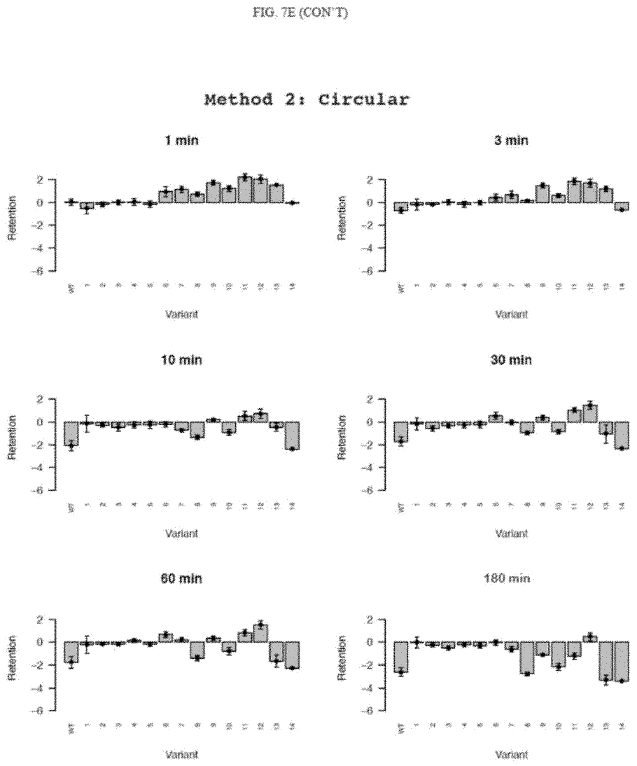

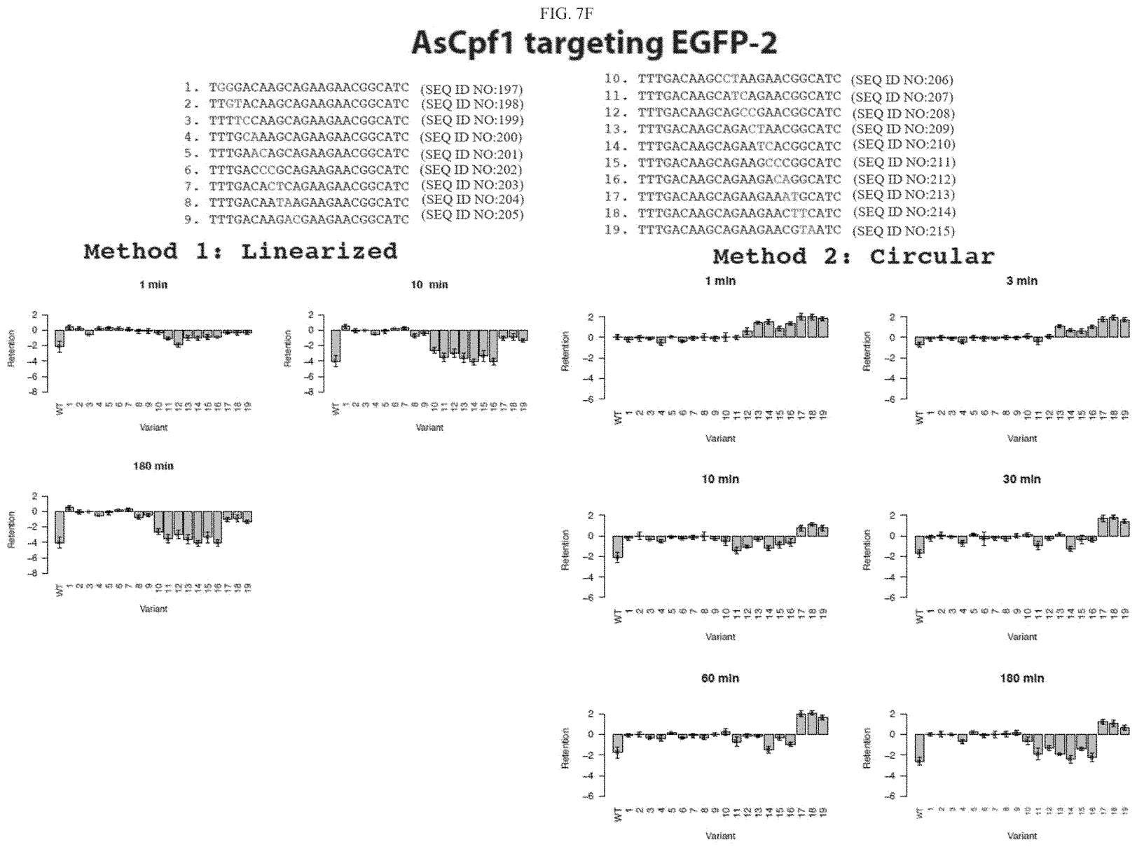

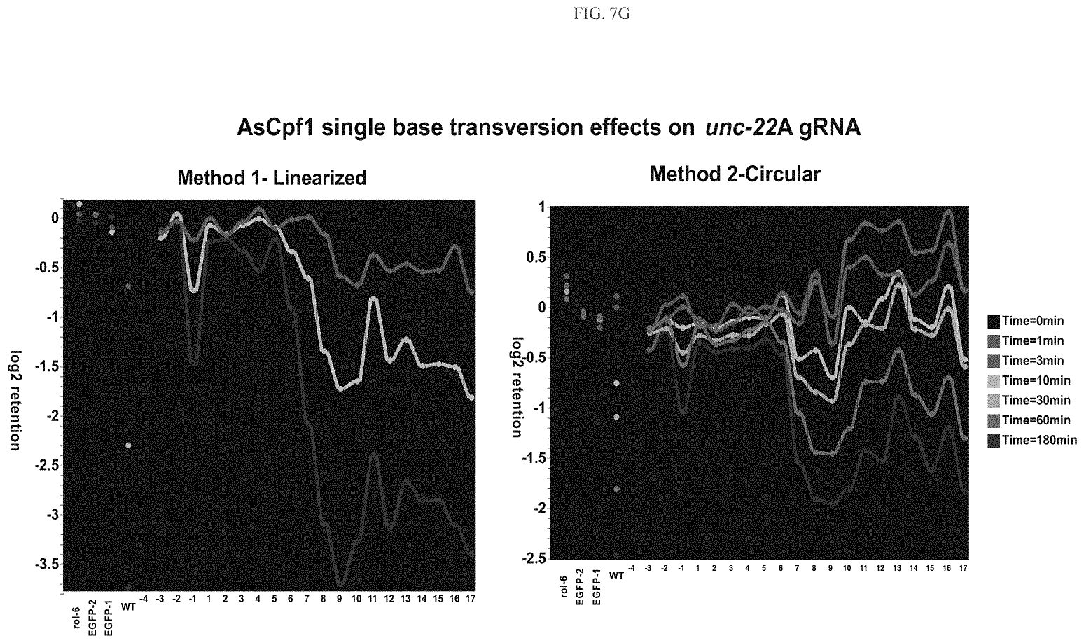

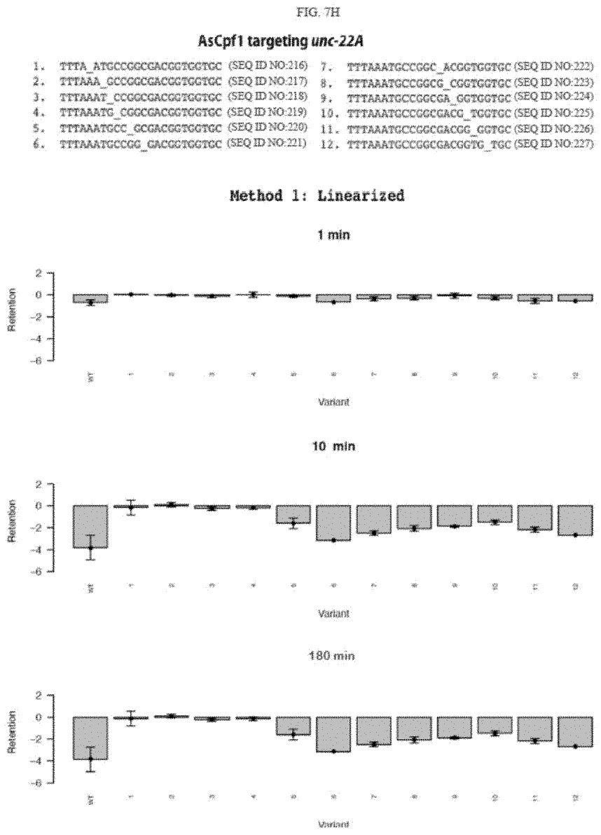

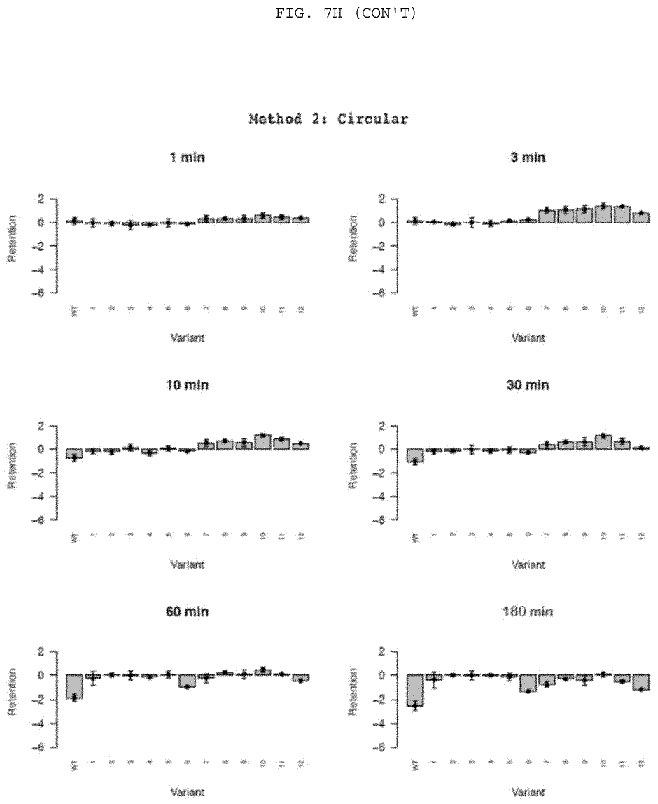

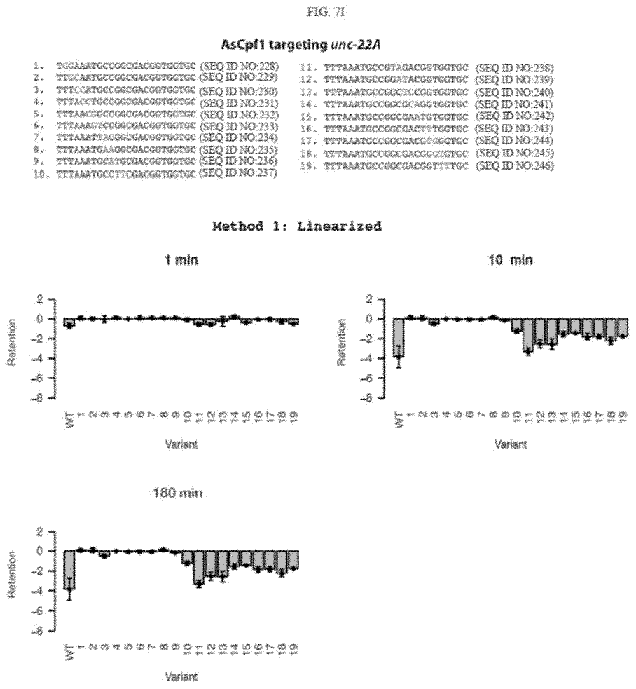

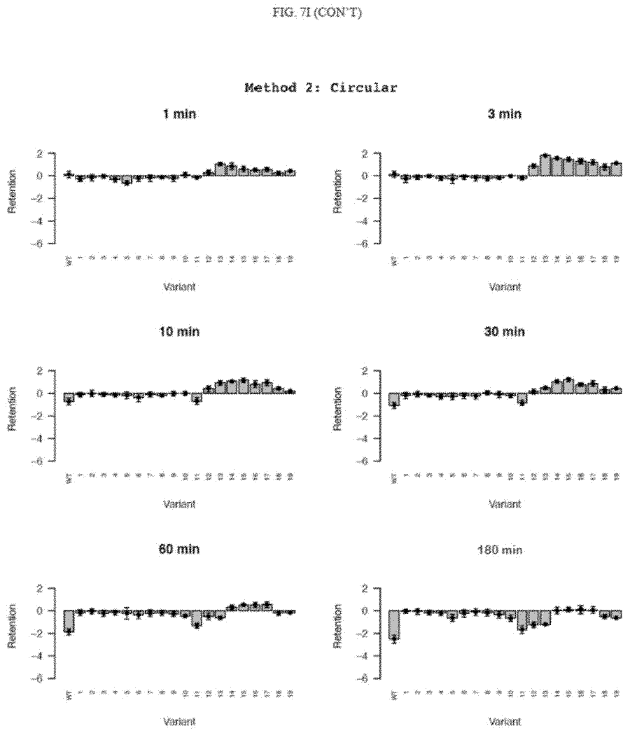

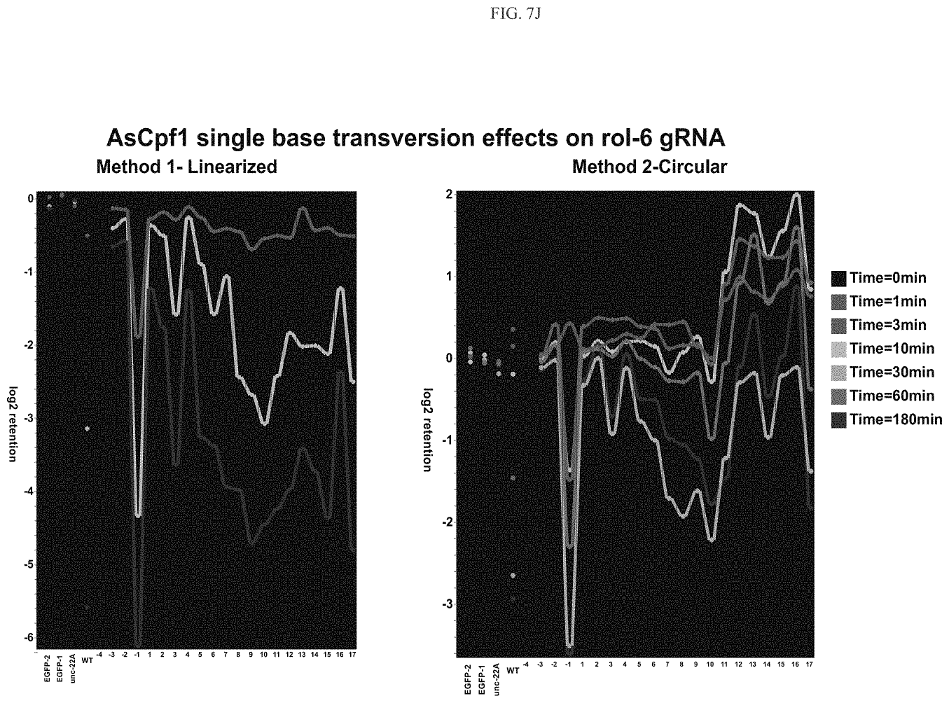

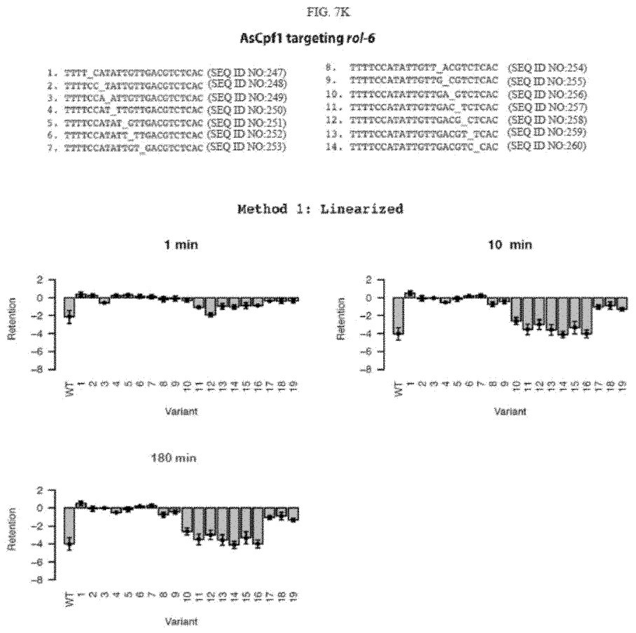

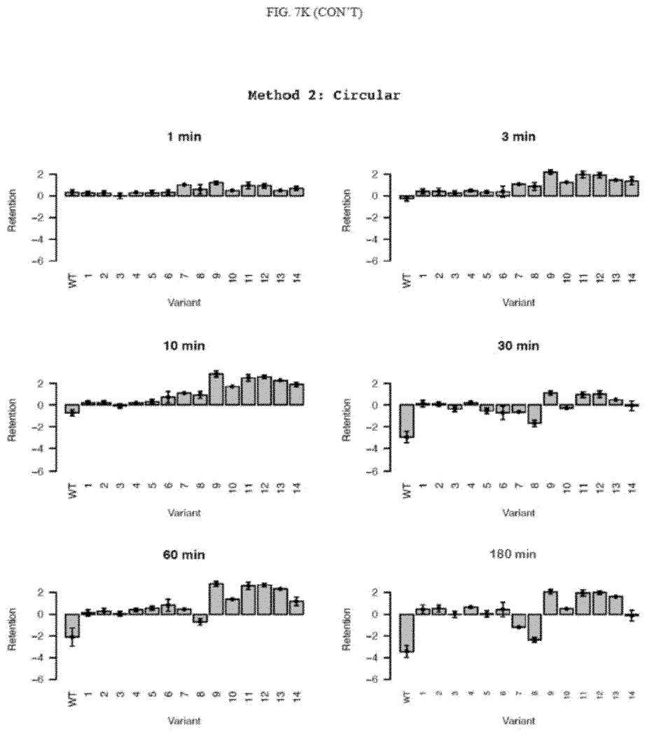

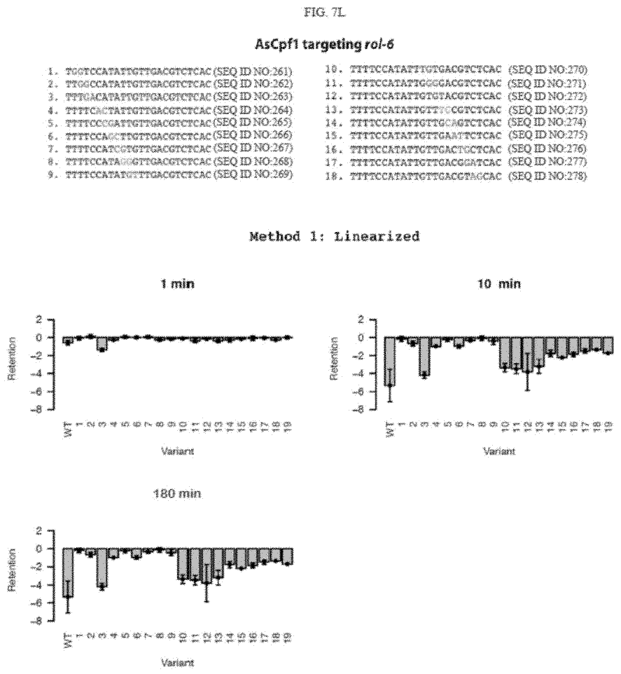

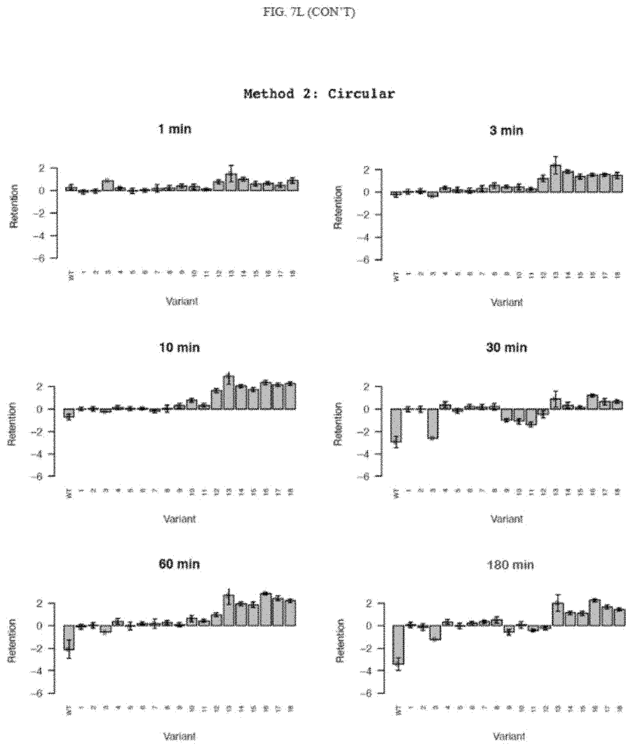

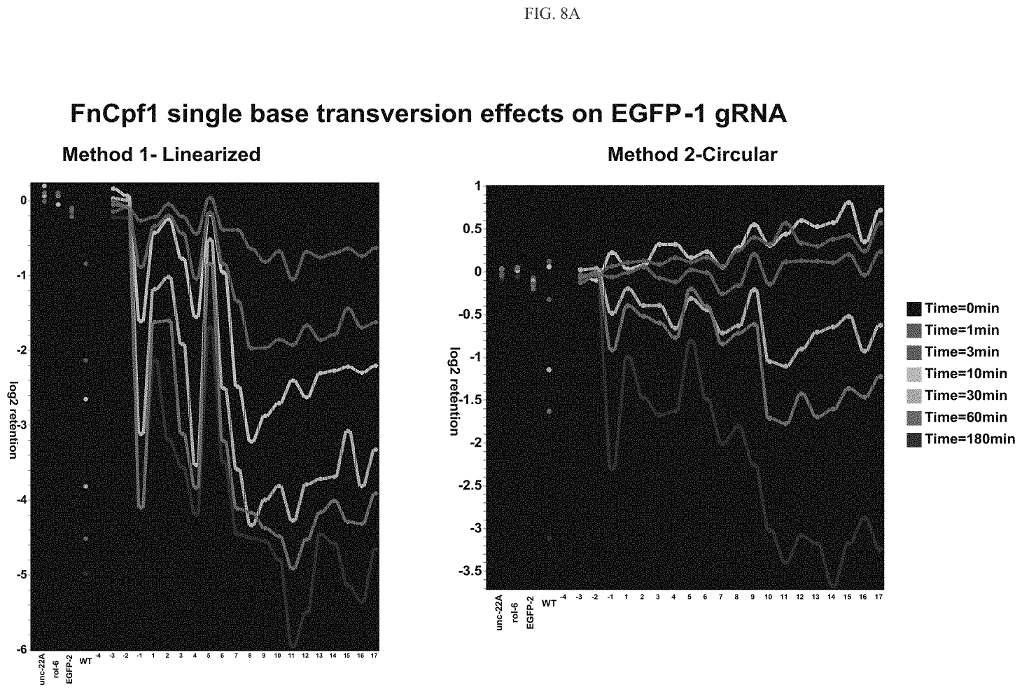

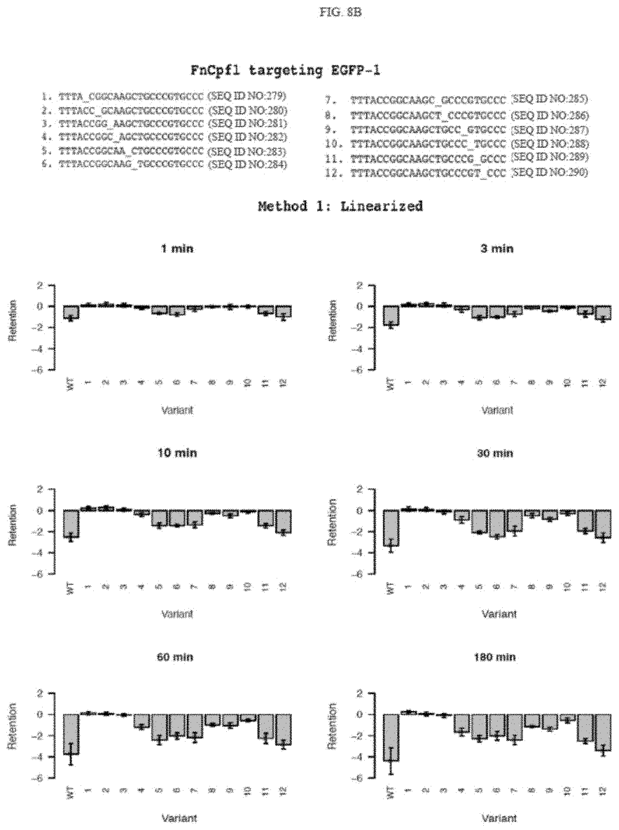

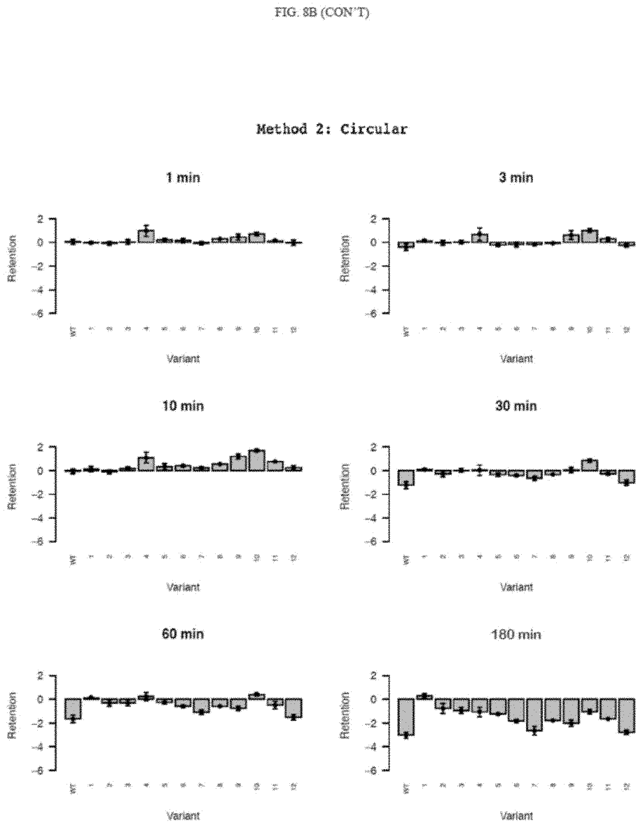

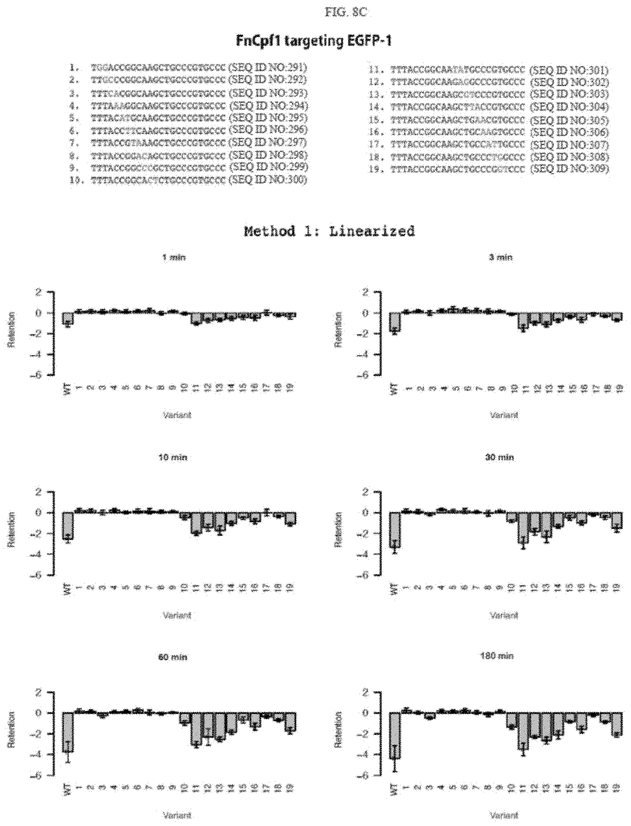

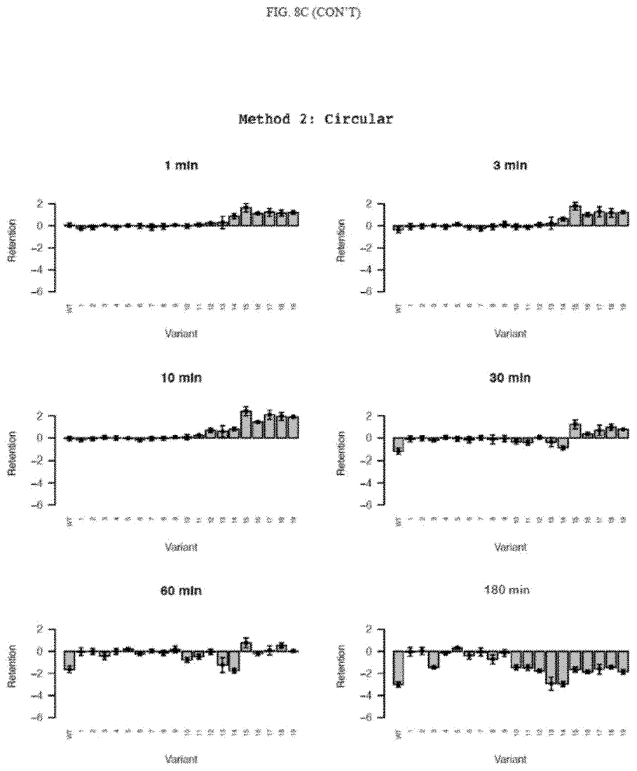

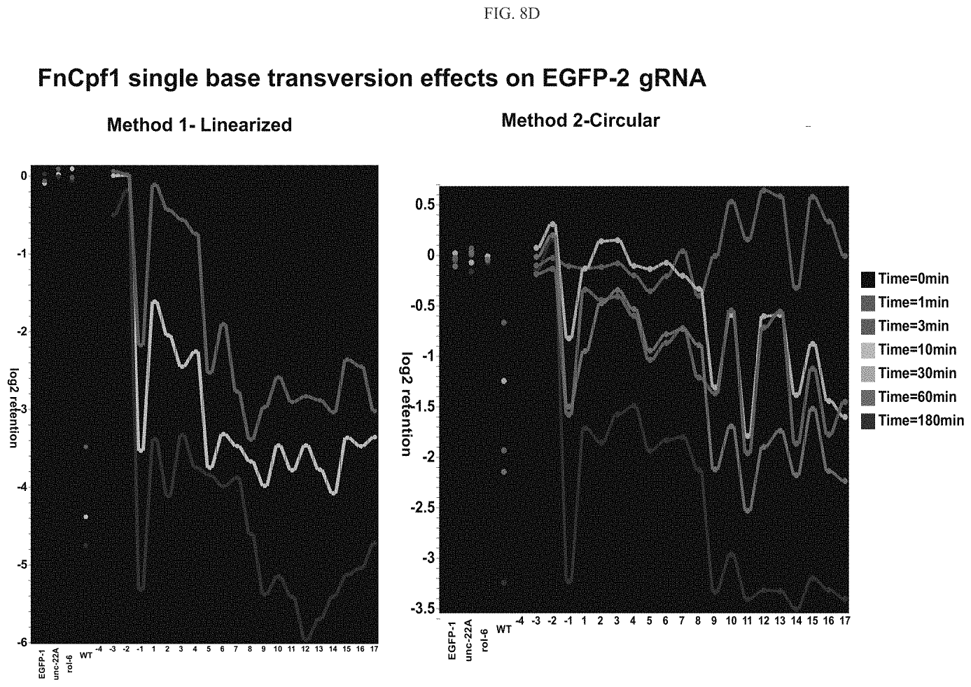

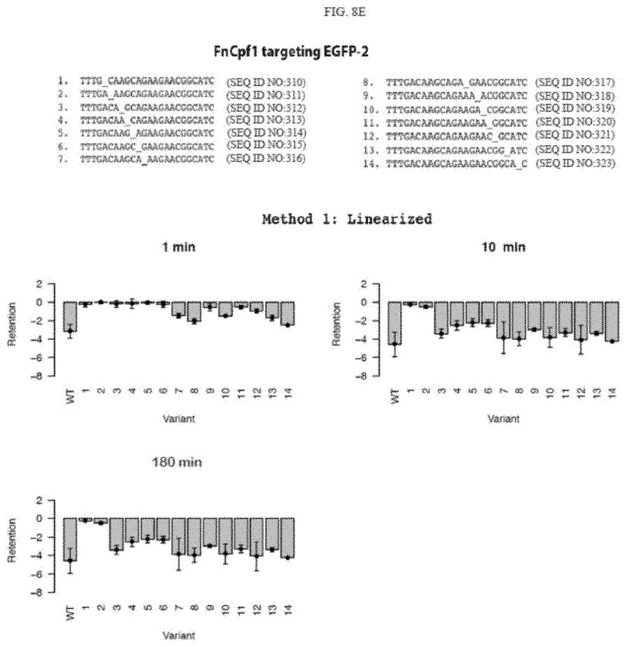

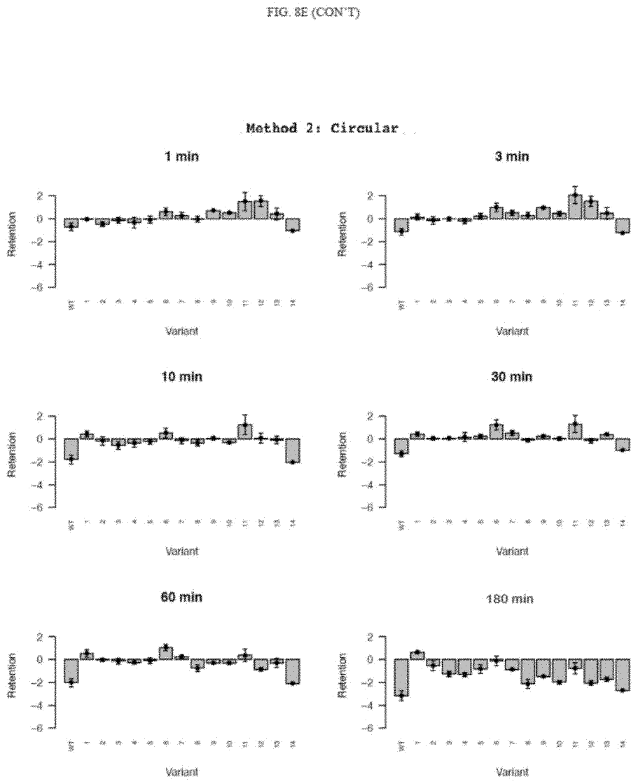

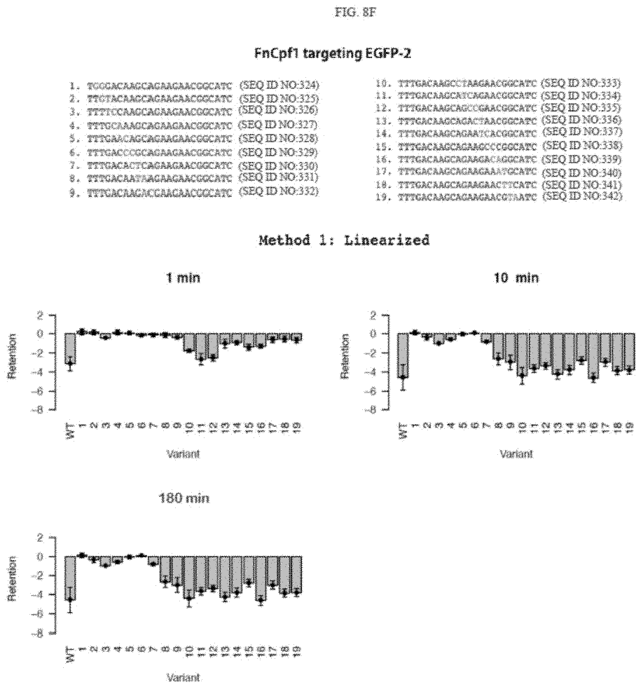

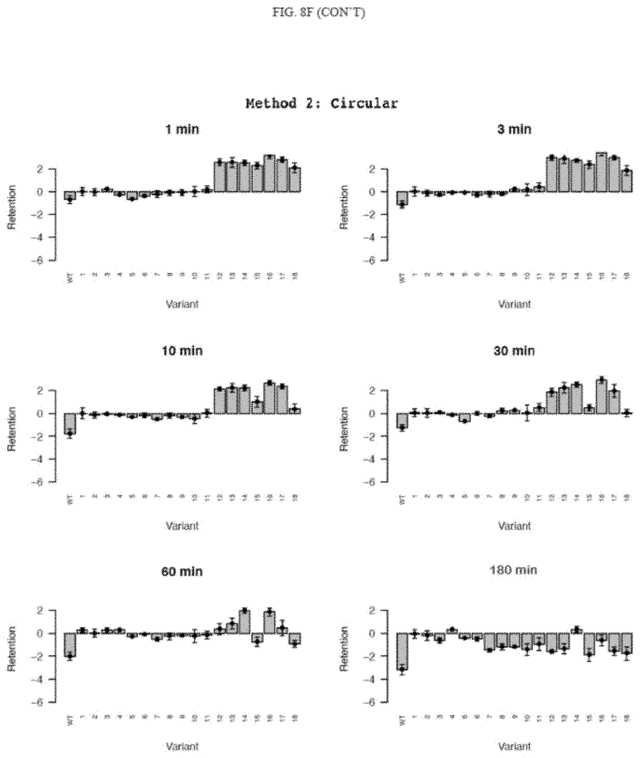

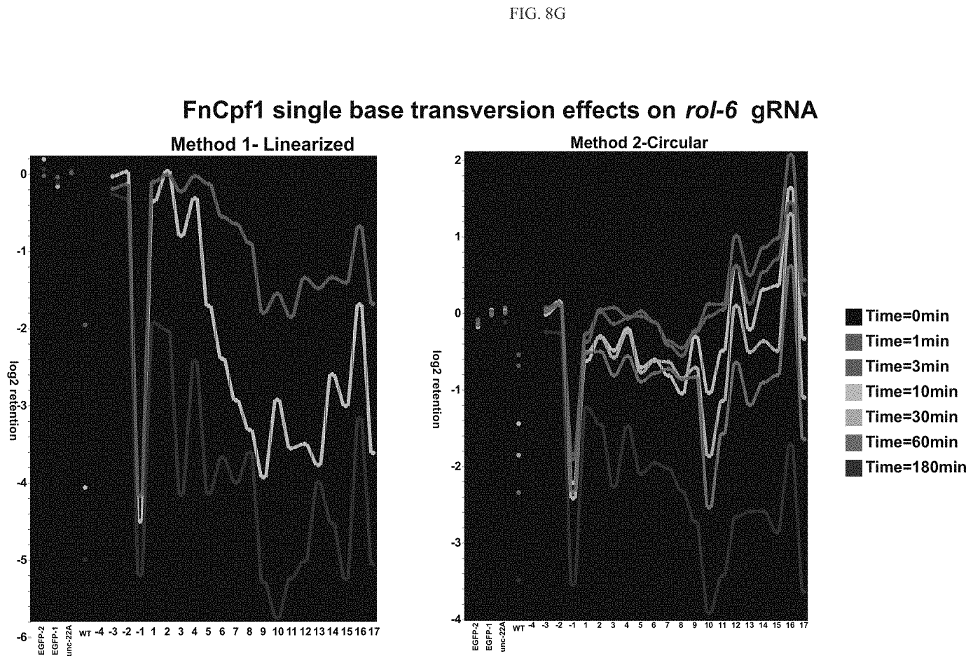

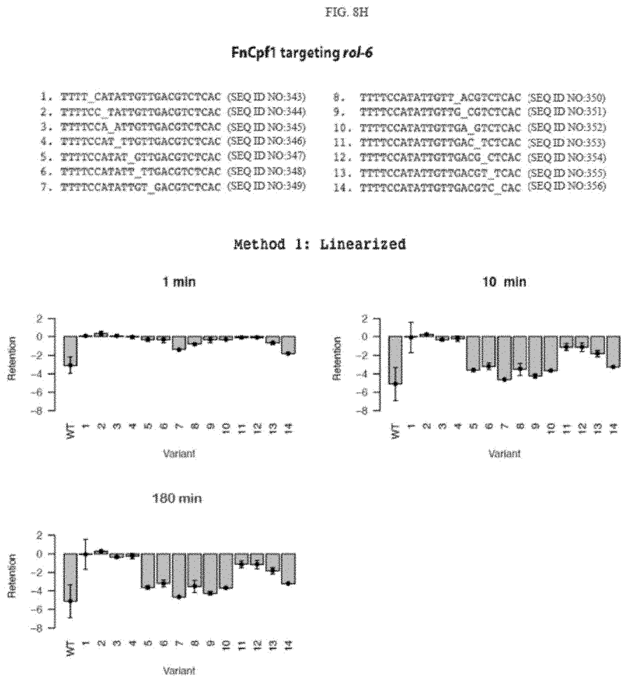

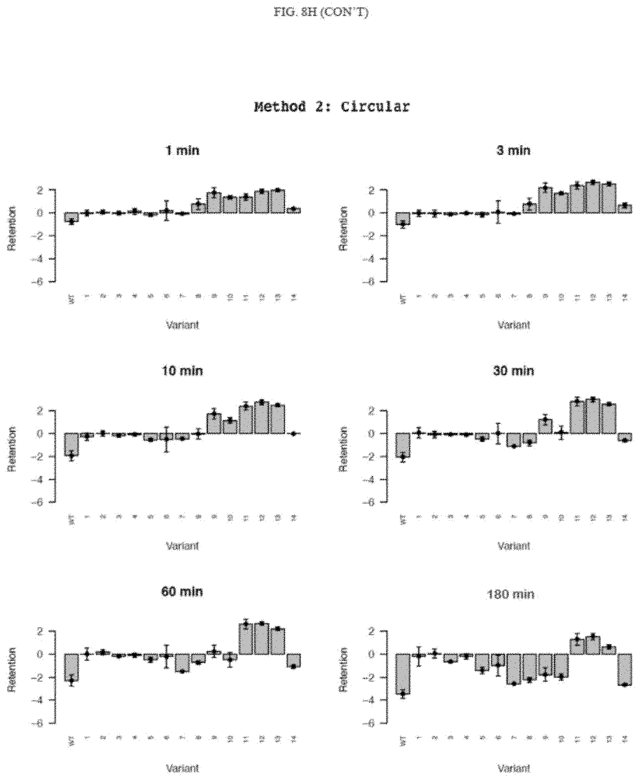

An unexpected feature of Cpf1 cleavage was observed in assays with the circular variant library in which no linearization was carried out before assessing retention (Method 2). Certain target site variants had highly positive retention scores, which suggest these variants increase (rather than decrease) their representation during a short-time cleavage reaction. Log-retention scores were observed as high as ˜3 (i.e., 2³ or 8-fold enrichment) for double mutants from time points 1, 3, 10, and 30 minutes (LbCpf1: FIG. 3C; AsCpf1 and FnCpf1: FIGS. 7A-7L and 8A-8I). For some targets, these effects disappeared at longer reaction times, as expected if there was eventual cleavage of the circular targets (FIGS. 3A-3C). The enhanced recovery led to the hypothesis of a rapid nicking of the initial substrate followed by a relatively slow cleavage on the opposite strand. This would explain the observed over-representation after addition of Cpf1 and highly positive retention score followed by much slower loss of the observed retention.

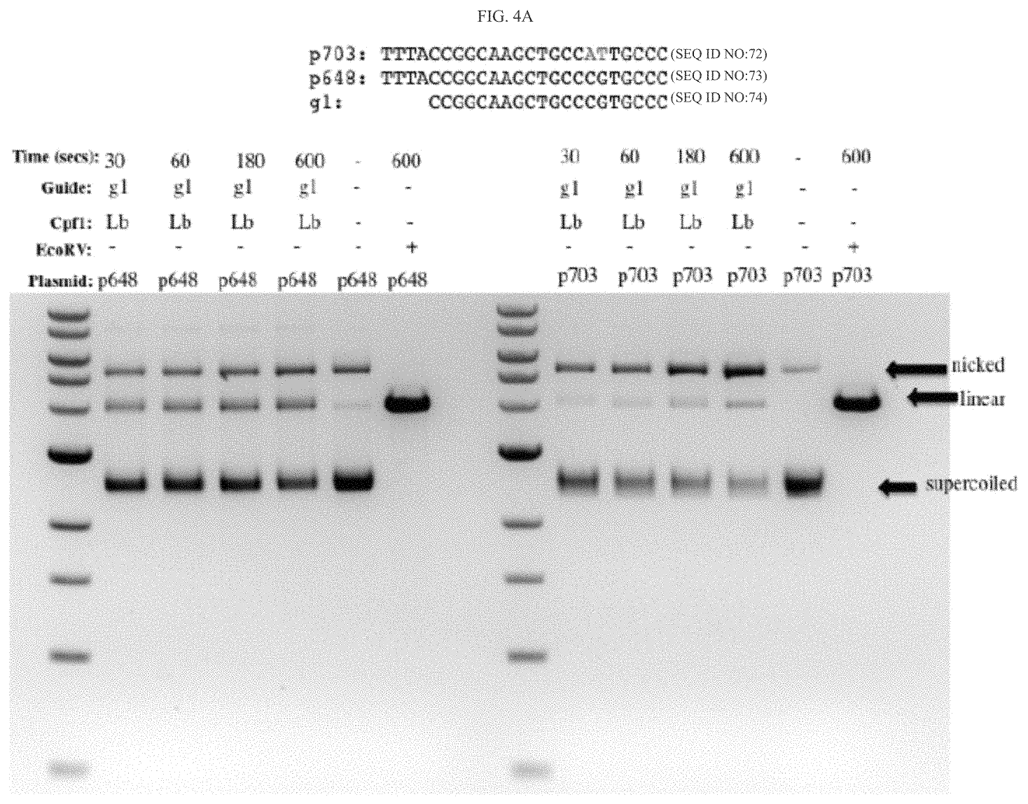

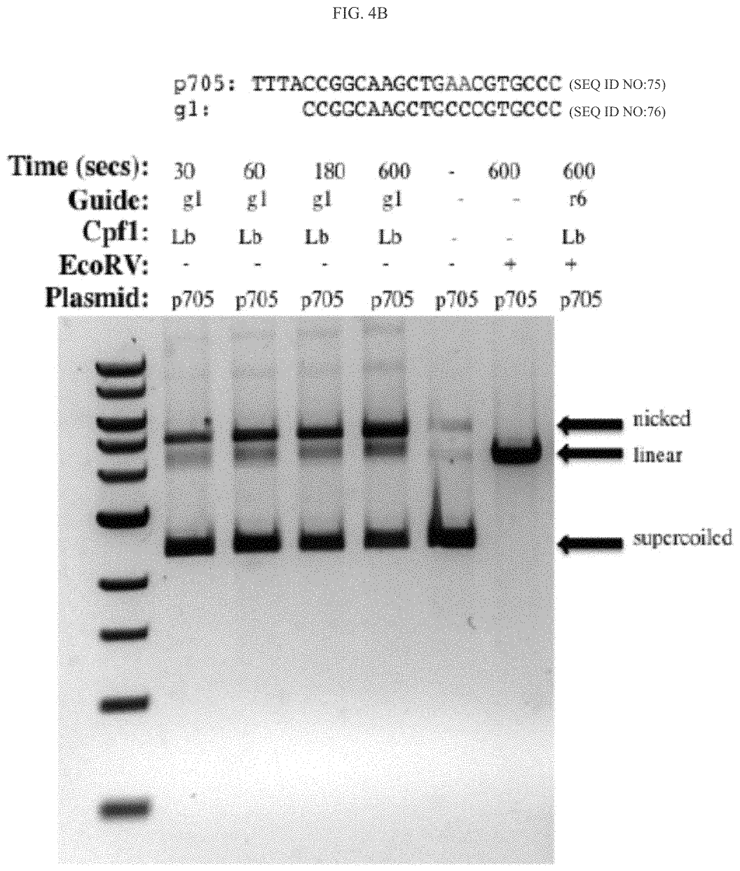

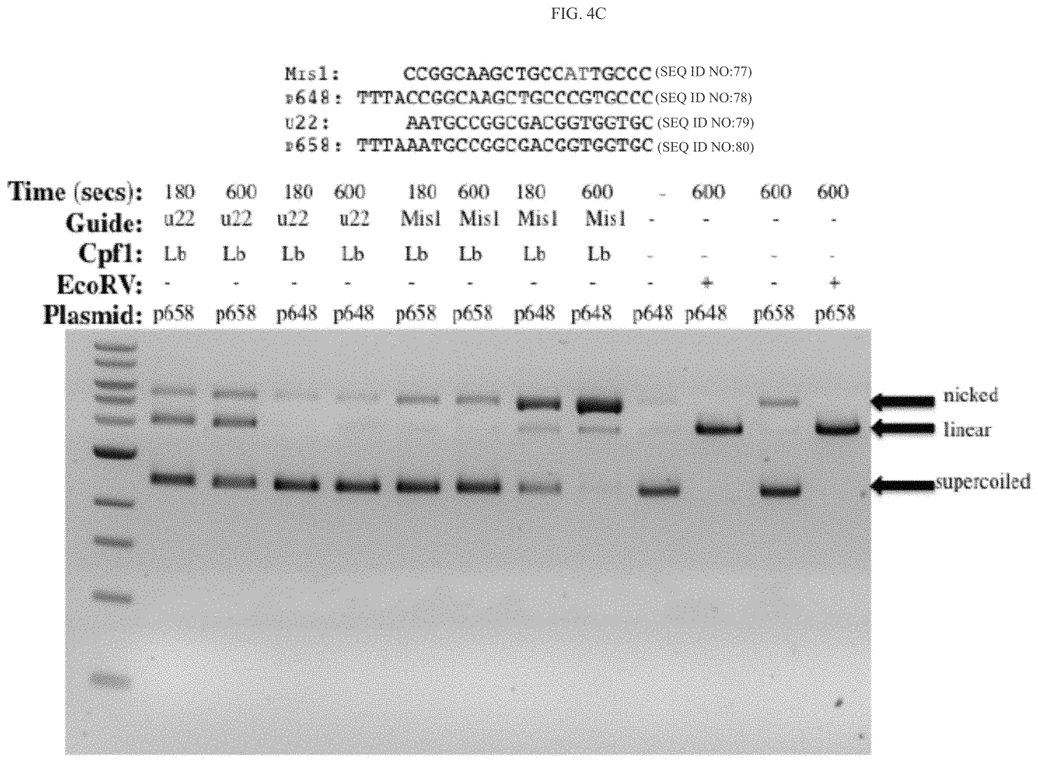

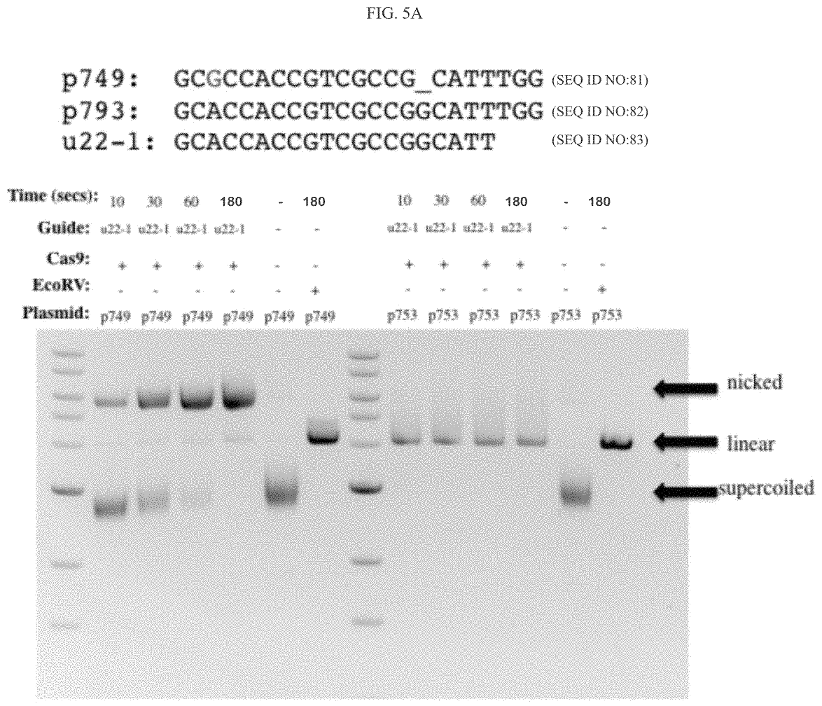

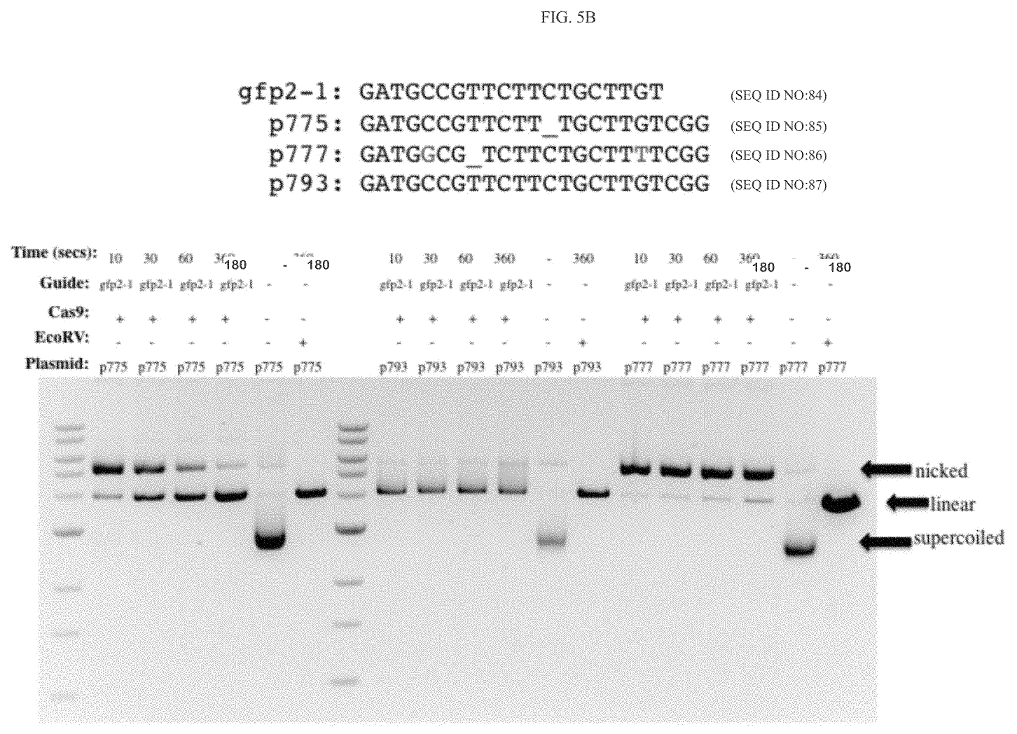

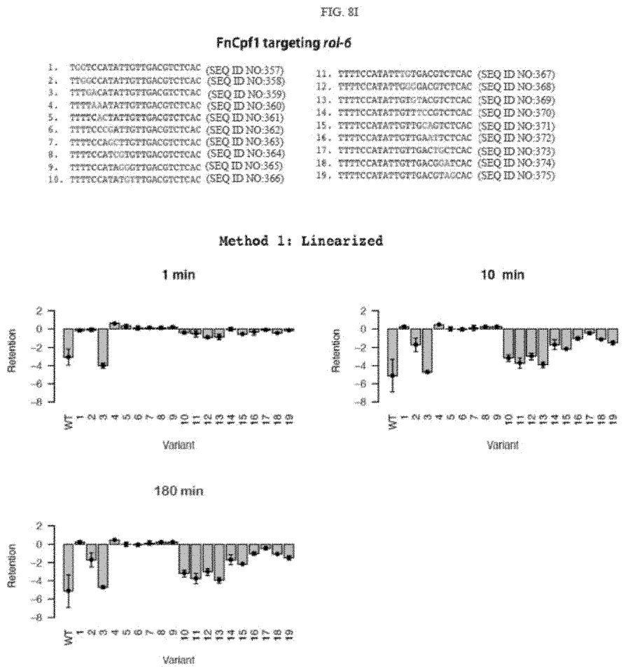

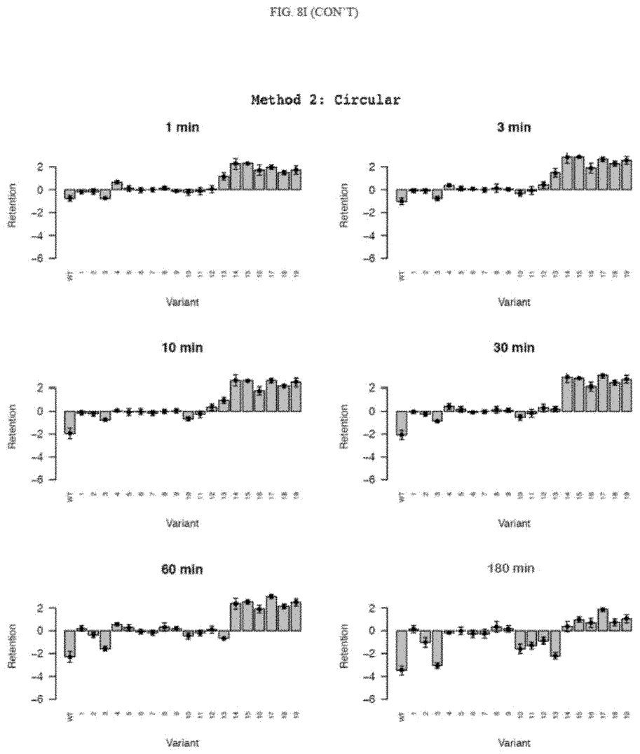

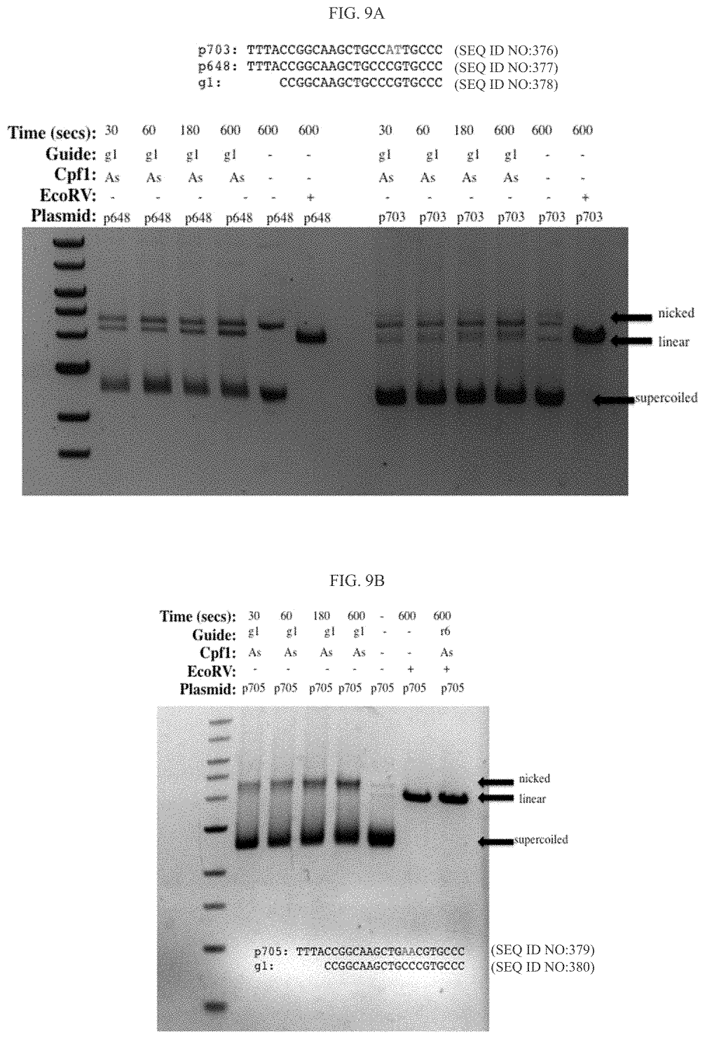

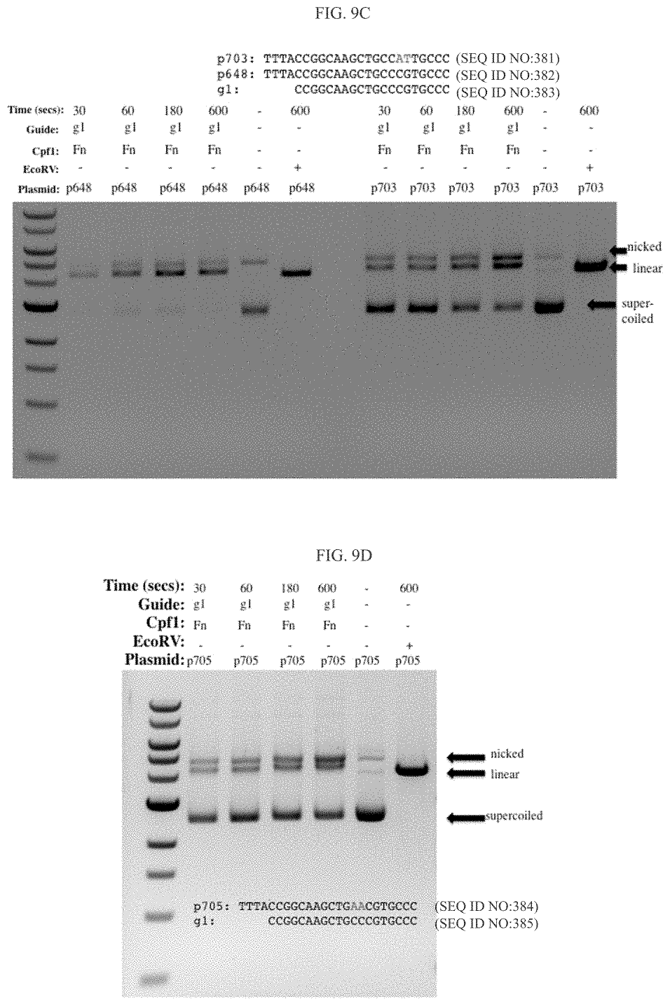

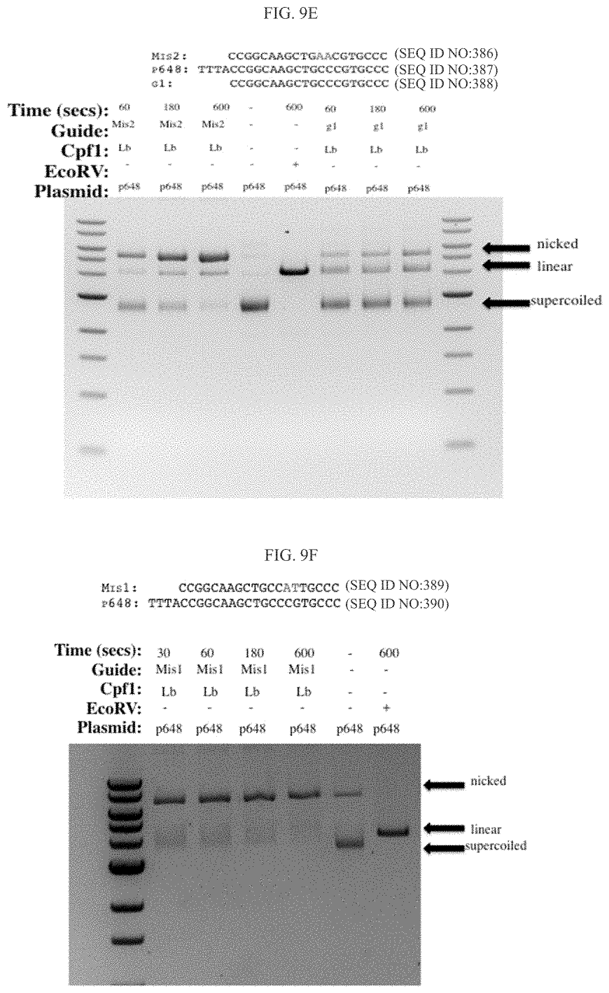

The nicking hypothesis was tested by reacting Cpf1::gRNA complexes with individual plasmid substrates and examining topology using ethidium-containing native agarose DNA gels. Sequence variants with highly positive retentions were identified in the high throughput data and either cloned into a DNA plasmid or synthesized as a gRNA. Using a DNA target with double-consecutive transversion mutations in positions 12-13 and 14-15 in WT EGFP-1 gRNA, it was found that LbCpf1 was rapidly capable of converting closed circular to nicked circular substrates in these assays (FIGS. 4A and 4B). In addition, the nicking ability was found to be specific, with no observed nicking of substrates lacking homology to the gRNA (FIG. 4C). The nicking ability was confirmed in AsCpf1 and FnCpf1 with their respective EGFP-1 gRNAs (FIGS. 9A-9D).

To evaluate the determinants for nicking and cleavage, a number of reciprocal experiments were carried out in which mutated gRNAs were used in assays with wild-type targets. These assays showed nicking and some linearization, as expected if the ability to form a nicking enzyme is a general feature of certain classes of guide::target mismatch. There was some non-equivalence in the target-mutated versus guide-mutated assays, depending on the individual gRNAs (FIGS. 9E and 9F). These data indicate an interaction in which specific sequence along with the pattern of mismatches determine the balance between nicking and cleavage activities.

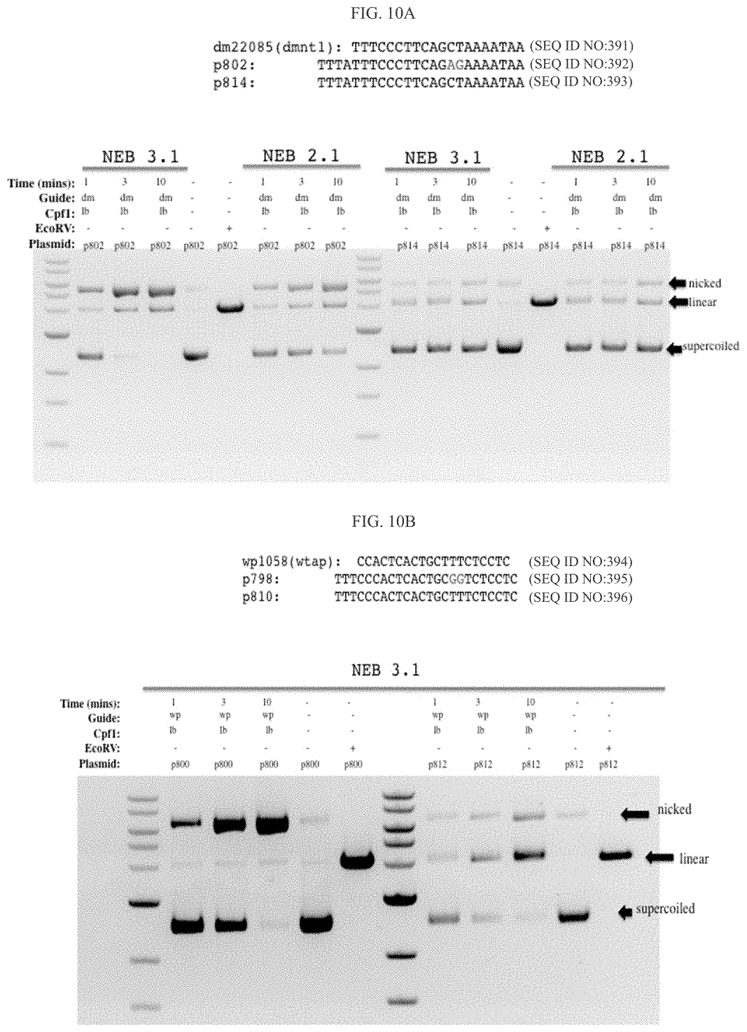

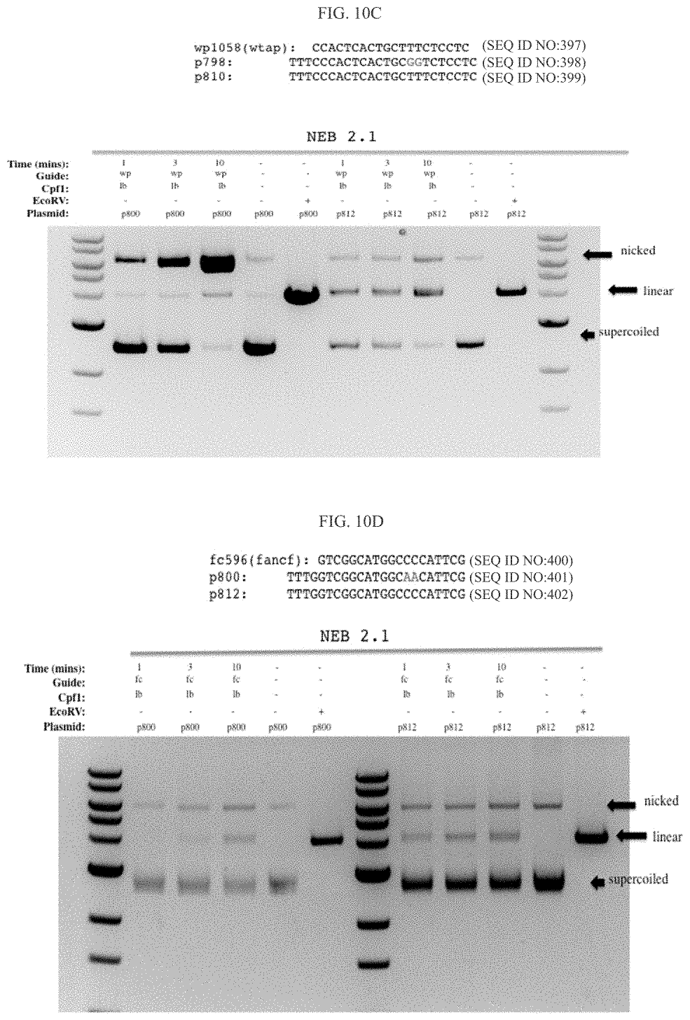

The above observations raise the possibility that an appropriately designed gRNA might produce effective single-strand nicking activity on an arbitrary substrate. Some possible guidelines for such design are suggested from the patterns of mismatch effects on initial sequence recovery in FIGS. 4A-4C, 7A-7L, and 8A-8I). In particular, a strong tendency to nick for templates with a combination of transversion point mutations in the distal region (positions 9-15) was observed. Taking consecutive double mutation at positions 12 and 13 as a provisional lead for such assays, it was first tested the ability to produce nicking activities towards additional targets where no high throughput analysis of target specificity had been carried out (targets dm22085 (DNMT1), fc596 (FANCF) and wp1058 (WTAP)). While these three sequences show different degrees of cleavage for matched gRNA:target combinations, all three strongly bias toward nicking with the indicated double mutant target (FIGS. 10A-10D).

### Example 3. Cas9 Nicking and Cleavage Specificity

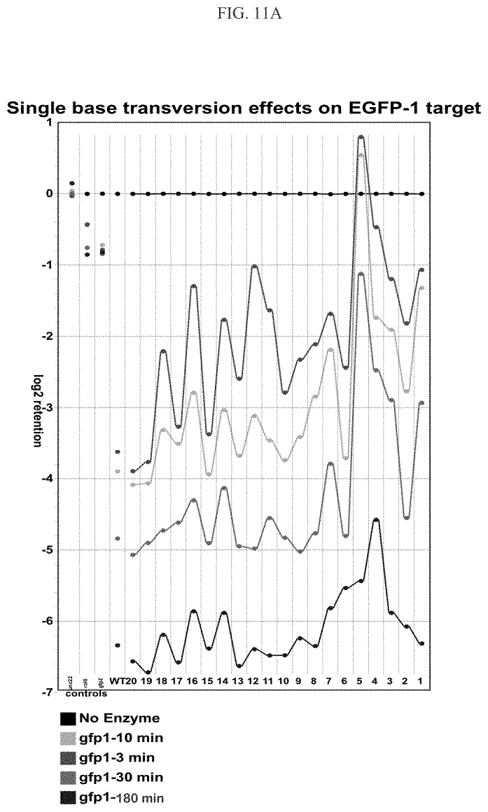

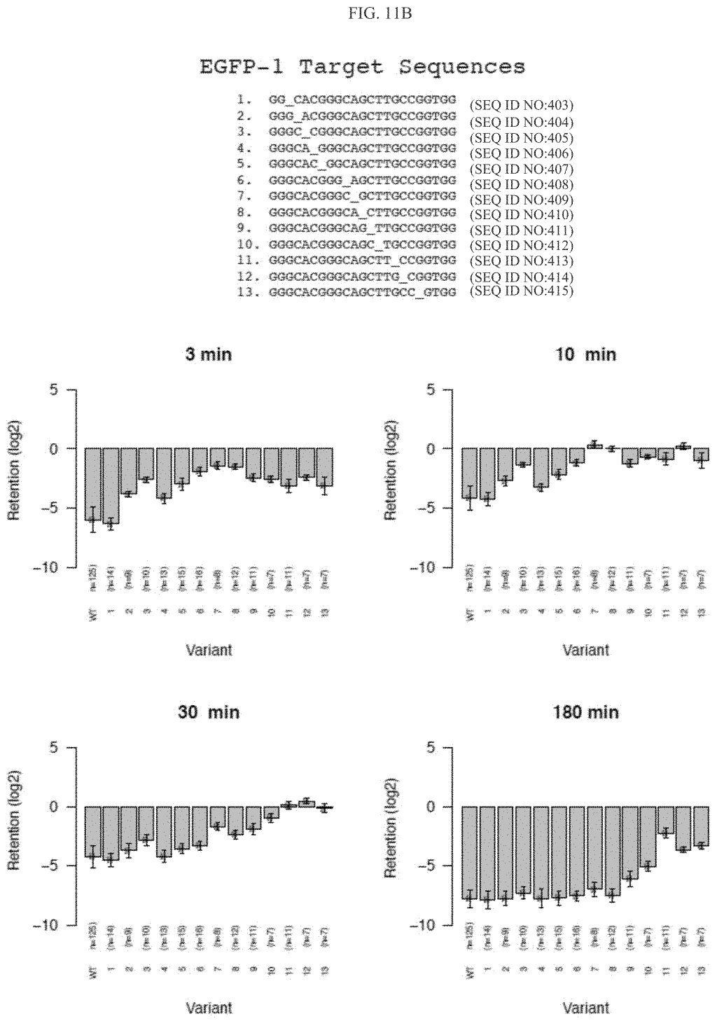

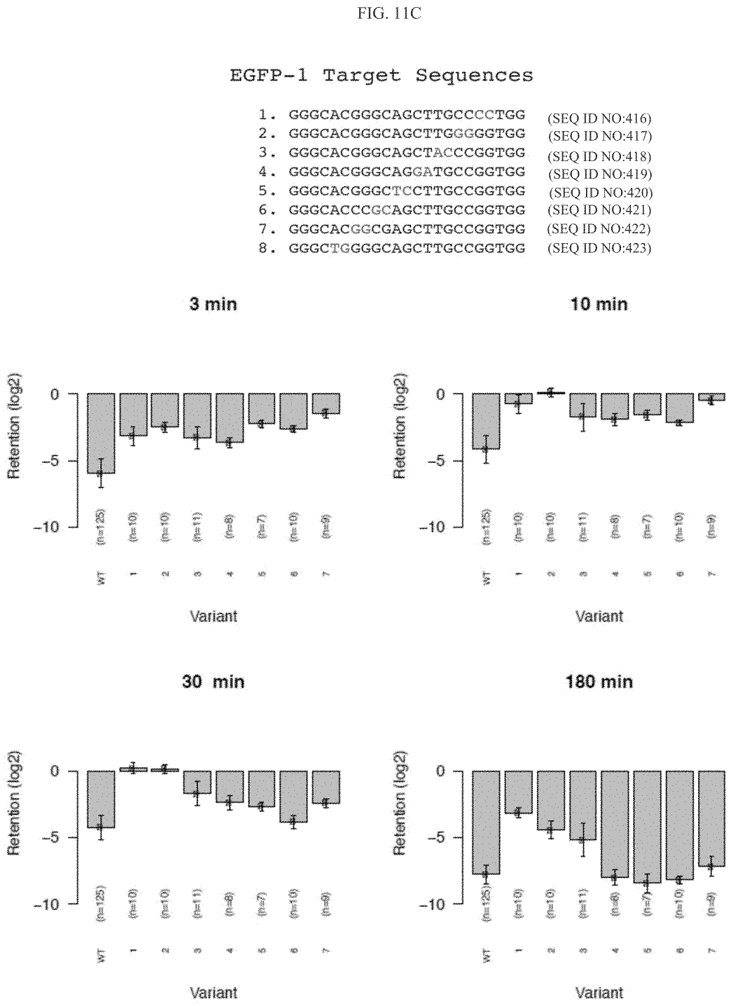

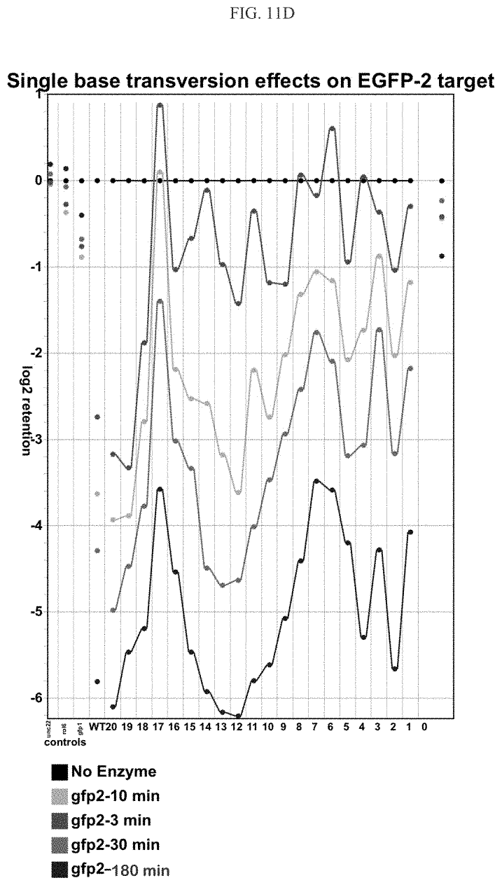

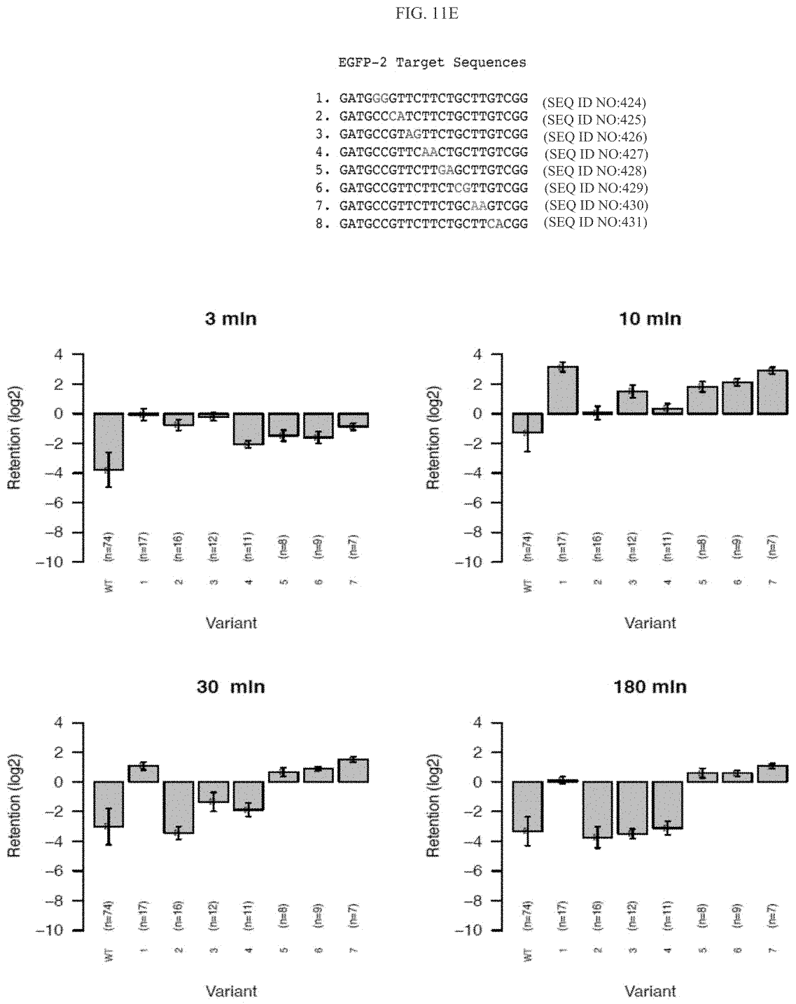

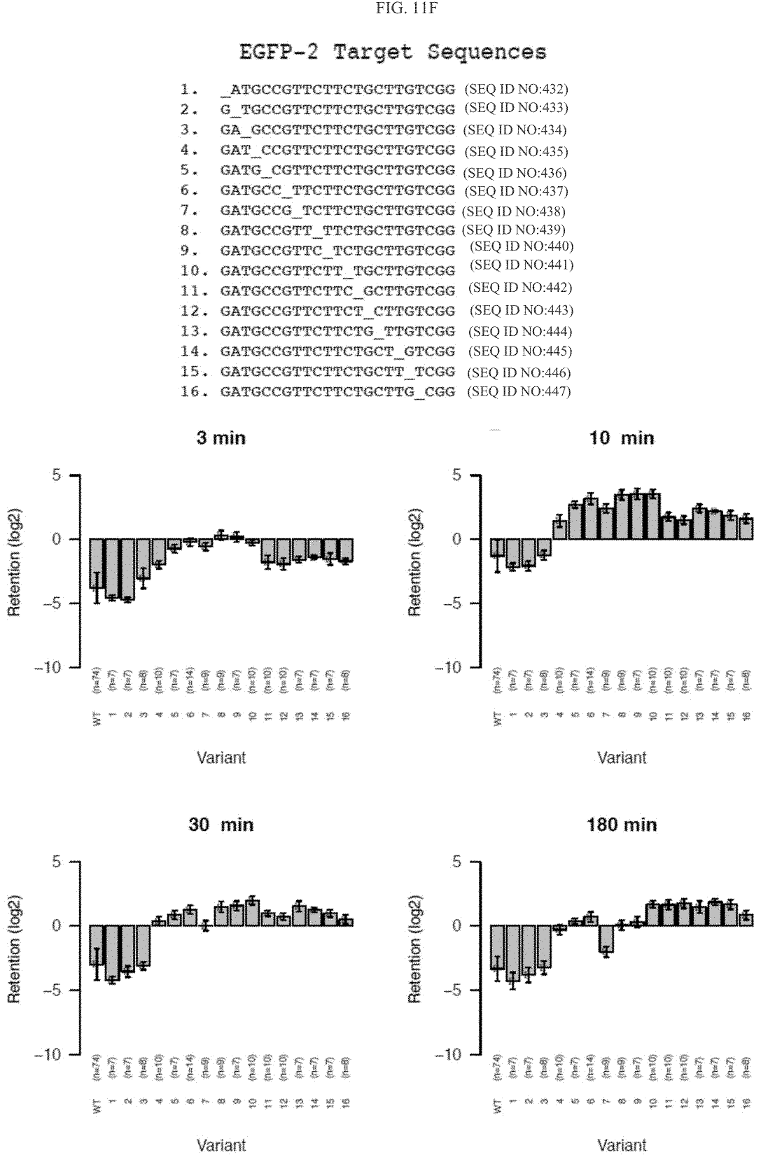

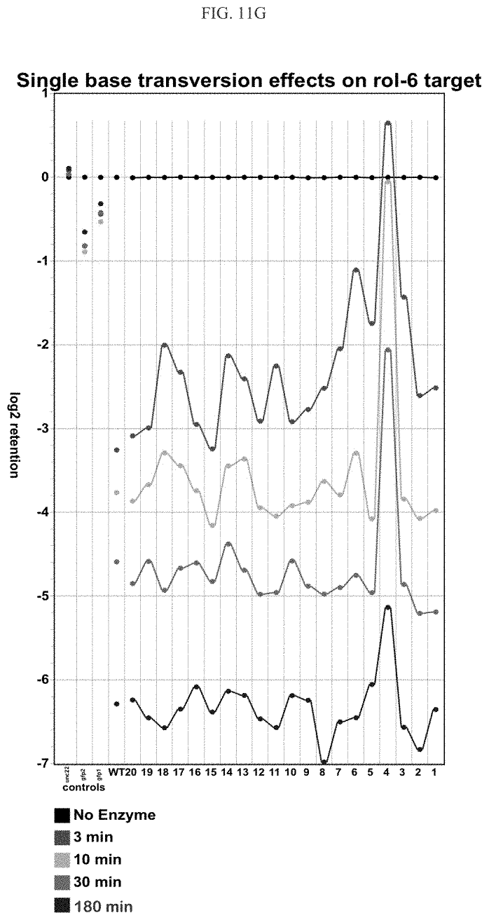

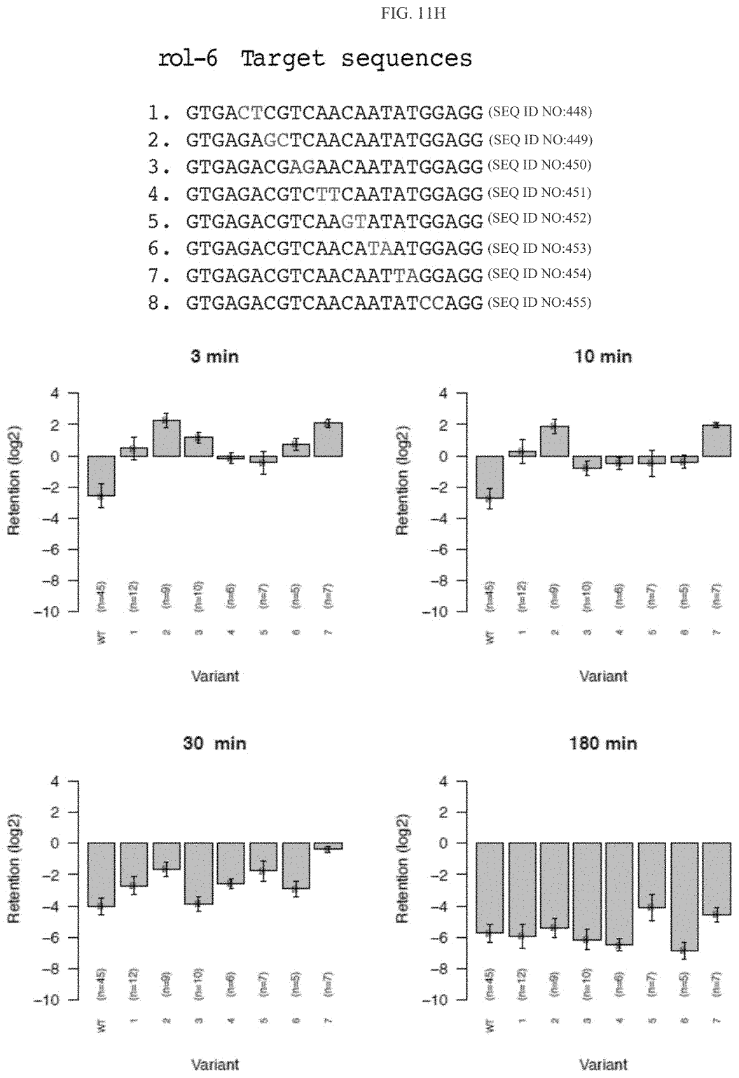

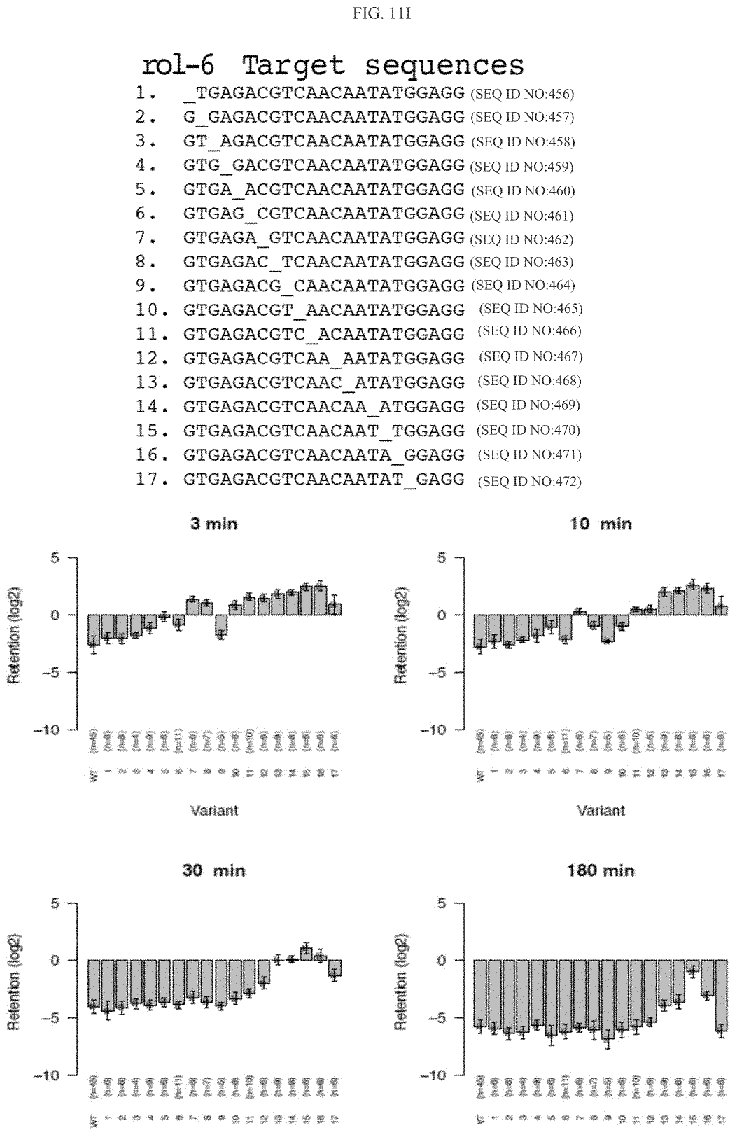

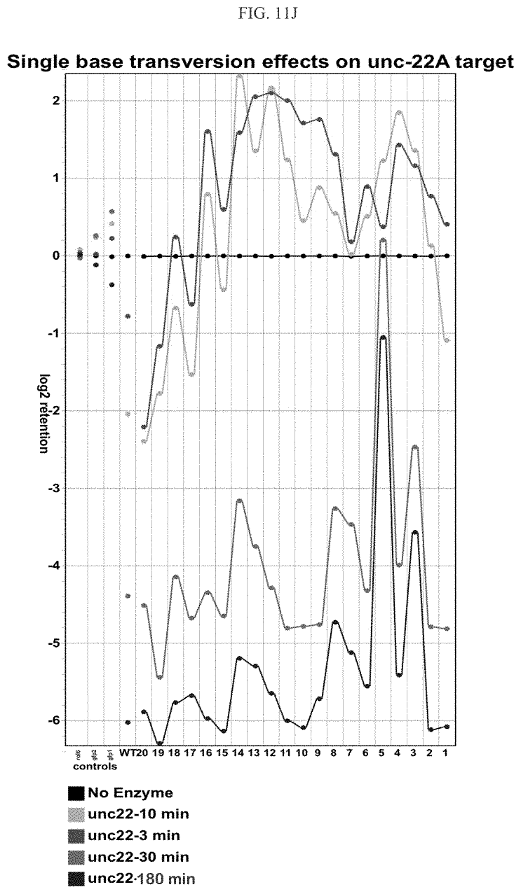

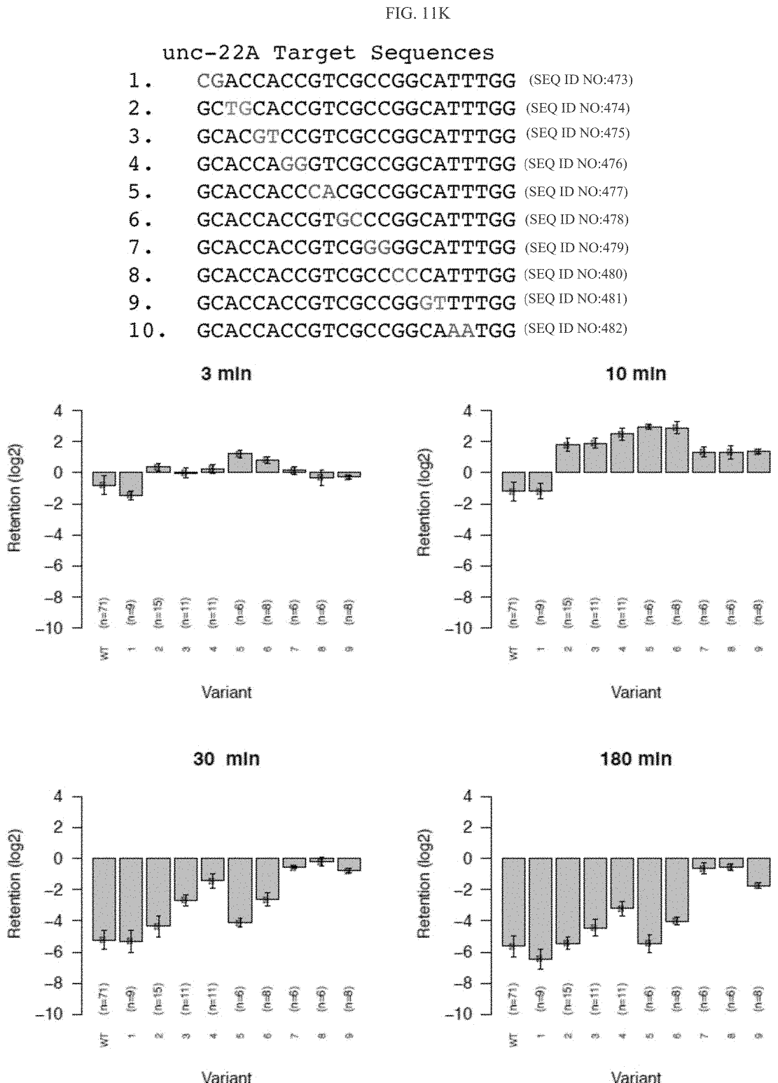

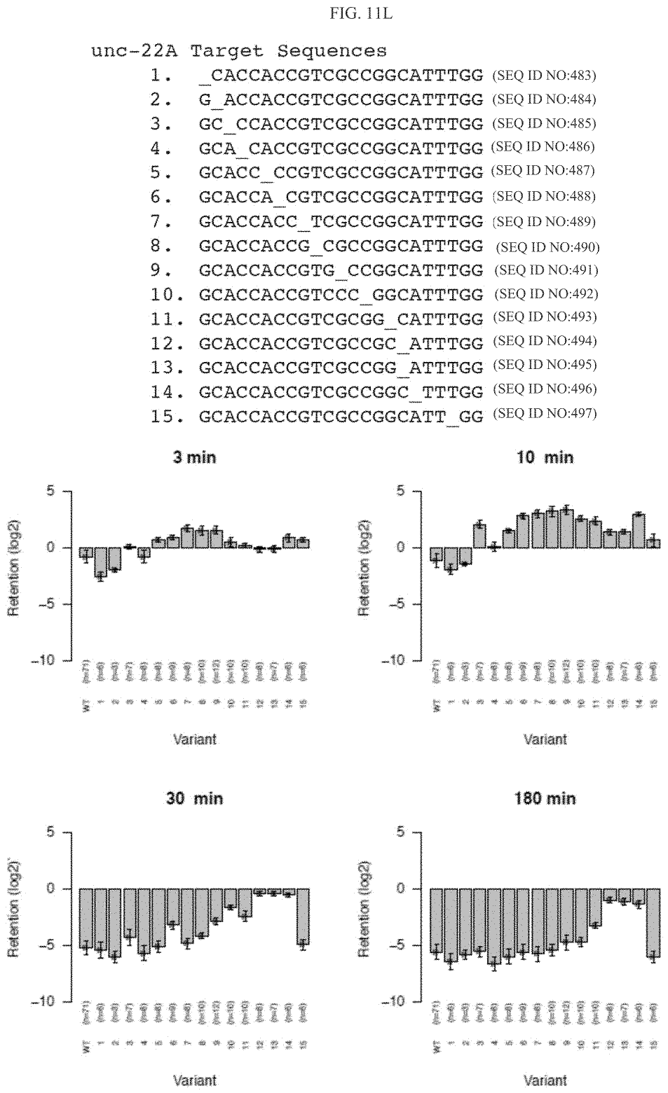

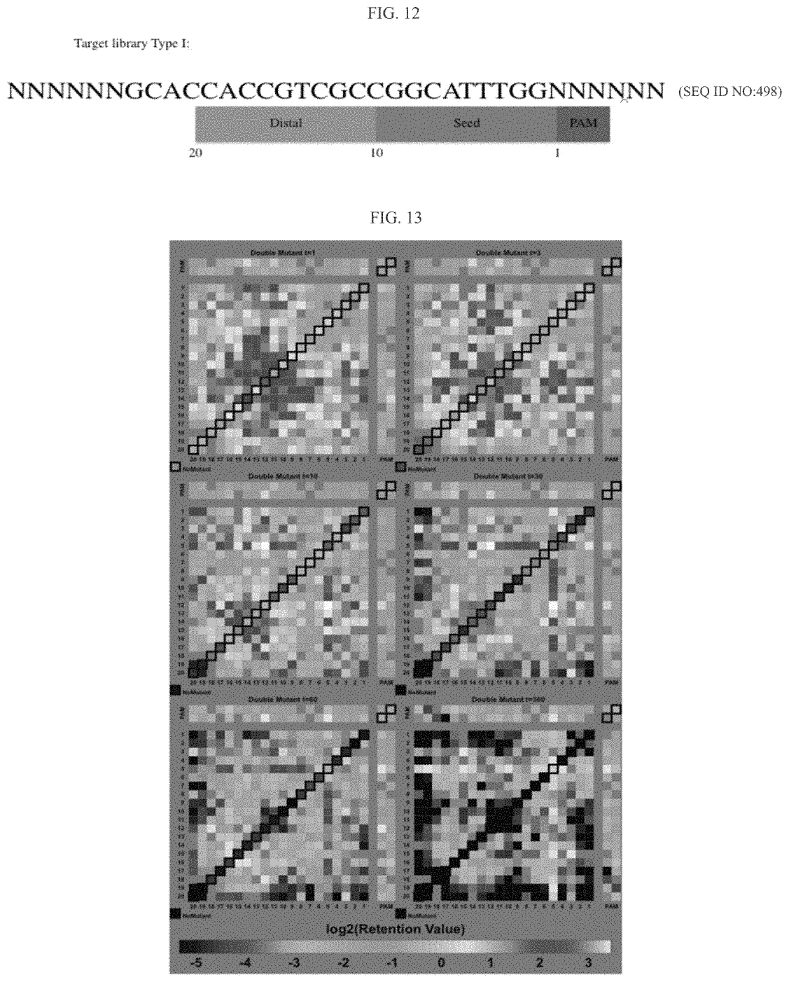

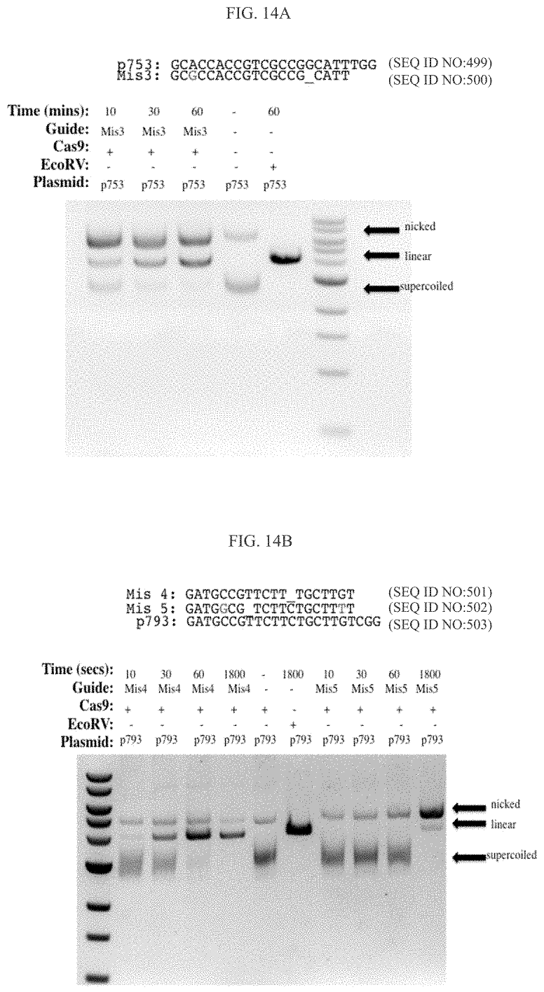

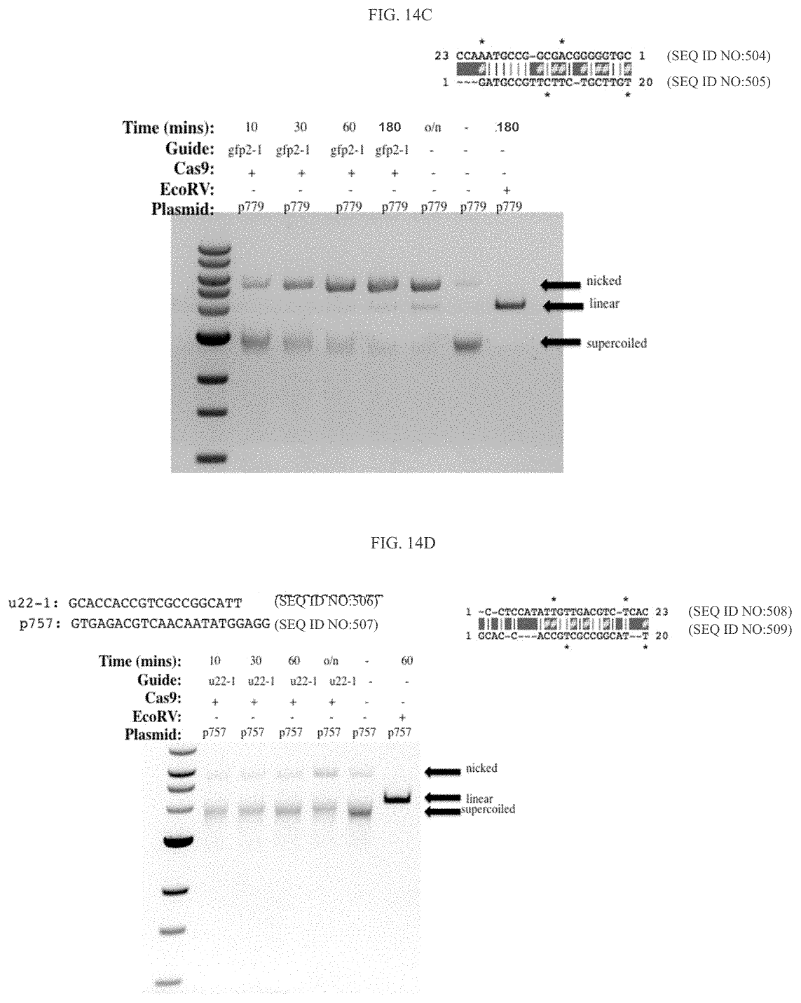

The type II CRISPR effector enzyme Cas9 has been a workhorse tool for genome editing and for a wide variety of experimental applications in vivo and in vitro. S. pyogenes Cas9 was tested for nicking activity in assays and libraries that (as with the tested Cpf1 libraries) contained wild-type, single variants, single deletions, and double consecutive mutations in four targets: EGFP-1, EGFP-2, unc-22A, and rol-6 (FIG. 1B). While yielding some evidence of preferential nicking of specific substrates, these assays yielded a less dramatic distinction between nicking and double strand cleavage for individual template sequences than had been seen with Cpf1 (FIGS. 11A-11L). Encouraged by the differences (but with the knowledge that an optimal nicking activity might require more extensive mutational analysis), the Cas9 assays were repeated using an unc-22A library obtained through random oligonucleotide synthesis with a broader set of multiple mutations (Fu et al.^{19,20}; FIG. 12). These assays yielded a strong preferential nicking for a variety of double mutants (FIG. 13). Examining an extended list of the randomly mutagenized targets from this library for which positive retention scores indicated a strong nicking activity, a design with two mutations, a single deletion in the seed region at position 5 and a mismatch (A to G) in the distal region at position 18 for the unc-22A target was chosen. DNA templates with this double variant reacted with Cas9 complexed to wild-type unc-22A gRNA confirmed robust target specific nicking (FIG. 5A). EGFP-2 variant targets that had a highly positive retention score in the high throughput assays were likewise confirmed as nicking substrates in agarose gel assays (FIG. 5B). Conversely, wild-type unc-22A and EGFP-2 targets were assayed for nicking with the equivalently mismatched (mutant) gRNA. Similar to Cpf1, the equivalent mutations in the gRNA with wild-type targets showed nicking and some linearization that varied depending on gRNAs (FIGS. 14A-14B). Finally, it was observed that some non-intended targets were nicked (e.g., a variant target sequence of unc-22A nicking with wild-type EGFP-2 gRNA). It was hypothesized that targets with certain minimal and/or broken homologies can induce nicking. This was confirmed by testing non-targeting DNA with gRNAs that showed positive retention scores and confirmed the nicking observed in the high throughput results. For example, it was observed in the high throughput assays a variant of the unc-22A target that evidenty nicks with EGFP-2 gRNA (FIG. 14C). This variant brings the unc-22A sequence somewhat closer to the EGFP-2 sequence, although still providing rather limited homology (contiguous complementarity confined to 6 consecutive matches in the seed region). While demonstrating a less stringent sequence requirement, nicking remained a specific process, with only residual nicking for unrelated targets observed in the gel assay (e.g., >16 hour time point; FIG. 14D).

### Example 4. Tuning of Relative Nicking Activity

Analysis of complex libraries at multiple time points or with differing enzyme:substrate ratios provides a combined view that allows selection of substrates with a high preference for nicking over cleavage, with little or no cleavage even on extended incubation. Such substrates are exemplified for Cas9 by unc-22A double transversion (positions 5,12) (FIG. 13). For LbCpf1 maximal nicking is seen at certain time points with double consecutive transversion mutations in positions (12/13 and 14/15; FIGS. 3A-3C, 6A-6F, 7A-7L, and 8A-8I). These examples illustrate the value of an optimization round in identifying the most specific nicking reagents for a given target, and of the value of a broad variant survey in characterizing potential off-target nicking consequences for a given gRNA.

### Example 5. Assessment of Nicking by Cas9 In Vivo

Assessments of Cas9 activity in vivo were carried out in S. cerevisiae, using methodologies of Cas9 and gRNA expression and of target library production described in Fu et al.²⁰. These high throughput assays allow tracking of topology as a function of sequence for a target library, allowing determination of which templates in a complex pool are cut, which are nicked, and which are not cut. While sequencing provides a valuable assay for determining the population of molecules present before and after exposure in vivo to a Cas9+gRNA pair, it remains important to approach such data with considerable care. One concern is that the in vivo situation in yeast will represent a dynamic equilibrium between any cleavage or nicking by the Cas9 enzyme and repair processes (that might fix a nick or break) or other cellular processes (e.g., replication across a nick, generating a break) that might interconvert the various states. To investigate in vivo activities, the incidence of nicked substrates in vivo was evaluated. An alternative approach would have been the much more complicated kinetic question of determining the rate of new nicks in the presence of repair and other activities.

In developing assays to test nicking of DNA in vivo, the simple detection of relaxed circles among extracted DNAs is not sufficient to infer whether (i) nicking had occurred in vivo, or (ii) nicking occurred during extraction and/or analysis of DNA. To ensure definitive resolution of this issue, additional questions needed to be addressed:

1. Is the nicking signal (enhanced PCR yield observed in DNA pools containing close matches to the guide) reproducible in parallel samples and in different biological backgrounds?

2. Is the nicking signal specific to molecules in the target pool with homology to the Cas9 gRNA and PAM site?

3. For partially-matched targets, does the nicking signal decrease for target sequences with substantial numbers of gRNA mismatches?

4. Does the nicking signal increase with induction of Cas9 and with longer exposure times in vivo?

5. Does the in vivo nicking signal for different target variants correlate with measured in vitro nicking?

The analysis applied herein found all the criteria fulfilled, providing support that the state of extracted DNA reflected the configuration in vivo and that a fraction of this DNA was indeed in a nicked form. The nicking signal (preferential retention of gRNA-matched targets in samples processed from uncut yeast DNA) was consistent between replicates of induced yeast (FIG. 16A), depended on both PAM sequence and gRNA homology (FIGS. 16A-16C), was lost with multiple mutations in the target (FIGS. 16B-16C; 4-7 mismatch lane), increased with longer exposure times (FIGS. 16B-16C; comparing single-generation and 2.5 generation samples), and was correlated (R=0.47, p-value=0.00015) with nicking observed in vitro (FIGS. 16D-16F). Based on the observed differential retention (a maximum of ˜3-fold in vivo) and the maximum enhancement for fully nicked DNA (similarly found in Lin et al.¹⁸ and our observations), it was estimated that between ⅙ and ⅓ of susceptible targets were nicked at any time in vivo. A modest difference among in vitro conditions (two different buffers) and the in vivo yeast observations is worth noting. All showed nicking of mismatched (and to some extent, fully matched) targets, while differences were seen in the relative proportions for different sequences. Of interest, a substantial proportion of nicked targets was observed to persist even for fully matched Cas9 targets in the yeast assays (FIGS. 16B-16C, black dots).

### IX. References

1. Terns, M. P. & Terns, R. M. CRISPR-based adaptive immune systems. Curr. Opin. Microbiol. 14, 321-7 (2011).
2. Barrangou, R. et al. CRISPR provides acquired resistance against viruses in prokaryotes. Science 315, 1709-12 (2007).
3. Barrangou, R. & Marraffini, L. A. CRISPR-Cas systems: Prokaryotes upgrade to adaptive immunity. Mol. Cell 54, 234-44 (2014).
4. Heler, R., Marraffini, L. A. & Bikard, D. Adapting to new threats: the generation of memory by CRISPR-Cas immune systems. Mol. Microbiol. 93, 1-9 (2014).
5. Carroll, D. Genome Editing: Past, Present, and Future. Yale J. Biol. Med. 90, 653-659 (2017).
6. Mali, P., Esvelt, K. M. & Church, G. M. Cas9 as a versatile tool for engineering biology. Nat. Methods 10, 957-63 (2013).
7. Terns, R. M. & Terns, M. P. CRISPR-based technologies: prokaryotic defense weapons repurposed. Trends Genet. 30, 111-118 (2014).
8. Jinek, M. et al. A programmable dual-RNA-guided DNA endonuclease in adaptive bacterial immunity. Science 337, 816-21 (2012).
9. Murovec, J., Pirc, Ž. & Yang, B. New variants of CRISPR RNA-guided genome editing enzymes. Plant Biotechnol. J. 15, 917-926 (2017).
10. Cebrian-Serrano, A. & Davies, B. CRISPR-Cas orthologues and variants: optimizing the repertoire, specificity and delivery of genome engineering tools. Mamm. Genome 28, 247-261 (2017).
11. Shen, B. et al. Efficient genome modification by CRISPR-Cas9 nickase with minimal off-target effects. Nat. Methods 11, 399-402 (2014).
12. Davis, L. & Maizels, N. Homology-directed repair of DNA nicks via pathways distinct from canonical double-strand break repair. Proc. Natl. Acad. Sci. U.S.A. 111, E924-32 (2014).
13. Zetsche, B. et al. Cpf1 is a single RNA-guided endonuclease of a class 2 CRISPR-Cas system. Cell 163, 759-71 (2015).
14. Bayat, H., Modarressi, M. H. & Rahimpour, A. The Conspicuity of CRISPR-Cpf1 System as a Significant Breakthrough in Genome Editing. Curr. Microbiol. 75, 107-115 (2018).
15. Fernandes, H., Pastor, M. & Bochtler, M. Type II and type V CRISPR effector nucleases from a structural biologist's perspective. Postepy Biochem. 62, 315-326
16. Deltcheva, E. et al. CRISPR RNA maturation by trans-encoded small RNA and host factor RNase III. Nature 471, 602-7 (2011).
17. Kim, H. K. et al. In vivo high-throughput profiling of CRISPR-Cpf1 activity. Nat. Methods 14, 153-159 (2017).
18. Lin, C.-H., Chen, Y.-C. & Pan, T.-M. Quantification Bias Caused by Plasmid DNA Conformation in Quantitative Real-Time PCR Assay. PLoS One 6, e29101 (2011).
19. Fu, B. X. H., Hansen, L. L., Artiles, K. L., Nonet, M. L. & Fire, A. Z. Landscape of target: guide homology effects on Cas9-mediated cleavage. Nucleic Acids Res. 42, 13778-87 (2014).
20. Fu, B. X. H., St. Onge, R. P., Fire, A. Z. & Smith, J. D. Distinct patterns of Cas9 mismatch tolerance in vitro and in vivo. Nucleic Acids Res. 44, 5365-5377 (2016).
21. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. U.S.A. 109, E2579-86 (2012).
22. Ran, F. A. et al. Double Nicking by RNA-Guided CRISPR Cas9 for Enhanced Genome Editing Specificity. Cell 154, 1380-1389 (2013).
23. Dong, D. et al. The crystal structure of Cpf1 in complex with CRISPR RNA. Nature 532, 522-6 (2016).
24. Fonfara, I., Richter, H., Bratovič, M., Le Rhun, A. & Charpentier, E. The CRISPR-associated DNA-cleaving enzyme Cpf1 also processes precursor CRISPR RNA. Nature 532, 517-21 (2016).
25. Yamano, T. et al. Crystal Structure of Cpf1 in Complex with Guide RNA and Target DNA. Cell 165, 949-962 (2016).
26. Kuzminov, A. Single-strand interruptions in replicating chromosomes cause double-strand breaks. Proc. Natl. Acad. Sci. 98, 8241-8246 (2001).
27. Koo, T., Lee, J. & Kim, J.-S. Measuring and Reducing Off-Target Activities of Programmable Nucleases Including CRISPR-Cas9. Mol. Cells 38, 475-481 (2015).
28. Zhang, X.-H., Tee, L. Y., Wang, X.-G., Huang, Q.-S. & Yang, S.-H. Off-target Effects in CRISPR/Cas9-mediated Genome Engineering. Mol. Ther. Nucleic Acids 4, e264 (2015).
29. Bhaya, D., Davison, M. & Barrangou, R. CRISPR-Cas systems in bacteria and archaea: versatile small RNAs for adaptive defense and regulation. Annu. Rev. Genet. 45, 273-97 (2011).
30. Sternberg, S. H., Richter, H., Charpentier, E. & Qimron, U. Adaptation in CRISPR-Cas Systems. Mol. Cell 61, 797-808 (2016).
31. Jackson, S. A. et al. CRISPR-Cas: Adapting to change. Science (80-.). 356, eaa15056 (2017).
32. Wei, Y., Terns, R. M. & Terns, M. P. Cas9 function and host genome sampling in Type II-A CRISPR-Cas adaptation. Genes Dev. 29, 356-61 (2015).
33. Heler, R. et al. Cas9 specifies functional viral targets during CRISPR-Cas adaptation. Nature 519, 199-202 (2015).
34. Fineran, P. C. et al. Degenerate target sites mediate rapid primed CRISPR adaptation. Proc. Natl. Acad. Sci. U.S.A. 111, E1629-38 (2014).
35. Staals, R. H. J. et al. Interference-driven spacer acquisition is dominant over naive and primed adaptation in a native CRISPR-Cas system. Nat. Commun. 7, 12853 (2016).
36. Richter, C. et al. Priming in the Type I-F CRISPR-Cas system triggers strand-independent spacer acquisition, bi-directionally from the primed protospacer. Nucleic Acids Res. 42, 8516-26 (2014).
37. Gao, Y. et al. Single Cas9 nickase induced generation of NRAMP1 knockin cattle with reduced off-target effects. Genome Biol. 18, 13 (2017).
38. Satomura, A. et al. Precise genome-wide base editing by the CRISPR Nickase system in yeast. Sci. Rep. 7, 2095 (2017).
39. Chen, J. S. et al. CRISPR-Cas12a target binding unleashes indiscriminate single-stranded DNase activity. Science 360(6387), 436-439 (2018).

### X. Exemplary Embodiments

Exemplary embodiments provided in accordance with the presently disclosed subject matter include, but are not limited to, the claims and the following embodiments:

1. A dual nickase CRISPR system comprising:

(a) a first CRISPR-Cas nickase comprising a first CRISPR-Cas nuclease and a first guide RNA (gRNA) comprising a first targeting region capable of guiding the first nuclease to cleave one strand of a double-stranded target DNA sequence, wherein the first gRNA comprises at least one nucleotide mismatch or deletion in the first targeting region relative to the target DNA sequence; and
(b) a second CRISPR-Cas nickase comprising a second CRISPR-Cas nuclease and a second gRNA comprising a second targeting region capable of guiding the second nuclease to cleave the complementary strand of the target DNA sequence, wherein the second gRNA comprises at least one nucleotide mismatch or deletion in the second targeting region relative to the target DNA sequence.

2. The dual nickase CRISPR system of embodiment 1, wherein the first CRISPR-Cas nuclease and/or the second CRISPR-Cas nuclease is a wild-type CRISPR-Cas nuclease.

3. The dual nickase CRISPR system of embodiment 1 or 2, wherein the first CRISPR-Cas nuclease and/or the second CRISPR-Cas nuclease is a mutant CRISPR-Cas nuclease.

4. A composition comprising a first RNA-guided DNA nuclease, a first guide RNA (gRNA), a second RNA-guided DNA nuclease, and a second gRNA,

wherein the first gRNA comprises at least one nucleotide mismatch or deletion relative to a region of a first strand of a target DNA,
wherein the second gRNA comprises at least one nucleotide mismatch or deletion relative to a region of a second strand of the target DNA,
wherein the first gRNA guides the first nuclease to bind to and cleave in the region of the first strand of the target DNA,
wherein the second gRNA guides the second nuclease to bind to and cleave in the region of the second strand of the target DNA, and
wherein cleavage of the first and second strands of the target DNA by the first and second nucleases produces a double-strand break in the target DNA.

5. The composition of embodiment 4, wherein the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA.

6. The composition of embodiment 5, wherein the at least one nucleotide mismatch relative to the region of the first strand of the target DNA is a transversion point mutation and/or wherein the at least one nucleotide mismatch relative to the region of the second strand of the target DNA is a transversion point mutation.

7. The composition of embodiment 5 or 6, wherein the first gRNA comprises a nucleotide deletion in a seed region and a nucleotide mismatch in a distal region of the of the first gRNA relative to the region of the first strand of the target DNA, and wherein the second gRNA comprises a nucleotide deletion in a seed region and a nucleotide mismatch in a distal region of the second gRNA relative to the region of the second strand of the target DNA.

8. The composition of embodiment 5 or 6, wherein the first gRNA comprises a nucleotide mismatch that is a transversion point mutation in a seed region and a nucleotide mismatch that is a transversion point mutation in a distal region of the of the first gRNA relative to the region of the first strand of the target DNA, and wherein the second gRNA comprises two nucleotide mismatches that are transversion point mutations in a distal region of the second gRNA relative to the region of the second strand of the target DNA.

9. The composition of any one of embodiments 4 to 8, wherein the first RNA-guided DNA nuclease and/or the second RNA-guided DNA nuclease is a wild-type CRISPR-Cas nuclease.

10. The composition of any one of embodiments 4 to 9, wherein the first RNA-guided DNA nuclease and/or the second RNA-guided DNA nuclease is a mutant CRISPR-Cas nuclease.

11. A nickase CRISPR system comprising: a CRISPR-Cas nickase comprising a CRISPR-Cas nuclease and a guide RNA (gRNA) comprising a targeting region capable of guiding the nuclease to cleave one strand of a double-stranded target DNA sequence, wherein the gRNA comprises at least one nucleotide mismatch or deletion in the targeting region relative to the target DNA sequence.

12. The nickase CRISPR system of embodiment 11, wherein the CRISPR-Cas nuclease is a wild-type CRISPR-Cas nuclease.

13. The nickase CRISPR system of embodiment 11, wherein the CRISPR-Cas nuclease is a mutant CRISPR-Cas nuclease.

14. A composition comprising a RNA-guided DNA nuclease and a guide RNA (gRNA), wherein the gRNA comprises at least one nucleotide mismatch or deletion relative to a region in a target DNA, wherein the gRNA guides the nuclease to bind to and cleave in the region in the target DNA, and wherein cleavage of the target DNA by the nuclease produces a single-strand break in the target DNA.

15. The composition of embodiment 14, wherein the at least one nucleotide mismatch or deletion relative to the region in the target DNA is in a distal region of the gRNA.

16. The composition of embodiment 15, wherein the at least one nucleotide mismatch relative to the region in the target DNA is a transversion point mutation.

17. The composition of embodiment 15 or 16, wherein the gRNA comprises a nucleotide deletion in a seed region and a nucleotide mismatch in a distal region of the of the gRNA relative to the region in the target DNA.

18. The composition of embodiment 15 or 16, wherein the gRNA comprises a nucleotide mismatch that is a transversion point mutation in a seed region and a nucleotide mismatch that is a transversion point mutation in a distal region of the of the gRNA relative to the region in the target DNA.

19. The composition of any one of embodiments 14 to 18, wherein the RNA-guided DNA nuclease is a wild-type CRISPR-Cas nuclease.

20. The composition of any one of embodiments 14 to 18, wherein the RNA-guided DNA nuclease is a mutant CRISPR-Cas nuclease.

21. A method of cleaving both strands of a target DNA, comprising providing a first RNA-guided DNA nickase, a first gRNA, a second RNA-guided DNA nickase, and a second gRNA,

wherein the first gRNA comprises at least one nucleotide mismatch to a region of a first strand in the target DNA,
the second gRNA comprises at least one nucleotide mismatch to a region of a second strand in the target DNA,
the first gRNA guides the first RNA-guided DNA nickase to bind to and cleave in the region of the first strand of the target DNA,
the second gRNA guides the second RNA-guided DNA nickase to bind to and cleave in the region of the second strand of the target DNA, and
cleavage of both strands by the first and second RNA-guided DNA nickases produces two product DNAs each having an overhang of at least one nucleotide.

22. The method of embodiment 21, wherein the first RNA-guided DNA nickase and/or the second RNA-guided DNA nickase is a wild-type RNA-guided DNA nickase.

23. The method of embodiment 21 or 22, wherein the first RNA-guided DNA nickase and/or the second RNA-guided DNA nickase is a mutant RNA-guided DNA nickase.

24. The method of any one of embodiments 21 to 23, wherein the method comprises in vivo cleavage of both strands of the target DNA.

25. A method of cleaving both strands of a target DNA, comprising providing an RNA-guided DNA nickase, a first gRNA, and a second gRNA,

wherein the first gRNA comprises at least one nucleotide mismatch to a region of a first strand in the target DNA,
the second gRNA comprises at least one nucleotide mismatch to a region of a second strand in the target DNA,
the first gRNA guides the RNA-guided DNA nickase to bind to and cleave in the region of the first strand of the target DNA,
the second gRNA guides the RNA-guided DNA nickase to bind to and cleave in the region of the second strand of the target DNA, and
cleavage of both strands by the RNA-guided DNA nickase produces two product DNAs each having an overhang of at least one nucleotide.

26. The method of embodiment 25, wherein the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA.

27. The method of embodiment 26, wherein the at least one nucleotide mismatch relative to the region of the first strand of the target DNA is a transversion point mutation and/or wherein the at least one nucleotide mismatch relative to the region of the second strand of the target DNA is a transversion point mutation.

28. The method of embodiment 26 or 27, wherein the first gRNA comprises a nucleotide deletion in a seed region and a nucleotide mismatch in a distal region of the of the first gRNA relative to the region of the first strand of the target DNA, and wherein the second gRNA comprises a nucleotide deletion in a seed region and a nucleotide mismatch in a distal region of the second gRNA relative to the region of the second strand of the target DNA.

29. The method of embodiment 26 or 27, wherein the first gRNA comprises a nucleotide mismatch that is a transversion point mutation in a seed region and a nucleotide mismatch that is a transversion point mutation in a distal region of the of the first gRNA relative to the region of the first strand of the target DNA, and wherein the second gRNA comprises two nucleotide mismatches that are transversion point mutations in a distal region of the second gRNA relative to the region of the second strand of the target DNA.

30. The method of any one of embodiments 25 to 29, wherein the RNA-guided DNA nickase is a wild-type RNA-guided DNA nickase.

31. The method of any one of embodiments 25 to 29, wherein the RNA-guided DNA nickase is a mutant RNA-guided DNA nickase.

32. The method of any one of embodiments 25 to 31, wherein the method comprises in vivo cleavage of both strands of the target DNA.

33. A method of directing an RNA-guided DNA nickase to a region in a target DNA, comprising contacting the target DNA with the RNA-guided DNA nickase and a gRNA, wherein the gRNA comprises at least one nucleotide mismatch to the region in the target DNA and the gRNA guides the RNA-guided DNA nickase to bind to and cleave in the region in the target DNA.

34. The method of embodiment 33, wherein the RNA-guided DNA nickase is immobilized on the target DNA.

35. The method of embodiment 33 or 34, wherein the target DNA is contacted with the RNA-guided DNA nickase and the gRNA in vivo.

What is claimed is:

1. A dual nickase CRISPR system comprising:
(a) a first CRISPR-Cas nickase comprising a first CRISPR-Cas nuclease and a first guide RNA (gRNA) comprising a first targeting region capable of guiding the first nuclease to cleave one strand of a double-stranded target DNA sequence, wherein the first gRNA comprises at least one nucleotide mismatch or deletion in the first targeting region relative to the target DNA sequence; and
(b) a second CRISPR-Cas nickase comprising a second CRISPR-Cas nuclease and a second gRNA comprising a second targeting region capable of guiding the second nuclease to cleave the complementary strand of the target DNA sequence, wherein the second gRNA comprises at least one nucleotide mismatch or deletion in the second targeting region relative to the target DNA sequence.

2. The dual nickase CRISPR system of claim 1, wherein the first CRISPR-Cas nuclease and/or the second CRISPR-Cas nuclease is a wild-type CRISPR-Cas nuclease.

3. The dual nickase CRISPR system of claim 1, wherein the first CRISPR-Cas nuclease and/or the second CRISPR-Cas nuclease is a mutant CRISPR-Cas nuclease.

4. A composition comprising a first RNA-guided DNA nuclease, a first guide RNA (gRNA), a second RNA-guided DNA nuclease, and a second gRNA,
wherein the first gRNA comprises at least one nucleotide mismatch or deletion relative to a region of a first strand of a target DNA,
wherein the second gRNA comprises at least one nucleotide mismatch or deletion relative to a region of a second strand of the target DNA,
wherein the first gRNA guides the first nuclease to bind to and cleave in the region of the first strand of the target DNA,
wherein the second gRNA guides the second nuclease to bind to and cleave in the region of the second strand of the target DNA, and
wherein cleavage of the first and second strands of the target DNA by the first and second nucleases produces a double-strand break in the target DNA.

5. The composition of claim 4, wherein the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA.

6. The composition of claim 5, wherein the at least one nucleotide mismatch relative to the region of the first strand of the target DNA is a transversion point mutation and/or wherein the at least one nucleotide mismatch relative to the region of the second strand of the target DNA is a transversion point mutation.

7. The composition of claim 5, wherein the first gRNA comprises a nucleotide deletion in a seed region and a nucleotide mismatch in a distal region of the of the first gRNA relative to the region of the first strand of the target DNA, and
wherein the second gRNA comprises a nucleotide deletion in a seed region and a nucleotide mismatch in a distal region of the second gRNA relative to the region of the second strand of the target DNA.

8. The composition of claim 5, wherein the first gRNA comprises a nucleotide mismatch that is a transversion point mutation in a seed region and a nucleotide mismatch that is a transversion point mutation in a distal region of the of the first gRNA relative to the region of the first strand of the target DNA, and
wherein the second gRNA comprises two nucleotide mismatches that are transversion point mutations in a distal region of the second gRNA relative to the region of the second strand of the target DNA.

9. The composition of claim 4, wherein the first RNA-guided DNA nuclease and/or the second RNA-guided DNA nuclease is a wild-type CRISPR-Cas nuclease.

10. The composition of claim 4, wherein the first RNA-guided DNA nuclease and/or the second RNA-guided DNA nuclease is a mutant CRISPR-Cas nuclease.

11. A nickase CRISPR system comprising: a CRISPR-Cas nickase comprising a CRISPR-Cas nuclease and a guide RNA (gRNA) comprising a targeting region capable of guiding the nuclease to cleave one strand of a double-stranded target DNA sequence, wherein the gRNA comprises at least one nucleotide mismatch or deletion in the targeting region relative to the target DNA sequence.

12. The nickase CRISPR system of claim 11, wherein the CRISPR-Cas nuclease is a wild-type CRISPR-Cas nuclease.

13. The nickase CRISPR system of claim 11, wherein the CRISPR-Cas nuclease is a mutant CRISPR-Cas nuclease.

14. A composition comprising a RNA-guided DNA nuclease and a guide RNA (gRNA), wherein the gRNA comprises at least one nucleotide mismatch or deletion relative to a region in a target DNA, wherein the gRNA guides the nuclease to bind to and cleave in the region in the target DNA, and wherein cleavage of the target DNA by the nuclease produces a single-strand break in the target DNA.

15. The composition of claim 14, wherein the at least one nucleotide mismatch or deletion relative to the region in the target DNA is in a distal region of the gRNA.

16. The composition of claim 15, wherein the at least one nucleotide mismatch relative to the region in the target DNA is a transversion point mutation.

17. The composition of claim 15, wherein the gRNA comprises a nucleotide deletion in a seed region and a nucleotide mismatch in a distal region of the of the gRNA relative to the region in the target DNA.

18. The composition of claim 15, wherein the gRNA comprises a nucleotide mismatch that is a transversion point mutation in a seed region and a nucleotide mismatch that is a transversion point mutation in a distal region of the of the gRNA relative to the region in the target DNA.

19. The composition of claim 14, wherein the RNA-guided DNA nuclease is a wild-type CRISPR-Cas nuclease.

20. The composition of claim 14, wherein the RNA-guided DNA nuclease is a mutant CRISPR-Cas nuclease.

21. A method of cleaving both strands of a target DNA, comprising providing a first RNA-guided DNA nickase, a first gRNA, a second RNA-guided DNA nickase, and a second gRNA,
wherein the first gRNA comprises at least one nucleotide mismatch to a region of a first strand in the target DNA,
the second gRNA comprises at least one nucleotide mismatch to a region of a second strand in the target DNA,
the first gRNA guides the first RNA-guided DNA nickase to bind to and cleave in the region of the first strand of the target DNA,
the second gRNA guides the second RNA-guided DNA nickase to bind to and cleave in the region of the second strand of the target DNA, and
cleavage of both strands by the first and second RNA-guided DNA nickases produces two product DNAs each having an overhang of at least one nucleotide.

22. The method of claim 21, wherein the first RNA-guided DNA nickase and/or the second RNA-guided DNA nickase is a wild-type RNA-guided DNA nickase.

23. The method of claim 21, wherein the first RNA-guided DNA nickase and/or the second RNA-guided DNA nickase is a mutant RNA-guided DNA nickase.

24. The method of claim 21, wherein the method comprises in vivo cleavage of both strands of the target DNA.

25. A method of cleaving both strands of a target DNA, comprising providing an RNA-guided DNA nickase, a first gRNA, and a second gRNA,
wherein the first gRNA comprises at least one nucleotide mismatch to a region of a first strand in the target DNA,
the second gRNA comprises at least one nucleotide mismatch to a region of a second strand in the target DNA,
the first gRNA guides the RNA-guided DNA nickase to bind to and cleave in the region of the first strand of the target DNA,
the second gRNA guides the RNA-guided DNA nickase to bind to and cleave in the region of the second strand of the target DNA, and
cleavage of both strands by the RNA-guided DNA nickase produces two product DNAs each having an overhang of at least one nucleotide.

26. The method of claim 25, wherein the at least one nucleotide mismatch or deletion relative to the region of the first strand of the target DNA is in a distal region of the first gRNA, and wherein the at least one nucleotide mismatch or deletion relative to the region of the second strand of the target DNA is in a distal region of the second gRNA.

27. The method of claim 26, wherein the at least one nucleotide mismatch relative to the region of the first strand of the target DNA is a transversion point mutation and/or wherein the at least one nucleotide mismatch relative to the region of the second strand of the target DNA is a transversion point mutation.

28. The method of claim 26, wherein the first gRNA comprises a nucleotide deletion in a seed region and a nucleotide mismatch in a distal region of the of the first gRNA relative to the region of the first strand of the target DNA, and
wherein the second gRNA comprises a nucleotide deletion in a seed region and a nucleotide mismatch in a distal region of the second gRNA relative to the region of the second strand of the target DNA.

29. The method of claim 26, wherein the first gRNA comprises a nucleotide mismatch that is a transversion point mutation in a seed region and a nucleotide mismatch that is a transversion point mutation in a distal region of the of the first gRNA relative to the region of the first strand of the target DNA, and
wherein the second gRNA comprises two nucleotide mismatches that are transversion point mutations in a distal region of the second gRNA relative to the region of the second strand of the target DNA.

30. The method of claim 25, wherein the RNA-guided DNA nickase is a wild-type RNA-guided DNA nickase.

31. The method of claim 25, wherein the RNA-guided DNA nickase is a mutant RNA-guided DNA nickase.

32. The method of claim 25, wherein the method comprises in vivo cleavage of both strands of the target DNA.

33. A method of directing an RNA-guided DNA nickase to a region in a target DNA, comprising contacting the target DNA with the RNA-guided DNA nickase and a gRNA, wherein the gRNA comprises at least one nucleotide mismatch to the region in the target DNA and the gRNA guides the RNA-guided DNA nickase to bind to and cleave in the region in the target DNA.

34. The method of claim 33, wherein the RNA-guided DNA nickase is immobilized on the target DNA.

35. The method of claim 33, wherein the target DNA is contacted with the RNA-guided DNA nickase and the gRNA in vivo.


SEQUENCE LISTING (534 sequences)

SEQ ID NO: 1 - DNA, 36 bp, Artificial Sequence
  Description of Artificial Sequence Synthetic oligonucleotide
  modified_base (32)..(36): a, c, t, g, unknown or other
tgctttaccggcaagctgcccgtgccctggcnnnnn

SEQ ID NO: 2 - DNA, 36 bp, Artificial Sequence
  Description of Artificial Sequence Synthetic oligonucleotide
  modified_base (32)..(36): a, c, t, g, unknown or other
atgtttgacaagcagaagaacggcatcaaggnnnnn

SEQ ID NO: 3 - DNA, 36 bp, Artificial Sequence
  Description of Artificial Sequence Synthetic oligonucleotide
  modified_base (32)..(36): a, c, t, g, unknown or other
atattttccatattgttgacgtctcacacggnnnnn

SEQ ID NO: 4 - DNA, 36 bp, Artificial Sequence
  Description of Artificial Sequence Synthetic oligonucleotide
  modified_base (32)..(36): a, c, t, g, unknown or other
ggatttaaatgccggcgacggtggtgcctacnnnnn

SEQ ID NO: 5 - DNA, 36 bp, Artificial Sequence
  Description of Artificial Sequence Synthetic oligonucleotide
  modified_base (32)..(36): a, c, t, g, unknown or other
cgcttttcatggaatcccttctgcagcacctnnnnn

SEQ ID NO: 6 - DNA, 36 bp, Artificial Sequence
  Description of Artificial Sequence Synthetic oligonucleotide
  modified_base (32)..(36): a, c, t, g, unknown or other
gccagggcacgggcagcttgccggtggtgcannnnn

SEQ ID NO: 7 - DNA, 36 bp, Artificial Sequence
  Description of Artificial Sequence Synthetic oligonucleotide
  modified_base (32)..(36): a, c, t, g, unknown or other
ccttgatgccgttcttctgcttgtcggccatnnnnn

SEQ ID NO: 8 - DNA, 36 bp, Artificial Sequence
  Description of Artificial Sequence Synthetic oligonucleotide
  modified_base (32)..(36): a, c, t, g, unknown or other
ccgtgtgagacgtcaacaatatggaggatatnnnnn

SEQ ID NO: 9 - DNA, 36 bp, Artificial Sequence
  Description of Artificial Sequence Synthetic oligonucleotide
  modified_base (32)..(36): a, c, t, g, unknown or other
gtaggcaccaccgtcgccggcatttggttccnnnnn

SEQ ID NO: 10 - DNA, 23 bp, Artificial Sequence
  Description of Artificial Sequence Synthetic oligonucleotide
tttacggcaagctgcccgtgccc

SEQ ID NO: 11 - DNA, 23 bp, Artificial Sequence
  Description of Artificial Sequence Synthetic oligonucleotide
tttaccgcaagctgcccgtgccc

SEQ ID NO: 12 - DNA, 23 bp, Artificial Sequence
  Description of Artificial Sequence Synthetic oligonucleotide
tttaccggaagctgcccgtgccc

SEQ ID NO: 13 - DNA, 23 bp, Artificial Sequence
  Description of Artificial Sequence Synthetic oligonucleotide
tttaccggcagctgcccgtgccc

SEQ ID NO: 14 - DNA, 23 bp, Artificial Sequence
  Description of Artificial Sequence Synthetic oligonucleotide
tttaccggcaactgcccgtgccc

SEQ ID NO: 15 - DNA, 23 bp, Artificial Sequence
  Description of Artificial Sequence Synthetic oligonucleotide
tttaccggcaagtgcccgtgccc

SEQ ID NO: 16 - DNA, 23 bp, Artificial Sequence
  Description of Artificial Sequence Synthetic oligonucleotide
tttaccggcaagcgcccgtgccc

SEQ ID NO: 17 - DNA, 23 bp, Artificial Sequence
  Description of Artificial Sequence Synthetic oligonucleotide
tttaccggcaagctcccgtgccc

SEQ ID NO: 18 - DNA, 23 bp, Artificial Sequence
  Description of Artificial Sequence Synthetic oligonucleotide
tttaccggcaagctgccgtgccc

SEQ ID NO: 19 - DNA, 23 bp, Artificial Sequence
  Description of Artificial Sequence Synthetic oligonucleotide
tttaccggcaagctgccctgccc

SEQ ID NO: 20 - DNA, 23 bp, Artificial Sequence
  Description of Artificial Sequence Synthetic oligonucleotide
tttaccggcaagctgcccggccc

SEQ ID NO: 21 - DNA, 23 bp, Artificial Sequence
  Description of Artificial Sequence Synthetic oligonucleotide
tttaccggcaagctgcccgtccc

SEQ ID NO: 22 - DNA, 24 bp, Artificial Sequence
  Description of Artificial Sequence Synthetic oligonucleotide
tggaccggcaagctgcccgtgccc

SEQ ID NO: 23 - DNA, 24 bp, Artificial Sequence
  Description of Artificial Sequence Synthetic oligonucleotide
ttgcccggcaagctgcccgtgccc

SEQ ID NO: 24 - DNA, 24 bp, Artificial Sequence
  Description of Artificial Sequence Synthetic oligonucleotide
tttcacggcaagctgcccgtgccc

SEQ ID NO: 25 - DNA, 24 bp, Artificial Sequence
  Description of Artificial Sequence Synthetic oligonucleotide
tttaaaggcaagctgcccgtgccc

SEQ ID NO: 26 - DNA, 24 bp, Artificial Sequence
  Description of Artificial Sequence Synthetic oligonucleotide
tttacatgcaagctgcccgtgccc

SEQ ID NO: 27 - DNA, 24 bp, Artificial Sequence
  Description of Artificial Sequence Synthetic oligonucleotide
tttaccttcaagctgcccgtgccc

SEQ ID NO: 28 - DNA, 24 bp, Artificial Sequence
  Description of Artificial Sequence Synthetic oligonucleotide
tttaccgtaaagctgcccgtgccc

SEQ ID NO: 29 - DNA, 24 bp, Artificial Sequence
  Description of Artificial Sequence Synthetic oligonucleotide
tttaccggacagctgcccgtgccc

SEQ ID NO: 30 - DNA, 24 bp, Artificial Sequence
  Description of Artificial Sequence Synthetic oligonucleotide
tttaccggcccgctgcccgtgccc

SEQ ID NO: 31 - DNA, 24 bp, Artificial Sequence
  Description of Artificial Sequence Synthetic oligonucleotide
tttaccggcactctgcccgtgccc

SEQ ID NO: 32 - DNA, 24 bp, Artificial Sequence
  Description of Artificial Sequence Synthetic oligonucleotide
tttaccggcaatatgcccgtgccc

SEQ ID NO: 33 - DNA, 24 bp, Artificial Sequence
  Description of Artificial Sequence Synthetic oligonucleotide
tttaccggcaagaggcccgtgccc

SEQ ID NO: 34 - DNA, 24 bp, Artificial Sequence
  Description of Artificial Sequence Synthetic oligonucleotide
tttaccggcaagcgtcccgtgccc

SEQ ID NO: 35 - DNA, 24 bp, Artificial Sequence
  Description of Artificial Sequence Synthetic oligonucleotide
tttaccggcaagcttaccgtgccc

SEQ ID NO: 36 - DNA, 24 bp, Artificial Sequence
  Description of Artificial Sequence Synthetic oligonucleotide
tttaccggcaagctgaacgtgccc

SEQ ID NO: 37 - DNA, 24 bp, Artificial Sequence
  Description of Artificial Sequence Synthetic oligonucleotide
tttaccggcaagctgcaagtgccc

SEQ ID NO: 38 - DNA, 24 bp, Artificial Sequence
  Description of Artificial Sequence Synthetic oligonucleotide
tttaccggcaagctgccattgccc

SEQ ID NO: 39 - DNA, 24 bp, Artificial Sequence
  Description of Artificial Sequence Synthetic oligonucleotide
tttaccggcaagctgccctggccc

SEQ ID NO: 40 - DNA, 24 bp, Artificial Sequence
  Description of Artificial Sequence Synthetic oligonucleotide
tttaccggcaagctgcccggtccc

SEQ ID NO: 41 - DNA, 23 bp, Artificial Sequence
  Description of Artificial Sequence Synthetic oligonucleotide
tttacggcaagctgcccgtgccc

SEQ ID NO: 42 - DNA, 23 bp, Artificial Sequence
  Description of Artificial Sequence Synthetic oligonucleotide
tttaccgcaagctgcccgtgccc

SEQ ID NO: 43 - DNA, 23 bp, Artificial Sequence
  Description of Artificial Sequence Synthetic oligonucleotide
tttaccggaagctgcccgtgccc

SEQ ID NO: 44 - DNA, 23 bp, Artificial Sequence
  Description of Artificial Sequence Synthetic oligonucleotide
tttaccggcagctgcccgtgccc

SEQ ID NO: 45 - DNA, 23 bp, Artificial Sequence
  Description of Artificial Sequence Synthetic oligonucleotide
tttaccggcaactgcccgtgccc

SEQ ID NO: 46 - DNA, 23 bp, Artificial Sequence
  Description of Artificial Sequence Synthetic oligonucleotide
tttaccggcaagtgcccgtgccc

SEQ ID NO: 47 - DNA, 23 bp, Artificial Sequence
  Description of Artificial Sequence Synthetic oligonucleotide
tttaccggcaagcgcccgtgccc

SEQ ID NO: 48 - DNA, 23 bp, Artificial Sequence
  Description of Artificial Sequence Synthetic oligonucleotide
tttaccggcaagctcccgtgccc

SEQ ID NO: 49 - DNA, 23 bp, Artificial Sequence
  Description of Artificial Sequence Synthetic oligonucleotide
tttaccggcaagctgccgtgccc

SEQ ID NO: 50 - DNA, 23 bp, Artificial Sequence
  Description of Artificial Sequence Synthetic oligonucleotide
tttaccggcaagctgccctgccc

SEQ ID NO: 51 - DNA, 23 bp, Artificial Sequence
  Description of Artificial Sequence Synthetic oligonucleotide
tttaccggcaagctgcccggccc

SEQ ID NO: 52 - DNA, 23 bp, Artificial Sequence
  Description of Artificial Sequence Synthetic oligonucleotide
tttaccggcaagctgcccgtccc

SEQ ID NO: 53 - DNA, 24 bp, Artificial Sequence
  Description of Artificial Sequence Synthetic oligonucleotide
tggaccggcaagctgcccgtgccc

SEQ ID NO: 54 - DNA, 24 bp, Artificial Sequence
  Description of Artificial Sequence Synthetic oligonucleotide
ttgcccggcaagctgcccgtgccc

SEQ ID NO: 55 - DNA, 24 bp, Artificial Sequence
  Description of Artificial Sequence Synthetic oligonucleotide
tttcacggcaagctgcccgtgccc

SEQ ID NO: 56 - DNA, 24 bp, Artificial Sequence
  Description of Artificial Sequence Synthetic oligonucleotide
tttaaaggcaagctgcccgtgccc

SEQ ID NO: 57 - DNA, 24 bp, Artificial Sequence
  Description of Artificial Sequence Synthetic oligonucleotide
tttacatgcaagctgcccgtgccc

SEQ ID NO: 58 - DNA, 24 bp, Artificial Sequence
  Description of Artificial Sequence Synthetic oligonucleotide
tttaccttcaagctgcccgtgccc

SEQ ID NO: 59 - DNA, 24 bp, Artificial Sequence
  Description of Artificial Sequence Synthetic oligonucleotide
tttaccgtaaagctgcccgtgccc

SEQ ID NO: 60 - DNA, 24 bp, Artificial Sequence
  Description of Artificial Sequence Synthetic oligonucleotide
tttaccggacagctgcccgtgccc

SEQ ID NO: 61 - DNA, 24 bp, Artificial Sequence
  Description of Artificial Sequence Synthetic oligonucleotide
tttaccggcccgctgcccgtgccc

SEQ ID NO: 62 - DNA, 24 bp, Artificial Sequence
  Description of Artificial Sequence Synthetic oligonucleotide
tttaccggcactctgcccgtgccc

SEQ ID NO: 63 - DNA, 24 bp, Artificial Sequence
  Description of Artificial Sequence Synthetic oligonucleotide
tttaccggcaatatgcccgtgccc

SEQ ID NO: 64 - DNA, 24 bp, Artificial Sequence
  Description of Artificial Sequence Synthetic oligonucleotide
tttaccggcaagaggcccgtgccc

SEQ ID NO: 65 - DNA, 24 bp, Artificial Sequence
  Description of Artificial Sequence Synthetic oligonucleotide
tttaccggcaagcgtcccgtgccc

SEQ ID NO: 66 - DNA, 24 bp, Artificial Sequence
  Description of Artificial Sequence Synthetic oligonucleotide
tttaccggcaagcttaccgtgccc

SEQ ID NO: 67 - DNA, 24 bp, Artificial Sequence
  Description of Artificial Sequence Synthetic oligonucleotide
tttaccggcaagctgaacgtgccc

SEQ ID NO: 68 - DNA, 24 bp, Artificial Sequence
  Description of Artificial Sequence Synthetic oligonucleotide
tttaccggcaagctgcaagtgccc

SEQ ID NO: 69 - DNA, 24 bp, Artificial Sequence
  Description of Artificial Sequence Synthetic oligonucleotide
tttaccggcaagctgccattgccc

SEQ ID NO: 70 - DNA, 24 bp, Artificial Sequence
  Description of Artificial Sequence Synthetic oligonucleotide
tttaccggcaagctgccctggccc

SEQ ID NO: 71 - DNA, 24 bp, Artificial Sequence
  Description of Artificial Sequence Synthetic oligonucleotide
tttaccggcaagctgcccggtccc

SEQ ID NO: 72 - DNA, 24 bp, Artificial Sequence
  Description of Artificial Sequence Synthetic oligonucleotide
tttaccggcaagctgccattgccc

SEQ ID NO: 73 - DNA, 24 bp, Artificial Sequence
  Description of Artificial Sequence Synthetic oligonucleotide
tttaccggcaagctgcccgtgccc

SEQ ID NO: 74 - DNA, 20 bp, Artificial Sequence
  Description of Artificial Sequence Synthetic oligonucleotide
ccggcaagctgcccgtgccc

SEQ ID NO: 75 - DNA, 24 bp, Artificial Sequence
  Description of Artificial Sequence Synthetic oligonucleotide
tttaccggcaagctgaacgtgccc

SEQ ID NO: 76 - DNA, 20 bp, Artificial Sequence
  Description of Artificial Sequence Synthetic oligonucleotide
ccggcaagctgcccgtgccc

SEQ ID NO: 77 - DNA, 20 bp, Artificial Sequence
  Description of Artificial Sequence Synthetic oligonucleotide
ccggcaagctgccattgccc

SEQ ID NO: 78 - DNA, 24 bp, Artificial Sequence
  Description of Artificial Sequence Synthetic oligonucleotide
tttaccggcaagctgcccgtgccc

SEQ ID NO: 79 - DNA, 20 bp, Artificial Sequence
  Description of Artificial Sequence Synthetic oligonucleotide
aatgccggcgacggtggtgc

SEQ ID NO: 80 - DNA, 24 bp, Artificial Sequence
  Description of Artificial Sequence Synthetic oligonucleotide
tttaaatgccggcgacggtggtgc

SEQ ID NO: 81 - DNA, 22 bp, Artificial Sequence
  Description of Artificial Sequence Synthetic oligonucleotide
gcgccaccgtcgccgcatttgg

SEQ ID NO: 82 - DNA, 23 bp, Artificial Sequence
  Description of Artificial Sequence Synthetic oligonucleotide
gcaccaccgtcgccggcatttgg

SEQ ID NO: 83 - DNA, 20 bp, Artificial Sequence
  Description of Artificial Sequence Synthetic oligonucleotide
gcaccaccgtcgccggcatt

SEQ ID NO: 84 - DNA, 20 bp, Artificial Sequence
  Description of Artificial Sequence Synthetic oligonucleotide
gatgccgttcttctgcttgt

SEQ ID NO: 85 - DNA, 22 bp, Artificial Sequence
  Description of Artificial Sequence Synthetic oligonucleotide
gatgccgttctttgcttgtcgg

SEQ ID NO: 86 - DNA, 22 bp, Artificial Sequence
  Description of Artificial Sequence Synthetic oligonucleotide
gatggcgtcttctgcttttcgg

SEQ ID NO: 87 - DNA, 23 bp, Artificial Sequence
  Description of Artificial Sequence Synthetic oligonucleotide
gatgccgttcttctgcttgtcgg

SEQ ID NO: 88 - DNA, 24 bp, Artificial Sequence
  Description of Artificial Sequence Synthetic oligonucleotide
tgggacaagcagaagaacggcatc

SEQ ID NO: 89 - DNA, 24 bp, Artificial Sequence
  Description of Artificial Sequence Synthetic oligonucleotide
ttgtacaagcagaagaacggcatc

SEQ ID NO: 90 - DNA, 24 bp, Artificial Sequence
  Description of Artificial Sequence Synthetic oligonucleotide
ttttccaagcagaagaacggcatc

SEQ ID NO: 91 - DNA, 24 bp, Artificial Sequence
  Description of Artificial Sequence Synthetic oligonucleotide
tttgcaaagcagaagaacggcatc

SEQ ID NO: 92 - DNA, 24 bp, Artificial Sequence
  Description of Artificial Sequence Synthetic oligonucleotide
tttgaacagcagaagaacggcatc

SEQ ID NO: 93 - DNA, 24 bp, Artificial Sequence
  Description of Artificial Sequence Synthetic oligonucleotide
tttgacccgcagaagaacggcatc

SEQ ID NO: 94 - DNA, 24 bp, Artificial Sequence
  Description of Artificial Sequence Synthetic oligonucleotide
tttgacactcagaagaacggcatc

SEQ ID NO: 95 - DNA, 24 bp, Artificial Sequence
  Description of Artificial Sequence Synthetic oligonucleotide
tttgacaataagaagaacggcatc

SEQ ID NO: 96 - DNA, 24 bp, Artificial Sequence
  Description of Artificial Sequence Synthetic oligonucleotide
tttgacaagacgaagaacggcatc

SEQ ID NO: 97 - DNA, 24 bp, Artificial Sequence
  Description of Artificial Sequence Synthetic oligonucleotide
tttgacaagcctaagaacggcatc

SEQ ID NO: 98 - DNA, 24 bp, Artificial Sequence
  Description of Artificial Sequence Synthetic oligonucleotide
tttgacaagcatcagaacggcatc

SEQ ID NO: 99 - DNA, 24 bp, Artificial Sequence
  Description of Artificial Sequence Synthetic oligonucleotide
tttgacaagcagccgaacggcatc

SEQ ID NO: 100 - DNA, 24 bp, Artificial Sequence
  Description of Artificial Sequence Synthetic oligonucleotide
tttgacaagcagactaacggcatc

SEQ ID NO: 101 - DNA, 24 bp, Artificial Sequence
  Description of Artificial Sequence Synthetic oligonucleotide
tttgacaagcagaatcacggcatc

SEQ ID NO: 102 - DNA, 24 bp, Artificial Sequence
  Description of Artificial Sequence Synthetic oligonucleotide
tttgacaagcagaagcccggcatc

SEQ ID NO: 103 - DNA, 24 bp, Artificial Sequence
  Description of Artificial Sequence Synthetic oligonucleotide
tttgacaagcagaagacaggcatc

SEQ ID NO: 104 - DNA, 24 bp, Artificial Sequence
  Description of Artificial Sequence Synthetic oligonucleotide
tttgacaagcagaagaaatgcatc

SEQ ID NO: 105 - DNA, 24 bp, Artificial Sequence
  Description of Artificial Sequence Synthetic oligonucleotide
tttgacaagcagaagaacttcatc

SEQ ID NO: 106 - DNA, 24 bp, Artificial Sequence
  Description of Artificial Sequence Synthetic oligonucleotide
tttgacaagcagaagaacgtaatc

SEQ ID NO: 107 - DNA, 23 bp, Artificial Sequence
  Description of Artificial Sequence Synthetic oligonucleotide
tttgcaagcagaagaacggcatc

SEQ ID NO: 108 - DNA, 23 bp, Artificial Sequence
  Description of Artificial Sequence Synthetic oligonucleotide
tttgaaagcagaagaacggcatc

SEQ ID NO: 109 - DNA, 23 bp, Artificial Sequence
  Description of Artificial Sequence Synthetic oligonucleotide
tttgacagcagaagaacggcatc

SEQ ID NO: 110 - DNA, 23 bp, Artificial Sequence
  Description of Artificial Sequence Synthetic oligonucleotide
tttgacaacagaagaacggcatc

SEQ ID NO: 111 - DNA, 23 bp, Artificial Sequence
  Description of Artificial Sequence Synthetic oligonucleotide
tttgacaagagaagaacggcatc

SEQ ID NO: 112 - DNA, 23 bp, Artificial Sequence
  Description of Artificial Sequence Synthetic oligonucleotide
tttgacaagcgaagaacggcatc

SEQ ID NO: 113 - DNA, 23 bp, Artificial Sequence
  Description of Artificial Sequence Synthetic oligonucleotide
tttgacaagcaaagaacggcatc

SEQ ID NO: 114 - DNA, 23 bp, Artificial Sequence
  Description of Artificial Sequence Synthetic oligonucleotide
tttgacaagcagagaacggcatc

SEQ ID NO: 115 - DNA, 23 bp, Artificial Sequence
  Description of Artificial Sequence Synthetic oligonucleotide
tttgacaagcagaaaacggcatc

SEQ ID NO: 116 - DNA, 23 bp, Artificial Sequence
  Description of Artificial Sequence Synthetic oligonucleotide
tttgacaagcagaagacggcatc

SEQ ID NO: 117 - DNA, 23 bp, Artificial Sequence
  Description of Artificial Sequence Synthetic oligonucleotide
tttgacaagcagaagaaggcatc

SEQ ID NO: 118 - DNA, 23 bp, Artificial Sequence
  Description of Artificial Sequence Synthetic oligonucleotide
tttgacaagcagaagaacgcatc

SEQ ID NO: 119 - DNA, 23 bp, Artificial Sequence
  Description of Artificial Sequence Synthetic oligonucleotide
tttgacaagcagaagaacggatc

SEQ ID NO: 120 - DNA, 23 bp, Artificial Sequence
  Description of Artificial Sequence Synthetic oligonucleotide
tttgacaagcagaagaacggcac

SEQ ID NO: 121 - DNA, 23 bp, Artificial Sequence
  Description of Artificial Sequence Synthetic oligonucleotide
ttttcatattgttgacgtctcac

SEQ ID NO: 122 - DNA, 23 bp, Artificial Sequence
  Description of Artificial Sequence Synthetic oligonucleotide
ttttcctattgttgacgtctcac

SEQ ID NO: 123 - DNA, 23 bp, Artificial Sequence
  Description of Artificial Sequence Synthetic oligonucleotide
ttttccaattgttgacgtctcac

SEQ ID NO: 124 - DNA, 23 bp, Artificial Sequence
  Description of Artificial Sequence Synthetic oligonucleotide
ttttccatttgttgacgtctcac

SEQ ID NO: 125 - DNA, 23 bp, Artificial Sequence
  Description of Artificial Sequence Synthetic oligonucleotide
ttttccatatgttgacgtctcac

SEQ ID NO: 126 - DNA, 23 bp, Artificial Sequence
  Description of Artificial Sequence Synthetic oligonucleotide
ttttccatattttgacgtctcac

SEQ ID NO: 127 - DNA, 23 bp, Artificial Sequence
  Description of Artificial Sequence Synthetic oligonucleotide
ttttccatattgtgacgtctcac

SEQ ID NO: 128 - DNA, 23 bp, Artificial Sequence
  Description of Artificial Sequence Synthetic oligonucleotide
ttttccatattgttacgtctcac

SEQ ID NO: 129 - DNA, 23 bp, Artificial Sequence
  Description of Artificial Sequence Synthetic oligonucleotide
ttttccatattgttgcgtctcac

SEQ ID NO: 130 - DNA, 23 bp, Artificial Sequence
  Description of Artificial Sequence Synthetic oligonucleotide
ttttccatattgttgagtctcac

SEQ ID NO: 131 - DNA, 23 bp, Artificial Sequence
  Description of Artificial Sequence Synthetic oligonucleotide
ttttccatattgttgactctcac

SEQ ID NO: 132 - DNA, 23 bp, Artificial Sequence
  Description of Artificial Sequence Synthetic oligonucleotide
ttttccatattgttgacgctcac

SEQ ID NO: 133 - DNA, 23 bp, Artificial Sequence
  Description of Artificial Sequence Synthetic oligonucleotide
ttttccatattgttgacgttcac

SEQ ID NO: 134 - DNA, 23 bp, Artificial Sequence
  Description of Artificial Sequence Synthetic oligonucleotide
ttttccatattgttgacgtccac

SEQ ID NO: 135 - DNA, 24 bp, Artificial Sequence
  Description of Artificial Sequence Synthetic oligonucleotide
tggtccatattgttgacgtctcac

SEQ ID NO: 136 - DNA, 24 bp, Artificial Sequence
  Description of Artificial Sequence Synthetic oligonucleotide
ttggccatattgttgacgtctcac

SEQ ID NO: 137 - DNA, 24 bp, Artificial Sequence
  Description of Artificial Sequence Synthetic oligonucleotide
tttgacatattgttgacgtctcac

SEQ ID NO: 138 - DNA, 24 bp, Artificial Sequence
  Description of Artificial Sequence Synthetic oligonucleotide
ttttaaatattgttgacgtctcac

SEQ ID NO: 139 - DNA, 24 bp, Artificial Sequence
  Description of Artificial Sequence Synthetic oligonucleotide
ttttcactattgttgacgtctcac

SEQ ID NO: 140 - DNA, 24 bp, Artificial Sequence
  Description of Artificial Sequence Synthetic oligonucleotide
ttttcccgattgttgacgtctcac

SEQ ID NO: 141 - DNA, 24 bp, Artificial Sequence
  Description of Artificial Sequence Synthetic oligonucleotide
ttttccagcttgttgacgtctcac

SEQ ID NO: 142 - DNA, 24 bp, Artificial Sequence
  Description of Artificial Sequence Synthetic oligonucleotide
ttttccatcgtgttgacgtctcac

SEQ ID NO: 143 - DNA, 24 bp, Artificial Sequence
  Description of Artificial Sequence Synthetic oligonucleotide
ttttccatagggttgacgtctcac

SEQ ID NO: 144 - DNA, 24 bp, Artificial Sequence
  Description of Artificial Sequence Synthetic oligonucleotide
ttttccatatgtttgacgtctcac

SEQ ID NO: 145 - DNA, 24 bp, Artificial Sequence
  Description of Artificial Sequence Synthetic oligonucleotide
ttttccatatttgtgacgtctcac

SEQ ID NO: 146 - DNA, 24 bp, Artificial Sequence
  Description of Artificial Sequence Synthetic oligonucleotide
ttttccatattggggacgtctcac

SEQ ID NO: 147 - DNA, 24 bp, Artificial Sequence
  Description of Artificial Sequence Synthetic oligonucleotide
ttttccatattgtgtacgtctcac

SEQ ID NO: 148 - DNA, 24 bp, Artificial Sequence
  Description of Artificial Sequence Synthetic oligonucleotide
ttttccatattgtttccgtctcac

SEQ ID NO: 149 - DNA, 24 bp, Artificial Sequence
  Description of Artificial Sequence Synthetic oligonucleotide
ttttccatattgttgcagtctcac

SEQ ID NO: 150 - DNA, 24 bp, Artificial Sequence
  Description of Artificial Sequence Synthetic oligonucleotide
ttttccatattgttgaattctcac

SEQ ID NO: 151 - DNA, 24 bp, Artificial Sequence
  Description of Artificial Sequence Synthetic oligonucleotide
ttttccatattgttgactgctcac

SEQ ID NO: 152 - DNA, 24 bp, Artificial Sequence
  Description of Artificial Sequence Synthetic oligonucleotide
ttttccatattgttgacggatcac

SEQ ID NO: 153 - DNA, 24 bp, Artificial Sequence
  Description of Artificial Sequence Synthetic oligonucleotide
ttttccatattgttgacgtagcac

SEQ ID NO: 154 - DNA, 23 bp, Artificial Sequence
  Description of Artificial Sequence Synthetic oligonucleotide
tttacggcaagctgcccgtgccc

SEQ ID NO: 155 - DNA, 23 bp, Artificial Sequence
  Description of Artificial Sequence Synthetic oligonucleotide
tttaccgcaagctgcccgtgccc

SEQ ID NO: 156 - DNA, 23 bp, Artificial Sequence
  Description of Artificial Sequence Synthetic oligonucleotide
tttaccggaagctgcccgtgccc

SEQ ID NO: 157 - DNA, 23 bp, Artificial Sequence
  Description of Artificial Sequence Synthetic oligonucleotide
tttaccggcagctgcccgtgccc

SEQ ID NO: 158 - DNA, 23 bp, Artificial Sequence
  Description of Artificial Sequence Synthetic oligonucleotide
tttaccggcaactgcccgtgccc

SEQ ID NO: 159 - DNA, 23 bp, Artificial Sequence
  Description of Artificial Sequence Synthetic oligonucleotide
tttaccggcaagtgcccgtgccc

SEQ ID NO: 160 - DNA, 23 bp, Artificial Sequence
  Description of Artificial Sequence Synthetic oligonucleotide
tttaccggcaagcgcccgtgccc

SEQ ID NO: 161 - DNA, 23 bp, Artificial Sequence
  Description of Artificial Sequence Synthetic oligonucleotide
tttaccggcaagctcccgtgccc

SEQ ID NO: 162 - DNA, 23 bp, Artificial Sequence
  Description of Artificial Sequence Synthetic oligonucleotide
tttaccggcaagctgccgtgccc

SEQ ID NO: 163 - DNA, 23 bp, Artificial Sequence
  Description of Artificial Sequence Synthetic oligonucleotide
tttaccggcaagctgccctgccc

SEQ ID NO: 164 - DNA, 23 bp, Artificial Sequence
  Description of Artificial Sequence Synthetic oligonucleotide
tttaccggcaagctgcccggccc

SEQ ID NO: 165 - DNA, 23 bp, Artificial Sequence
  Description of Artificial Sequence Synthetic oligonucleotide
tttaccggcaagctgcccgtccc

SEQ ID NO: 166 - DNA, 24 bp, Artificial Sequence
  Description of Artificial Sequence Synthetic oligonucleotide
tggaccggcaagctgcccgtgccc

SEQ ID NO: 167 - DNA, 24 bp, Artificial Sequence
  Description of Artificial Sequence Synthetic oligonucleotide
ttgcccggcaagctgcccgtgccc

SEQ ID NO: 168 - DNA, 24 bp, Artificial Sequence
  Description of Artificial Sequence Synthetic oligonucleotide
tttcacggcaagctgcccgtgccc

SEQ ID NO: 169 - DNA, 24 bp, Artificial Sequence
  Description of Artificial Sequence Synthetic oligonucleotide
tttaaaggcaagctgcccgtgccc

SEQ ID NO: 170 - DNA, 24 bp, Artificial Sequence
  Description of Artificial Sequence Synthetic oligonucleotide
tttacatgcaagctgcccgtgccc

SEQ ID NO: 171 - DNA, 24 bp, Artificial Sequence
  Description of Artificial Sequence Synthetic oligonucleotide
tttaccttcaagctgcccgtgccc

SEQ ID NO: 172 - DNA, 24 bp, Artificial Sequence
  Description of Artificial Sequence Synthetic oligonucleotide
tttaccgtaaagctgcccgtgccc

SEQ ID NO: 173 - DNA, 24 bp, Artificial Sequence
  Description of Artificial Sequence Synthetic oligonucleotide
tttaccggacagctgcccgtgccc

SEQ ID NO: 174 - DNA, 24 bp, Artificial Sequence
  Description of Artificial Sequence Synthetic oligonucleotide
tttaccggcccgctgcccgtgccc

SEQ ID NO: 175 - DNA, 24 bp, Artificial Sequence
  Description of Artificial Sequence Synthetic oligonucleotide
tttaccggcactctgcccgtgccc

SEQ ID NO: 176 - DNA, 24 bp, Artificial Sequence
  Description of Artificial Sequence Synthetic oligonucleotide
tttaccggcaatatgcccgtgccc

SEQ ID NO: 177 - DNA, 24 bp, Artificial Sequence
  Description of Artificial Sequence Synthetic oligonucleotide
tttaccggcaagaggcccgtgccc

SEQ ID NO: 178 - DNA, 24 bp, Artificial Sequence
  Description of Artificial Sequence Synthetic oligonucleotide
tttaccggcaagcgtcccgtgccc

SEQ ID NO: 179 - DNA, 24 bp, Artificial Sequence
  Description of Artificial Sequence Synthetic oligonucleotide
tttaccggcaagcttaccgtgccc

SEQ ID NO: 180 - DNA, 24 bp, Artificial Sequence
  Description of Artificial Sequence Synthetic oligonucleotide
tttaccggcaagctgaacgtgccc

SEQ ID NO: 181 - DNA, 24 bp, Artificial Sequence
  Description of Artificial Sequence Synthetic oligonucleotide
tttaccggcaagctgcaagtgccc

SEQ ID NO: 182 - DNA, 24 bp, Artificial Sequence
  Description of Artificial Sequence Synthetic oligonucleotide
tttaccggcaagctgccattgccc

SEQ ID NO: 183 - DNA, 24 bp, Artificial Sequence
  Description of Artificial Sequence Synthetic oligonucleotide
tttaccggcaagctgccctggccc

SEQ ID NO: 184 - DNA, 24 bp, Artificial Sequence
  Description of Artificial Sequence Synthetic oligonucleotide
tttaccggcaagctgcccggtccc

SEQ ID NO: 185 - DNA, 23 bp, Artificial Sequence
  Description of Artificial Sequence Synthetic oligonucleotide
tttacggcaagctgcccgtgccc

SEQ ID NO: 186 - DNA, 23 bp, Artificial Sequence
  Description of Artificial Sequence Synthetic oligonucleotide
tttaccgcaagctgcccgtgccc

SEQ ID NO: 187 - DNA, 23 bp, Artificial Sequence
  Description of Artificial Sequence Synthetic oligonucleotide
tttaccggaagctgcccgtgccc

SEQ ID NO: 188 - DNA, 23 bp, Artificial Sequence
  Description of Artificial Sequence Synthetic oligonucleotide
tttaccggcagctgcccgtgccc

SEQ ID NO: 189 - DNA, 23 bp, Artificial Sequence
  Description of Artificial Sequence Synthetic oligonucleotide
tttaccggcaactgcccgtgccc

SEQ ID NO: 190 - DNA, 23 bp, Artificial Sequence
  Description of Artificial Sequence Synthetic oligonucleotide
tttaccggcaagtgcccgtgccc

SEQ ID NO: 191 - DNA, 23 bp, Artificial Sequence
  Description of Artificial Sequence Synthetic oligonucleotide
tttaccggcaagcgcccgtgccc

SEQ ID NO: 192 - DNA, 23 bp, Artificial Sequence
  Description of Artificial Sequence Synthetic oligonucleotide
tttaccggcaagctcccgtgccc

SEQ ID NO: 193 - DNA, 23 bp, Artificial Sequence
  Description of Artificial Sequence Synthetic oligonucleotide
tttaccggcaagctgccgtgccc

SEQ ID NO: 194 - DNA, 23 bp, Artificial Sequence
  Description of Artificial Sequence Synthetic oligonucleotide
tttaccggcaagctgccctgccc

SEQ ID NO: 195 - DNA, 23 bp, Artificial Sequence
  Description of Artificial Sequence Synthetic oligonucleotide
tttaccggcaagctgcccggccc

SEQ ID NO: 196 - DNA, 23 bp, Artificial Sequence
  Description of Artificial Sequence Synthetic oligonucleotide
tttaccggcaagctgcccgtccc

SEQ ID NO: 197 - DNA, 24 bp, Artificial Sequence
  Description of Artificial Sequence Synthetic oligonucleotide
tggaccggcaagctgcccgtgccc

SEQ ID NO: 198 - DNA, 24 bp, Artificial Sequence
  Description of Artificial Sequence Synthetic oligonucleotide
ttgcccggcaagctgcccgtgccc

SEQ ID NO: 199 - DNA, 24 bp, Artificial Sequence
  Description of Artificial Sequence Synthetic oligonucleotide
tttcacggcaagctgcccgtgccc

SEQ ID NO: 200 - DNA, 24 bp, Artificial Sequence
  Description of Artificial Sequence Synthetic oligonucleotide
tttaaaggcaagctgcccgtgccc

SEQ ID NO: 201 - DNA, 24 bp, Artificial Sequence
  Description of Artificial Sequence Synthetic oligonucleotide
tttacatgcaagctgcccgtgccc

SEQ ID NO: 202 - DNA, 24 bp, Artificial Sequence
  Description of Artificial Sequence Synthetic oligonucleotide
tttaccttcaagctgcccgtgccc

SEQ ID NO: 203 - DNA, 24 bp, Artificial Sequence
  Description of Artificial Sequence Synthetic oligonucleotide
tttaccgtaaagctgcccgtgccc

SEQ ID NO: 204 - DNA, 24 bp, Artificial Sequence
  Description of Artificial Sequence Synthetic oligonucleotide
tttaccggacagctgcccgtgccc

SEQ ID NO: 205 - DNA, 24 bp, Artificial Sequence
  Description of Artificial Sequence Synthetic oligonucleotide
tttaccggcccgctgcccgtgccc

SEQ ID NO: 206 - DNA, 24 bp, Artificial Sequence
  Description of Artificial Sequence Synthetic oligonucleotide
tttaccggcactctgcccgtgccc

SEQ ID NO: 207 - DNA, 24 bp, Artificial Sequence
  Description of Artificial Sequence Synthetic oligonucleotide
tttaccggcaatatgcccgtgccc

SEQ ID NO: 208 - DNA, 24 bp, Artificial Sequence
  Description of Artificial Sequence Synthetic oligonucleotide
tttaccggcaagaggcccgtgccc

SEQ ID NO: 209 - DNA, 24 bp, Artificial Sequence
  Description of Artificial Sequence Synthetic oligonucleotide
tttaccggcaagcgtcccgtgccc

SEQ ID NO: 210 - DNA, 24 bp, Artificial Sequence
  Description of Artificial Sequence Synthetic oligonucleotide
tttaccggcaagcttaccgtgccc

SEQ ID NO: 211 - DNA, 24 bp, Artificial Sequence
  Description of Artificial Sequence Synthetic oligonucleotide
tttaccggcaagctgaacgtgccc

SEQ ID NO: 212 - DNA, 24 bp, Artificial Sequence
  Description of Artificial Sequence Synthetic oligonucleotide
tttaccggcaagctgcaagtgccc

SEQ ID NO: 213 - DNA, 24 bp, Artificial Sequence
  Description of Artificial Sequence Synthetic oligonucleotide
tttaccggcaagctgccattgccc

SEQ ID NO: 214 - DNA, 24 bp, Artificial Sequence
  Description of Artificial Sequence Synthetic oligonucleotide
tttaccggcaagctgccctggccc

SEQ ID NO: 215 - DNA, 24 bp, Artificial Sequence
  Description of Artificial Sequence Synthetic oligonucleotide
tttaccggcaagctgcccggtccc

SEQ ID NO: 216 - DNA, 23 bp, Artificial Sequence
  Description of Artificial Sequence Synthetic oligonucleotide
tttaatgccggcgacggtggtgc

SEQ ID NO: 217 - DNA, 23 bp, Artificial Sequence
  Description of Artificial Sequence Synthetic oligonucleotide
tttaaagccggcgacggtggtgc

SEQ ID NO: 218 - DNA, 23 bp, Artificial Sequence
  Description of Artificial Sequence Synthetic oligonucleotide
tttaaatccggcgacggtggtgc

SEQ ID NO: 219 - DNA, 23 bp, Artificial Sequence
  Description of Artificial Sequence Synthetic oligonucleotide
tttaaatgcggcgacggtggtgc

SEQ ID NO: 220 - DNA, 23 bp, Artificial Sequence
  Description of Artificial Sequence Synthetic oligonucleotide
tttaaatgccgcgacggtggtgc

SEQ ID NO: 221 - DNA, 23 bp, Artificial Sequence
  Description of Artificial Sequence Synthetic oligonucleotide
tttaaatgccgggacggtggtgc

SEQ ID NO: 222 - DNA, 23 bp, Artificial Sequence
  Description of Artificial Sequence Synthetic oligonucleotide
tttaaatgccggcacggtggtgc

SEQ ID NO: 223 - DNA, 23 bp, Artificial Sequence
  Description of Artificial Sequence Synthetic oligonucleotide
tttaaatgccggcgcggtggtgc

SEQ ID NO: 224 - DNA, 23 bp, Artificial Sequence
  Description of Artificial Sequence Synthetic oligonucleotide
tttaaatgccggcgaggtggtgc

SEQ ID NO: 225 - DNA, 23 bp, Artificial Sequence
  Description of Artificial Sequence Synthetic oligonucleotide
tttaaatgccggcgacgtggtgc

SEQ ID NO: 226 - DNA, 23 bp, Artificial Sequence
  Description of Artificial Sequence Synthetic oligonucleotide
tttaaatgccggcgacggggtgc

SEQ ID NO: 227 - DNA, 23 bp, Artificial Sequence
  Description of Artificial Sequence Synthetic oligonucleotide
tttaaatgccggcgacggtgtgc

SEQ ID NO: 228 - DNA, 24 bp, Artificial Sequence
  Description of Artificial Sequence Synthetic oligonucleotide
tggaaatgccggcgacggtggtgc

SEQ ID NO: 229 - DNA, 24 bp, Artificial Sequence
  Description of Artificial Sequence Synthetic oligonucleotide
ttgcaatgccggcgacggtggtgc

SEQ ID NO: 230 - DNA, 24 bp, Artificial Sequence
  Description of Artificial Sequence Synthetic oligonucleotide
tttccatgccggcgacggtggtgc

SEQ ID NO: 231 - DNA, 24 bp, Artificial Sequence
  Description of Artificial Sequence Synthetic oligonucleotide
tttacctgccggcgacggtggtgc

SEQ ID NO: 232 - DNA, 24 bp, Artificial Sequence
  Description of Artificial Sequence Synthetic oligonucleotide
tttaacggccggcgacggtggtgc

SEQ ID NO: 233 - DNA, 24 bp, Artificial Sequence
  Description of Artificial Sequence Synthetic oligonucleotide
tttaaagtccggcgacggtggtgc

SEQ ID NO: 234 - DNA, 24 bp, Artificial Sequence
  Description of Artificial Sequence Synthetic oligonucleotide
tttaaattacggcgacggtggtgc

SEQ ID NO: 235 - DNA, 24 bp, Artificial Sequence
  Description of Artificial Sequence Synthetic oligonucleotide
tttaaatgaaggcgacggtggtgc

SEQ ID NO: 236 - DNA, 24 bp, Artificial Sequence
  Description of Artificial Sequence Synthetic oligonucleotide
tttaaatgcatgcgacggtggtgc

SEQ ID NO: 237 - DNA, 24 bp, Artificial Sequence
  Description of Artificial Sequence Synthetic oligonucleotide
tttaaatgccttcgacggtggtgc

SEQ ID NO: 238 - DNA, 24 bp, Artificial Sequence
  Description of Artificial Sequence Synthetic oligonucleotide
tttaaatgccgtagacggtggtgc

SEQ ID NO: 239 - DNA, 24 bp, Artificial Sequence
  Description of Artificial Sequence Synthetic oligonucleotide
tttaaatgccggatacggtggtgc

SEQ ID NO: 240 - DNA, 24 bp, Artificial Sequence
  Description of Artificial Sequence Synthetic oligonucleotide
tttaaatgccggctccggtggtgc

SEQ ID NO: 241 - DNA, 24 bp, Artificial Sequence
  Description of Artificial Sequence Synthetic oligonucleotide
tttaaatgccggcgcaggtggtgc

SEQ ID NO: 242 - DNA, 24 bp, Artificial Sequence
  Description of Artificial Sequence Synthetic oligonucleotide
tttaaatgccggcgaatgtggtgc

SEQ ID NO: 243 - DNA, 24 bp, Artificial Sequence
  Description of Artificial Sequence Synthetic oligonucleotide
tttaaatgccggcgactttggtgc

SEQ ID NO: 244 - DNA, 24 bp, Artificial Sequence
  Description of Artificial Sequence Synthetic oligonucleotide
tttaaatgccggcgacgtgggtgc

SEQ ID NO: 245 - DNA, 24 bp, Artificial Sequence
  Description of Artificial Sequence Synthetic oligonucleotide
tttaaatgccggcgacgggtgtgc

SEQ ID NO: 246 - DNA, 24 bp, Artificial Sequence
  Description of Artificial Sequence Synthetic oligonucleotide
tttaaatgccggcgacggttttgc

SEQ ID NO: 247 - DNA, 23 bp, Artificial Sequence
  Description of Artificial Sequence Synthetic oligonucleotide
ttttcatattgttgacgtctcac

SEQ ID NO: 248 - DNA, 23 bp, Artificial Sequence
  Description of Artificial Sequence Synthetic oligonucleotide
ttttcctattgttgacgtctcac

SEQ ID NO: 249 - DNA, 23 bp, Artificial Sequence
  Description of Artificial Sequence Synthetic oligonucleotide
ttttccaattgttgacgtctcac

SEQ ID NO: 250 - DNA, 23 bp, Artificial Sequence
  Description of Artificial Sequence Synthetic oligonucleotide
ttttccatttgttgacgtctcac

SEQ ID NO: 251 - DNA, 23 bp, Artificial Sequence
  Description of Artificial Sequence Synthetic oligonucleotide
ttttccatatgttgacgtctcac

SEQ ID NO: 252 - DNA, 23 bp, Artificial Sequence
  Description of Artificial Sequence Synthetic oligonucleotide
ttttccatattttgacgtctcac

SEQ ID NO: 253 - DNA, 23 bp, Artificial Sequence
  Description of Artificial Sequence Synthetic oligonucleotide
ttttccatattgtgacgtctcac

SEQ ID NO: 254 - DNA, 23 bp, Artificial Sequence
  Description of Artificial Sequence Synthetic oligonucleotide
ttttccatattgttacgtctcac

SEQ ID NO: 255 - DNA, 23 bp, Artificial Sequence
  Description of Artificial Sequence Synthetic oligonucleotide
ttttccatattgttgcgtctcac

SEQ ID NO: 256 - DNA, 23 bp, Artificial Sequence
  Description of Artificial Sequence Synthetic oligonucleotide
ttttccatattgttgagtctcac

SEQ ID NO: 257 - DNA, 23 bp, Artificial Sequence
  Description of Artificial Sequence Synthetic oligonucleotide
ttttccatattgttgactctcac

SEQ ID NO: 258 - DNA, 23 bp, Artificial Sequence
  Description of Artificial Sequence Synthetic oligonucleotide
ttttccatattgttgacgctcac

SEQ ID NO: 259 - DNA, 23 bp, Artificial Sequence
  Description of Artificial Sequence Synthetic oligonucleotide
ttttccatattgttgacgttcac

SEQ ID NO: 260 - DNA, 23 bp, Artificial Sequence
  Description of Artificial Sequence Synthetic oligonucleotide
ttttccatattgttgacgtccac

SEQ ID NO: 261 - DNA, 24 bp, Artificial Sequence
  Description of Artificial Sequence Synthetic oligonucleotide
tggtccatattgttgacgtctcac

SEQ ID NO: 262 - DNA, 24 bp, Artificial Sequence
  Description of Artificial Sequence Synthetic oligonucleotide
ttggccatattgttgacgtctcac

SEQ ID NO: 263 - DNA, 24 bp, Artificial Sequence
  Description of Artificial Sequence Synthetic oligonucleotide
tttgacatattgttgacgtctcac

SEQ ID NO: 264 - DNA, 24 bp, Artificial Sequence
  Description of Artificial Sequence Synthetic oligonucleotide
ttttcactattgttgacgtctcac

SEQ ID NO: 265 - DNA, 24 bp, Artificial Sequence
  Description of Artificial Sequence Synthetic oligonucleotide
ttttcccgattgttgacgtctcac

SEQ ID NO: 266 - DNA, 24 bp, Artificial Sequence
  Description of Artificial Sequence Synthetic oligonucleotide
ttttccagcttgttgacgtctcac

SEQ ID NO: 267 - DNA, 24 bp, Artificial Sequence
  Description of Artificial Sequence Synthetic oligonucleotide
ttttccatcgtgttgacgtctcac

SEQ ID NO: 268 - DNA, 24 bp, Artificial Sequence
  Description of Artificial Sequence Synthetic oligonucleotide
ttttccatagggttgacgtctcac

SEQ ID NO: 269 - DNA, 24 bp, Artificial Sequence
  Description of Artificial Sequence Synthetic oligonucleotide
ttttccatatgtttgacgtctcac

SEQ ID NO: 270 - DNA, 24 bp, Artificial Sequence
  Description of Artificial Sequence Synthetic oligonucleotide
ttttccatatttgtgacgtctcac

SEQ ID NO: 271 - DNA, 24 bp, Artificial Sequence
  Description of Artificial Sequence Synthetic oligonucleotide
ttttccatattggggacgtctcac

SEQ ID NO: 272 - DNA, 24 bp, Artificial Sequence
  Description of Artificial Sequence Synthetic oligonucleotide
ttttccatattgtgtacgtctcac

SEQ ID NO: 273 - DNA, 24 bp, Artificial Sequence
  Description of Artificial Sequence Synthetic oligonucleotide
ttttccatattgtttccgtctcac

SEQ ID NO: 274 - DNA, 24 bp, Artificial Sequence
  Description of Artificial Sequence Synthetic oligonucleotide
ttttccatattgttgcagtctcac

SEQ ID NO: 275 - DNA, 24 bp, Artificial Sequence
  Description of Artificial Sequence Synthetic oligonucleotide
ttttccatattgttgaattctcac

SEQ ID NO: 276 - DNA, 24 bp, Artificial Sequence
  Description of Artificial Sequence Synthetic oligonucleotide
ttttccatattgttgactgctcac

SEQ ID NO: 277 - DNA, 24 bp, Artificial Sequence
  Description of Artificial Sequence Synthetic oligonucleotide
ttttccatattgttgacggatcac

SEQ ID NO: 278 - DNA, 24 bp, Artificial Sequence
  Description of Artificial Sequence Synthetic oligonucleotide
ttttccatattgttgacgtagcac

SEQ ID NO: 279 - DNA, 23 bp, Artificial Sequence
  Description of Artificial Sequence Synthetic oligonucleotide
tttacggcaagctgcccgtgccc

SEQ ID NO: 280 - DNA, 23 bp, Artificial Sequence
  Description of Artificial Sequence Synthetic oligonucleotide
tttaccgcaagctgcccgtgccc

SEQ ID NO: 281 - DNA, 23 bp, Artificial Sequence
  Description of Artificial Sequence Synthetic oligonucleotide
tttaccggaagctgcccgtgccc

SEQ ID NO: 282 - DNA, 23 bp, Artificial Sequence
  Description of Artificial Sequence Synthetic oligonucleotide
tttaccggcagctgcccgtgccc

SEQ ID NO: 283 - DNA, 23 bp, Artificial Sequence
  Description of Artificial Sequence Synthetic oligonucleotide
tttaccggcaactgcccgtgccc

SEQ ID NO: 284 - DNA, 23 bp, Artificial Sequence
  Description of Artificial Sequence Synthetic oligonucleotide
tttaccggcaagtgcccgtgccc

SEQ ID NO: 285 - DNA, 23 bp, Artificial Sequence
  Description of Artificial Sequence Synthetic oligonucleotide
tttaccggcaagcgcccgtgccc

SEQ ID NO: 286 - DNA, 23 bp, Artificial Sequence
  Description of Artificial Sequence Synthetic oligonucleotide
tttaccggcaagctcccgtgccc

SEQ ID NO: 287 - DNA, 23 bp, Artificial Sequence
  Description of Artificial Sequence Synthetic oligonucleotide
tttaccggcaagctgccgtgccc

SEQ ID NO: 288 - DNA, 23 bp, Artificial Sequence
  Description of Artificial Sequence Synthetic oligonucleotide
tttaccggcaagctgccctgccc

SEQ ID NO: 289 - DNA, 23 bp, Artificial Sequence
  Description of Artificial Sequence Synthetic oligonucleotide
tttaccggcaagctgcccggccc

SEQ ID NO: 290 - DNA, 23 bp, Artificial Sequence
  Description of Artificial Sequence Synthetic oligonucleotide
tttaccggcaagctgcccgtccc

SEQ ID NO: 291 - DNA, 24 bp, Artificial Sequence
  Description of Artificial Sequence Synthetic oligonucleotide
tggaccggcaagctgcccgtgccc

SEQ ID NO: 292 - DNA, 24 bp, Artificial Sequence
  Description of Artificial Sequence Synthetic oligonucleotide
ttgcccggcaagctgcccgtgccc

SEQ ID NO: 293 - DNA, 24 bp, Artificial Sequence
  Description of Artificial Sequence Synthetic oligonucleotide
tttcacggcaagctgcccgtgccc

SEQ ID NO: 294 - DNA, 24 bp, Artificial Sequence
  Description of Artificial Sequence Synthetic oligonucleotide
tttaaaggcaagctgcccgtgccc

SEQ ID NO: 295 - DNA, 24 bp, Artificial Sequence
  Description of Artificial Sequence Synthetic oligonucleotide
tttacatgcaagctgcccgtgccc

SEQ ID NO: 296 - DNA, 24 bp, Artificial Sequence
  Description of Artificial Sequence Synthetic oligonucleotide
tttaccttcaagctgcccgtgccc

SEQ ID NO: 297 - DNA, 24 bp, Artificial Sequence
  Description of Artificial Sequence Synthetic oligonucleotide
tttaccgtaaagctgcccgtgccc

SEQ ID NO: 298 - DNA, 24 bp, Artificial Sequence
  Description of Artificial Sequence Synthetic oligonucleotide
tttaccggacagctgcccgtgccc

SEQ ID NO: 299 - DNA, 24 bp, Artificial Sequence
  Description of Artificial Sequence Synthetic oligonucleotide
tttaccggcccgctgcccgtgccc

SEQ ID NO: 300 - DNA, 24 bp, Artificial Sequence
  Description of Artificial Sequence Synthetic oligonucleotide
tttaccggcactctgcccgtgccc

SEQ ID NO: 301 - DNA, 24 bp, Artificial Sequence
  Description of Artificial Sequence Synthetic oligonucleotide
tttaccggcaatatgcccgtgccc

SEQ ID NO: 302 - DNA, 24 bp, Artificial Sequence
  Description of Artificial Sequence Synthetic oligonucleotide
tttaccggcaagaggcccgtgccc

SEQ ID NO: 303 - DNA, 24 bp, Artificial Sequence
  Description of Artificial Sequence Synthetic oligonucleotide
tttaccggcaagcgtcccgtgccc

SEQ ID NO: 304 - DNA, 24 bp, Artificial Sequence
  Description of Artificial Sequence Synthetic oligonucleotide
tttaccggcaagcttaccgtgccc

SEQ ID NO: 305 - DNA, 24 bp, Artificial Sequence
  Description of Artificial Sequence Synthetic oligonucleotide
tttaccggcaagctgaacgtgccc

SEQ ID NO: 306 - DNA, 24 bp, Artificial Sequence
  Description of Artificial Sequence Synthetic oligonucleotide
tttaccggcaagctgcaagtgccc

SEQ ID NO: 307 - DNA, 24 bp, Artificial Sequence
  Description of Artificial Sequence Synthetic oligonucleotide
tttaccggcaagctgccattgccc

SEQ ID NO: 308 - DNA, 24 bp, Artificial Sequence
  Description of Artificial Sequence Synthetic oligonucleotide
tttaccggcaagctgccctggccc

SEQ ID NO: 309 - DNA, 24 bp, Artificial Sequence
  Description of Artificial Sequence Synthetic oligonucleotide
tttaccggcaagctgcccggtccc

SEQ ID NO: 310 - DNA, 23 bp, Artificial Sequence
  Description of Artificial Sequence Synthetic oligonucleotide
tttgcaagcagaagaacggcatc

SEQ ID NO: 311 - DNA, 23 bp, Artificial Sequence
  Description of Artificial Sequence Synthetic oligonucleotide
tttgaaagcagaagaacggcatc

SEQ ID NO: 312 - DNA, 23 bp, Artificial Sequence
  Description of Artificial Sequence Synthetic oligonucleotide
tttgacagcagaagaacggcatc

SEQ ID NO: 313 - DNA, 23 bp, Artificial Sequence
  Description of Artificial Sequence Synthetic oligonucleotide
tttgacaacagaagaacggcatc

SEQ ID NO: 314 - DNA, 23 bp, Artificial Sequence
  Description of Artificial Sequence Synthetic oligonucleotide
tttgacaagagaagaacggcatc

SEQ ID NO: 315 - DNA, 23 bp, Artificial Sequence
  Description of Artificial Sequence Synthetic oligonucleotide
tttgacaagcgaagaacggcatc

SEQ ID NO: 316 - DNA, 23 bp, Artificial Sequence
  Description of Artificial Sequence Synthetic oligonucleotide
tttgacaagcaaagaacggcatc

SEQ ID NO: 317 - DNA, 23 bp, Artificial Sequence
  Description of Artificial Sequence Synthetic oligonucleotide
tttgacaagcagagaacggcatc

SEQ ID NO: 318 - DNA, 23 bp, Artificial Sequence
  Description of Artificial Sequence Synthetic oligonucleotide
tttgacaagcagaaaacggcatc

SEQ ID NO: 319 - DNA, 23 bp, Artificial Sequence
  Description of Artificial Sequence Synthetic oligonucleotide
tttgacaagcagaagacggcatc

SEQ ID NO: 320 - DNA, 23 bp, Artificial Sequence
  Description of Artificial Sequence Synthetic oligonucleotide
tttgacaagcagaagaaggcatc

SEQ ID NO: 321 - DNA, 23 bp, Artificial Sequence
  Description of Artificial Sequence Synthetic oligonucleotide
tttgacaagcagaagaacgcatc

SEQ ID NO: 322 - DNA, 23 bp, Artificial Sequence
  Description of Artificial Sequence Synthetic oligonucleotide
tttgacaagcagaagaacggatc

SEQ ID NO: 323 - DNA, 23 bp, Artificial Sequence
  Description of Artificial Sequence Synthetic oligonucleotide
tttgacaagcagaagaacggcac

SEQ ID NO: 324 - DNA, 24 bp, Artificial Sequence
  Description of Artificial Sequence Synthetic oligonucleotide
tgggacaagcagaagaacggcatc

SEQ ID NO: 325 - DNA, 24 bp, Artificial Sequence
  Description of Artificial Sequence Synthetic oligonucleotide
ttgtacaagcagaagaacggcatc

SEQ ID NO: 326 - DNA, 24 bp, Artificial Sequence
  Description of Artificial Sequence Synthetic oligonucleotide
ttttccaagcagaagaacggcatc

SEQ ID NO: 327 - DNA, 24 bp, Artificial Sequence
  Description of Artificial Sequence Synthetic oligonucleotide
tttgcaaagcagaagaacggcatc

SEQ ID NO: 328 - DNA, 24 bp, Artificial Sequence
  Description of Artificial Sequence Synthetic oligonucleotide
tttgaacagcagaagaacggcatc

SEQ ID NO: 329 - DNA, 24 bp, Artificial Sequence
  Description of Artificial Sequence Synthetic oligonucleotide
tttgacccgcagaagaacggcatc

SEQ ID NO: 330 - DNA, 24 bp, Artificial Sequence
  Description of Artificial Sequence Synthetic oligonucleotide
tttgacactcagaagaacggcatc

SEQ ID NO: 331 - DNA, 24 bp, Artificial Sequence
  Description of Artificial Sequence Synthetic oligonucleotide
tttgacaataagaagaacggcatc

SEQ ID NO: 332 - DNA, 24 bp, Artificial Sequence
  Description of Artificial Sequence Synthetic oligonucleotide
tttgacaagacgaagaacggcatc

SEQ ID NO: 333 - DNA, 24 bp, Artificial Sequence
  Description of Artificial Sequence Synthetic oligonucleotide
tttgacaagcctaagaacggcatc

SEQ ID NO: 334 - DNA, 24 bp, Artificial Sequence
  Description of Artificial Sequence Synthetic oligonucleotide
tttgacaagcatcagaacggcatc

SEQ ID NO: 335 - DNA, 24 bp, Artificial Sequence
  Description of Artificial Sequence Synthetic oligonucleotide
tttgacaagcagccgaacggcatc

SEQ ID NO: 336 - DNA, 24 bp, Artificial Sequence
  Description of Artificial Sequence Synthetic oligonucleotide
tttgacaagcagactaacggcatc

SEQ ID NO: 337 - DNA, 24 bp, Artificial Sequence
  Description of Artificial Sequence Synthetic oligonucleotide
tttgacaagcagaatcacggcatc

SEQ ID NO: 338 - DNA, 24 bp, Artificial Sequence
  Description of Artificial Sequence Synthetic oligonucleotide
tttgacaagcagaagcccggcatc

SEQ ID NO: 339 - DNA, 24 bp, Artificial Sequence
  Description of Artificial Sequence Synthetic oligonucleotide
tttgacaagcagaagacaggcatc

SEQ ID NO: 340 - DNA, 24 bp, Artificial Sequence
  Description of Artificial Sequence Synthetic oligonucleotide
tttgacaagcagaagaaatgcatc

SEQ ID NO: 341 - DNA, 24 bp, Artificial Sequence
  Description of Artificial Sequence Synthetic oligonucleotide
tttgacaagcagaagaacttcatc

SEQ ID NO: 342 - DNA, 24 bp, Artificial Sequence
  Description of Artificial Sequence Synthetic oligonucleotide
tttgacaagcagaagaacgtaatc

SEQ ID NO: 343 - DNA, 23 bp, Artificial Sequence
  Description of Artificial Sequence Synthetic oligonucleotide
ttttcatattgttgacgtctcac

SEQ ID NO: 344 - DNA, 23 bp, Artificial Sequence
  Description of Artificial Sequence Synthetic oligonucleotide
ttttcctattgttgacgtctcac

SEQ ID NO: 345 - DNA, 23 bp, Artificial Sequence
  Description of Artificial Sequence Synthetic oligonucleotide
ttttccaattgttgacgtctcac

SEQ ID NO: 346 - DNA, 23 bp, Artificial Sequence
  Description of Artificial Sequence Synthetic oligonucleotide
ttttccatttgttgacgtctcac

SEQ ID NO: 347 - DNA, 23 bp, Artificial Sequence
  Description of Artificial Sequence Synthetic oligonucleotide
ttttccatatgttgacgtctcac

SEQ ID NO: 348 - DNA, 23 bp, Artificial Sequence
  Description of Artificial Sequence Synthetic oligonucleotide
ttttccatattttgacgtctcac

SEQ ID NO: 349 - DNA, 23 bp, Artificial Sequence
  Description of Artificial Sequence Synthetic oligonucleotide
ttttccatattgtgacgtctcac

SEQ ID NO: 350 - DNA, 23 bp, Artificial Sequence
  Description of Artificial Sequence Synthetic oligonucleotide
ttttccatattgttacgtctcac

SEQ ID NO: 351 - DNA, 23 bp, Artificial Sequence
  Description of Artificial Sequence Synthetic oligonucleotide
ttttccatattgttgcgtctcac

SEQ ID NO: 352 - DNA, 23 bp, Artificial Sequence
  Description of Artificial Sequence Synthetic oligonucleotide
ttttccatattgttgagtctcac

SEQ ID NO: 353 - DNA, 23 bp, Artificial Sequence
  Description of Artificial Sequence Synthetic oligonucleotide
ttttccatattgttgactctcac

SEQ ID NO: 354 - DNA, 23 bp, Artificial Sequence
  Description of Artificial Sequence Synthetic oligonucleotide
ttttccatattgttgacgctcac

SEQ ID NO: 355 - DNA, 23 bp, Artificial Sequence
  Description of Artificial Sequence Synthetic oligonucleotide
ttttccatattgttgacgttcac

SEQ ID NO: 356 - DNA, 23 bp, Artificial Sequence
  Description of Artificial Sequence Synthetic oligonucleotide
ttttccatattgttgacgtccac

SEQ ID NO: 357 - DNA, 24 bp, Artificial Sequence
  Description of Artificial Sequence Synthetic oligonucleotide
tggtccatattgttgacgtctcac

SEQ ID NO: 358 - DNA, 24 bp, Artificial Sequence
  Description of Artificial Sequence Synthetic oligonucleotide
ttggccatattgttgacgtctcac

SEQ ID NO: 359 - DNA, 24 bp, Artificial Sequence
  Description of Artificial Sequence Synthetic oligonucleotide
tttgacatattgttgacgtctcac

SEQ ID NO: 360 - DNA, 24 bp, Artificial Sequence
  Description of Artificial Sequence Synthetic oligonucleotide
ttttaaatattgttgacgtctcac

SEQ ID NO: 361 - DNA, 24 bp, Artificial Sequence
  Description of Artificial Sequence Synthetic oligonucleotide
ttttcactattgttgacgtctcac

SEQ ID NO: 362 - DNA, 24 bp, Artificial Sequence
  Description of Artificial Sequence Synthetic oligonucleotide
ttttcccgattgttgacgtctcac

SEQ ID NO: 363 - DNA, 24 bp, Artificial Sequence
  Description of Artificial Sequence Synthetic oligonucleotide
ttttccagcttgttgacgtctcac

SEQ ID NO: 364 - DNA, 24 bp, Artificial Sequence
  Description of Artificial Sequence Synthetic oligonucleotide
ttttccatcgtgttgacgtctcac

SEQ ID NO: 365 - DNA, 24 bp, Artificial Sequence
  Description of Artificial Sequence Synthetic oligonucleotide
ttttccatagggttgacgtctcac

SEQ ID NO: 366 - DNA, 24 bp, Artificial Sequence
  Description of Artificial Sequence Synthetic oligonucleotide
ttttccatatgtttgacgtctcac

SEQ ID NO: 367 - DNA, 24 bp, Artificial Sequence
  Description of Artificial Sequence Synthetic oligonucleotide
ttttccatatttgtgacgtctcac

SEQ ID NO: 368 - DNA, 24 bp, Artificial Sequence
  Description of Artificial Sequence Synthetic oligonucleotide
ttttccatattggggacgtctcac

SEQ ID NO: 369 - DNA, 24 bp, Artificial Sequence
  Description of Artificial Sequence Synthetic oligonucleotide
ttttccatattgtgtacgtctcac

SEQ ID NO: 370 - DNA, 24 bp, Artificial Sequence
  Description of Artificial Sequence Synthetic oligonucleotide
ttttccatattgtttccgtctcac

SEQ ID NO: 371 - DNA, 24 bp, Artificial Sequence
  Description of Artificial Sequence Synthetic oligonucleotide
ttttccatattgttgcagtctcac

SEQ ID NO: 372 - DNA, 24 bp, Artificial Sequence
  Description of Artificial Sequence Synthetic oligonucleotide
ttttccatattgttgaattctcac

SEQ ID NO: 373 - DNA, 24 bp, Artificial Sequence
  Description of Artificial Sequence Synthetic oligonucleotide
ttttccatattgttgactgctcac

SEQ ID NO: 374 - DNA, 24 bp, Artificial Sequence
  Description of Artificial Sequence Synthetic oligonucleotide
ttttccatattgttgacggatcac

SEQ ID NO: 375 - DNA, 24 bp, Artificial Sequence
  Description of Artificial Sequence Synthetic oligonucleotide
ttttccatattgttgacgtagcac

SEQ ID NO: 376 - DNA, 24 bp, Artificial Sequence
  Description of Artificial Sequence Synthetic oligonucleotide
tttaccggcaagctgccattgccc

SEQ ID NO: 377 - DNA, 24 bp, Artificial Sequence
  Description of Artificial Sequence Synthetic oligonucleotide
tttaccggcaagctgcccgtgccc

SEQ ID NO: 378 - DNA, 20 bp, Artificial Sequence
  Description of Artificial Sequence Synthetic oligonucleotide
ccggcaagctgcccgtgccc

SEQ ID NO: 379 - DNA, 24 bp, Artificial Sequence
  Description of Artificial Sequence Synthetic oligonucleotide
tttaccggcaagctgaacgtgccc

SEQ ID NO: 380 - DNA, 20 bp, Artificial Sequence
  Description of Artificial Sequence Synthetic oligonucleotide
ccggcaagctgcccgtgccc

SEQ ID NO: 381 - DNA, 24 bp, Artificial Sequence
  Description of Artificial Sequence Synthetic oligonucleotide
tttaccggcaagctgccattgccc

SEQ ID NO: 382 - DNA, 24 bp, Artificial Sequence
  Description of Artificial Sequence Synthetic oligonucleotide
tttaccggcaagctgcccgtgccc

SEQ ID NO: 383 - DNA, 20 bp, Artificial Sequence
  Description of Artificial Sequence Synthetic oligonucleotide
ccggcaagctgcccgtgccc

SEQ ID NO: 384 - DNA, 24 bp, Artificial Sequence
  Description of Artificial Sequence Synthetic oligonucleotide
tttaccggcaagctgaacgtgccc

SEQ ID NO: 385 - DNA, 20 bp, Artificial Sequence
  Description of Artificial Sequence Synthetic oligonucleotide
ccggcaagctgcccgtgccc

SEQ ID NO: 386 - DNA, 20 bp, Artificial Sequence
  Description of Artificial Sequence Synthetic oligonucleotide
ccggcaagctgaacgtgccc

SEQ ID NO: 387 - DNA, 24 bp, Artificial Sequence
  Description of Artificial Sequence Synthetic oligonucleotide
tttaccggcaagctgcccgtgccc

SEQ ID NO: 388 - DNA, 20 bp, Artificial Sequence
  Description of Artificial Sequence Synthetic oligonucleotide
ccggcaagctgcccgtgccc

SEQ ID NO: 389 - DNA, 20 bp, Artificial Sequence
  Description of Artificial Sequence Synthetic oligonucleotide
ccggcaagctgccattgccc

SEQ ID NO: 390 - DNA, 24 bp, Artificial Sequence
  Description of Artificial Sequence Synthetic oligonucleotide
tttaccggcaagctgcccgtgccc

SEQ ID NO: 391 - DNA, 20 bp, Artificial Sequence
  Description of Artificial Sequence Synthetic oligonucleotide
tttcccttcagctaaaataa

SEQ ID NO: 392 - DNA, 24 bp, Artificial Sequence
  Description of Artificial Sequence Synthetic oligonucleotide
tttatttcccttcagagaaaataa

SEQ ID NO: 393 - DNA, 24 bp, Artificial Sequence
  Description of Artificial Sequence Synthetic oligonucleotide
tttatttcccttcagctaaaataa

SEQ ID NO: 394 - DNA, 20 bp, Artificial Sequence
  Description of Artificial Sequence Synthetic oligonucleotide
ccactcactgctttctcctc

SEQ ID NO: 395 - DNA, 24 bp, Artificial Sequence
  Description of Artificial Sequence Synthetic oligonucleotide
tttcccactcactgcggtctcctc

SEQ ID NO: 396 - DNA, 24 bp, Artificial Sequence
  Description of Artificial Sequence Synthetic oligonucleotide
tttcccactcactgctttctcctc

SEQ ID NO: 397 - DNA, 20 bp, Artificial Sequence
  Description of Artificial Sequence Synthetic oligonucleotide
ccactcactgctttctcctc

SEQ ID NO: 398 - DNA, 24 bp, Artificial Sequence
  Description of Artificial Sequence Synthetic oligonucleotide
tttcccactcactgcggtctcctc

SEQ ID NO: 399 - DNA, 24 bp, Artificial Sequence
  Description of Artificial Sequence Synthetic oligonucleotide
tttcccactcactgctttctcctc

SEQ ID NO: 400 - DNA, 19 bp, Artificial Sequence
  Description of Artificial Sequence Synthetic oligonucleotide
gtcggcatggccccattcg

SEQ ID NO: 401 - DNA, 23 bp, Artificial Sequence
  Description of Artificial Sequence Synthetic oligonucleotide
tttggtcggcatggcaacattcg

SEQ ID NO: 402 - DNA, 23 bp, Artificial Sequence
  Description of Artificial Sequence Synthetic oligonucleotide
tttggtcggcatggccccattcg

SEQ ID NO: 403 - DNA, 22 bp, Artificial Sequence
  Description of Artificial Sequence Synthetic oligonucleotide
ggcacgggcagcttgccggtgg

SEQ ID NO: 404 - DNA, 22 bp, Artificial Sequence
  Description of Artificial Sequence Synthetic oligonucleotide
gggacgggcagcttgccggtgg

SEQ ID NO: 405 - DNA, 22 bp, Artificial Sequence
  Description of Artificial Sequence Synthetic oligonucleotide
gggccgggcagcttgccggtgg

SEQ ID NO: 406 - DNA, 22 bp, Artificial Sequence
  Description of Artificial Sequence Synthetic oligonucleotide
gggcagggcagcttgccggtgg

SEQ ID NO: 407 - DNA, 22 bp, Artificial Sequence
  Description of Artificial Sequence Synthetic oligonucleotide
gggcacggcagcttgccggtgg

SEQ ID NO: 408 - DNA, 22 bp, Artificial Sequence
  Description of Artificial Sequence Synthetic oligonucleotide
gggcacgggagcttgccggtgg

SEQ ID NO: 409 - DNA, 22 bp, Artificial Sequence
  Description of Artificial Sequence Synthetic oligonucleotide
gggcacgggcgcttgccggtgg

SEQ ID NO: 410 - DNA, 22 bp, Artificial Sequence
  Description of Artificial Sequence Synthetic oligonucleotide
gggcacgggcacttgccggtgg

SEQ ID NO: 411 - DNA, 22 bp, Artificial Sequence
  Description of Artificial Sequence Synthetic oligonucleotide
gggcacgggcagttgccggtgg

SEQ ID NO: 412 - DNA, 22 bp, Artificial Sequence
  Description of Artificial Sequence Synthetic oligonucleotide
gggcacgggcagctgccggtgg

SEQ ID NO: 413 - DNA, 22 bp, Artificial Sequence
  Description of Artificial Sequence Synthetic oligonucleotide
gggcacgggcagcttccggtgg

SEQ ID NO: 414 - DNA, 22 bp, Artificial Sequence
  Description of Artificial Sequence Synthetic oligonucleotide
gggcacgggcagcttgcggtgg

SEQ ID NO: 415 - DNA, 22 bp, Artificial Sequence
  Description of Artificial Sequence Synthetic oligonucleotide
gggcacgggcagcttgccgtgg

SEQ ID NO: 416 - DNA, 23 bp, Artificial Sequence
  Description of Artificial Sequence Synthetic oligonucleotide
gggcacgggcagcttgcccctgg

SEQ ID NO: 417 - DNA, 23 bp, Artificial Sequence
  Description of Artificial Sequence Synthetic oligonucleotide
gggcacgggcagcttgggggtgg

SEQ ID NO: 418 - DNA, 23 bp, Artificial Sequence
  Description of Artificial Sequence Synthetic oligonucleotide
gggcacgggcagctacccggtgg

SEQ ID NO: 419 - DNA, 23 bp, Artificial Sequence
  Description of Artificial Sequence Synthetic oligonucleotide
gggcacgggcaggatgccggtgg

SEQ ID NO: 420 - DNA, 23 bp, Artificial Sequence
  Description of Artificial Sequence Synthetic oligonucleotide
gggcacgggctccttgccggtgg

SEQ ID NO: 421 - DNA, 23 bp, Artificial Sequence
  Description of Artificial Sequence Synthetic oligonucleotide
gggcacccgcagcttgccggtgg

SEQ ID NO: 422 - DNA, 23 bp, Artificial Sequence
  Description of Artificial Sequence Synthetic oligonucleotide
gggcacggcgagcttgccggtgg

SEQ ID NO: 423 - DNA, 23 bp, Artificial Sequence
  Description of Artificial Sequence Synthetic oligonucleotide
gggctggggcagcttgccggtgg

SEQ ID NO: 424 - DNA, 22 bp, Artificial Sequence
  Description of Artificial Sequence Synthetic oligonucleotide
atgccgttcttctgcttgtcgg

SEQ ID NO: 425 - DNA, 22 bp, Artificial Sequence
  Description of Artificial Sequence Synthetic oligonucleotide
gtgccgttcttctgcttgtcgg

SEQ ID NO: 426 - DNA, 22 bp, Artificial Sequence
  Description of Artificial Sequence Synthetic oligonucleotide
gagccgttcttctgcttgtcgg

SEQ ID NO: 427 - DNA, 22 bp, Artificial Sequence
  Description of Artificial Sequence Synthetic oligonucleotide
gatccgttcttctgcttgtcgg

SEQ ID NO: 428 - DNA, 22 bp, Artificial Sequence
  Description of Artificial Sequence Synthetic oligonucleotide
gatgcgttcttctgcttgtcgg

SEQ ID NO: 429 - DNA, 22 bp, Artificial Sequence
  Description of Artificial Sequence Synthetic oligonucleotide
gatgccttcttctgcttgtcgg

SEQ ID NO: 430 - DNA, 22 bp, Artificial Sequence
  Description of Artificial Sequence Synthetic oligonucleotide
gatgccgtcttctgcttgtcgg

SEQ ID NO: 431 - DNA, 22 bp, Artificial Sequence
  Description of Artificial Sequence Synthetic oligonucleotide
gatgccgttttctgcttgtcgg

SEQ ID NO: 432 - DNA, 22 bp, Artificial Sequence
  Description of Artificial Sequence Synthetic oligonucleotide
gatgccgttctctgcttgtcgg

SEQ ID NO: 433 - DNA, 22 bp, Artificial Sequence
  Description of Artificial Sequence Synthetic oligonucleotide
gatgccgttctttgcttgtcgg

SEQ ID NO: 434 - DNA, 22 bp, Artificial Sequence
  Description of Artificial Sequence Synthetic oligonucleotide
gatgccgttcttcgcttgtcgg

SEQ ID NO: 435 - DNA, 22 bp, Artificial Sequence
  Description of Artificial Sequence Synthetic oligonucleotide
gatgccgttcttctcttgtcgg

SEQ ID NO: 436 - DNA, 22 bp, Artificial Sequence
  Description of Artificial Sequence Synthetic oligonucleotide
gatgccgttcttctgttgtcgg

SEQ ID NO: 437 - DNA, 22 bp, Artificial Sequence
  Description of Artificial Sequence Synthetic oligonucleotide
gatgccgttcttctgctgtcgg

SEQ ID NO: 438 - DNA, 22 bp, Artificial Sequence
  Description of Artificial Sequence Synthetic oligonucleotide
gatgccgttcttctgctttcgg

SEQ ID NO: 439 - DNA, 22 bp, Artificial Sequence
  Description of Artificial Sequence Synthetic oligonucleotide
gatgccgttcttctgcttgcgg

SEQ ID NO: 440 - DNA, 23 bp, Artificial Sequence
  Description of Artificial Sequence Synthetic oligonucleotide
gatggggttcttctgcttgtcgg

SEQ ID NO: 441 - DNA, 23 bp, Artificial Sequence
  Description of Artificial Sequence Synthetic oligonucleotide
gatgcccatcttctgcttgtcgg

SEQ ID NO: 442 - DNA, 23 bp, Artificial Sequence
  Description of Artificial Sequence Synthetic oligonucleotide
gatgccgtagttctgcttgtcgg

SEQ ID NO: 443 - DNA, 23 bp, Artificial Sequence
  Description of Artificial Sequence Synthetic oligonucleotide
gatgccgttcaactgcttgtcgg

SEQ ID NO: 444 - DNA, 23 bp, Artificial Sequence
  Description of Artificial Sequence Synthetic oligonucleotide
gatgccgttcttgagcttgtcgg

SEQ ID NO: 445 - DNA, 23 bp, Artificial Sequence
  Description of Artificial Sequence Synthetic oligonucleotide
gatgccgttcttctcgttgtcgg

SEQ ID NO: 446 - DNA, 23 bp, Artificial Sequence
  Description of Artificial Sequence Synthetic oligonucleotide
gatgccgttcttctgcaagtcgg

SEQ ID NO: 447 - DNA, 23 bp, Artificial Sequence
  Description of Artificial Sequence Synthetic oligonucleotide
gatgccgttcttctgcttcacgg

SEQ ID NO: 448 - DNA, 22 bp, Artificial Sequence
  Description of Artificial Sequence Synthetic oligonucleotide
tgagacgtcaacaatatggagg

SEQ ID NO: 449 - DNA, 22 bp, Artificial Sequence
  Description of Artificial Sequence Synthetic oligonucleotide
ggagacgtcaacaatatggagg

SEQ ID NO: 450 - DNA, 22 bp, Artificial Sequence
  Description of Artificial Sequence Synthetic oligonucleotide
gtagacgtcaacaatatggagg

SEQ ID NO: 451 - DNA, 22 bp, Artificial Sequence
  Description of Artificial Sequence Synthetic oligonucleotide
gtggacgtcaacaatatggagg

SEQ ID NO: 452 - DNA, 22 bp, Artificial Sequence
  Description of Artificial Sequence Synthetic oligonucleotide
gtgaacgtcaacaatatggagg

SEQ ID NO: 453 - DNA, 22 bp, Artificial Sequence
  Description of Artificial Sequence Synthetic oligonucleotide
gtgagcgtcaacaatatggagg

SEQ ID NO: 454 - DNA, 22 bp, Artificial Sequence
  Description of Artificial Sequence Synthetic oligonucleotide
gtgagagtcaacaatatggagg

SEQ ID NO: 455 - DNA, 22 bp, Artificial Sequence
  Description of Artificial Sequence Synthetic oligonucleotide
gtgagactcaacaatatggagg

SEQ ID NO: 456 - DNA, 22 bp, Artificial Sequence
  Description of Artificial Sequence Synthetic oligonucleotide
gtgagacgcaacaatatggagg

SEQ ID NO: 457 - DNA, 22 bp, Artificial Sequence
  Description of Artificial Sequence Synthetic oligonucleotide
gtgagacgtaacaatatggagg

SEQ ID NO: 458 - DNA, 22 bp, Artificial Sequence
  Description of Artificial Sequence Synthetic oligonucleotide
gtgagacgtcacaatatggagg

SEQ ID NO: 459 - DNA, 22 bp, Artificial Sequence
  Description of Artificial Sequence Synthetic oligonucleotide
gtgagacgtcaaaatatggagg

SEQ ID NO: 460 - DNA, 22 bp, Artificial Sequence
  Description of Artificial Sequence Synthetic oligonucleotide
gtgagacgtcaacatatggagg

SEQ ID NO: 461 - DNA, 22 bp, Artificial Sequence
  Description of Artificial Sequence Synthetic oligonucleotide
gtgagacgtcaacaaatggagg

SEQ ID NO: 462 - DNA, 22 bp, Artificial Sequence
  Description of Artificial Sequence Synthetic oligonucleotide
gtgagacgtcaacaattggagg

SEQ ID NO: 463 - DNA, 22 bp, Artificial Sequence
  Description of Artificial Sequence Synthetic oligonucleotide
gtgagacgtcaacaataggagg

SEQ ID NO: 464 - DNA, 22 bp, Artificial Sequence
  Description of Artificial Sequence Synthetic oligonucleotide
gtgagacgtcaacaatatgagg

SEQ ID NO: 465 - DNA, 23 bp, Artificial Sequence
  Description of Artificial Sequence Synthetic oligonucleotide
gtgactcgtcaacaatatggagg

SEQ ID NO: 466 - DNA, 23 bp, Artificial Sequence
  Description of Artificial Sequence Synthetic oligonucleotide
gtgagagctcaacaatatggagg

SEQ ID NO: 467 - DNA, 23 bp, Artificial Sequence
  Description of Artificial Sequence Synthetic oligonucleotide
gtgagacgagaacaatatggagg

SEQ ID NO: 468 - DNA, 23 bp, Artificial Sequence
  Description of Artificial Sequence Synthetic oligonucleotide
gtgagacgtcttcaatatggagg

SEQ ID NO: 469 - DNA, 23 bp, Artificial Sequence
  Description of Artificial Sequence Synthetic oligonucleotide
gtgagacgtcaagtatatggagg

SEQ ID NO: 470 - DNA, 23 bp, Artificial Sequence
  Description of Artificial Sequence Synthetic oligonucleotide
gtgagacgtcaacataatggagg

SEQ ID NO: 471 - DNA, 23 bp, Artificial Sequence
  Description of Artificial Sequence Synthetic oligonucleotide
gtgagacgtcaacaattaggagg

SEQ ID NO: 472 - DNA, 23 bp, Artificial Sequence
  Description of Artificial Sequence Synthetic oligonucleotide
gtgagacgtcaacaatatccagg

SEQ ID NO: 473 - DNA, 22 bp, Artificial Sequence
  Description of Artificial Sequence Synthetic oligonucleotide
caccaccgtcgccggcatttgg

SEQ ID NO: 474 - DNA, 22 bp, Artificial Sequence
  Description of Artificial Sequence Synthetic oligonucleotide
gaccaccgtcgccggcatttgg

SEQ ID NO: 475 - DNA, 22 bp, Artificial Sequence
  Description of Artificial Sequence Synthetic oligonucleotide
gcccaccgtcgccggcatttgg

SEQ ID NO: 476 - DNA, 22 bp, Artificial Sequence
  Description of Artificial Sequence Synthetic oligonucleotide
gcacaccgtcgccggcatttgg

SEQ ID NO: 477 - DNA, 22 bp, Artificial Sequence
  Description of Artificial Sequence Synthetic oligonucleotide
gcaccccgtcgccggcatttgg

SEQ ID NO: 478 - DNA, 22 bp, Artificial Sequence
  Description of Artificial Sequence Synthetic oligonucleotide
gcaccacgtcgccggcatttgg

SEQ ID NO: 479 - DNA, 22 bp, Artificial Sequence
  Description of Artificial Sequence Synthetic oligonucleotide
gcaccacctcgccggcatttgg

SEQ ID NO: 480 - DNA, 22 bp, Artificial Sequence
  Description of Artificial Sequence Synthetic oligonucleotide
gcaccaccgcgccggcatttgg

SEQ ID NO: 481 - DNA, 22 bp, Artificial Sequence
  Description of Artificial Sequence Synthetic oligonucleotide
gcaccaccgtgccggcatttgg

SEQ ID NO: 482 - DNA, 22 bp, Artificial Sequence
  Description of Artificial Sequence Synthetic oligonucleotide
gcaccaccgtcccggcatttgg

SEQ ID NO: 483 - DNA, 22 bp, Artificial Sequence
  Description of Artificial Sequence Synthetic oligonucleotide
gcaccaccgtcgcggcatttgg

SEQ ID NO: 484 - DNA, 22 bp, Artificial Sequence
  Description of Artificial Sequence Synthetic oligonucleotide
gcaccaccgtcgccgcatttgg

SEQ ID NO: 485 - DNA, 22 bp, Artificial Sequence
  Description of Artificial Sequence Synthetic oligonucleotide
gcaccaccgtcgccggatttgg

SEQ ID NO: 486 - DNA, 22 bp, Artificial Sequence
  Description of Artificial Sequence Synthetic oligonucleotide
gcaccaccgtcgccggctttgg

SEQ ID NO: 487 - DNA, 22 bp, Artificial Sequence
  Description of Artificial Sequence Synthetic oligonucleotide
gcaccaccgtcgccggcattgg

SEQ ID NO: 488 - DNA, 23 bp, Artificial Sequence
  Description of Artificial Sequence Synthetic oligonucleotide
cgaccaccgtcgccggcatttgg

SEQ ID NO: 489 - DNA, 23 bp, Artificial Sequence
  Description of Artificial Sequence Synthetic oligonucleotide
gctgcaccgtcgccggcatttgg

SEQ ID NO: 490 - DNA, 23 bp, Artificial Sequence
  Description of Artificial Sequence Synthetic oligonucleotide
gcacgtccgtcgccggcatttgg

SEQ ID NO: 491 - DNA, 23 bp, Artificial Sequence
  Description of Artificial Sequence Synthetic oligonucleotide
gcaccagggtcgccggcatttgg

SEQ ID NO: 492 - DNA, 23 bp, Artificial Sequence
  Description of Artificial Sequence Synthetic oligonucleotide
gcaccacccacgccggcatttgg

SEQ ID NO: 493 - DNA, 23 bp, Artificial Sequence
  Description of Artificial Sequence Synthetic oligonucleotide
gcaccaccgtgcccggcatttgg

SEQ ID NO: 494 - DNA, 23 bp, Artificial Sequence
  Description of Artificial Sequence Synthetic oligonucleotide
gcaccaccgtcgggggcatttgg

SEQ ID NO: 495 - DNA, 23 bp, Artificial Sequence
  Description of Artificial Sequence Synthetic oligonucleotide
gcaccaccgtcgcccccatttgg

SEQ ID NO: 496 - DNA, 23 bp, Artificial Sequence
  Description of Artificial Sequence Synthetic oligonucleotide
gcaccaccgtcgccgggttttgg

SEQ ID NO: 497 - DNA, 23 bp, Artificial Sequence
  Description of Artificial Sequence Synthetic oligonucleotide
gcaccaccgtcgccggcaaatgg

SEQ ID NO: 498 - DNA, 35 bp, Artificial Sequence
  Description of Artificial Sequence Synthetic oligonucleotide
  modified_base (1)..(6): a, c, t, g, unknown or other
  modified_base (30)..(35): a, c, t, g, unknown or other
nnnnnngcaccaccgtcgccggcatttggnnnnnn

SEQ ID NO: 499 - DNA, 23 bp, Artificial Sequence
  Description of Artificial Sequence Synthetic oligonucleotide
gcaccaccgtcgccggcatttgg

SEQ ID NO: 500 - DNA, 19 bp, Artificial Sequence
  Description of Artificial Sequence Synthetic oligonucleotide
gcgccaccgtcgccgcatt

SEQ ID NO: 501 - DNA, 19 bp, Artificial Sequence
  Description of Artificial Sequence Synthetic oligonucleotide
gatgccgttctttgcttgt

SEQ ID NO: 502 - DNA, 19 bp, Artificial Sequence
  Description of Artificial Sequence Synthetic oligonucleotide
gatggcgtcttctgctttt

SEQ ID NO: 503 - DNA, 23 bp, Artificial Sequence
  Description of Artificial Sequence Synthetic oligonucleotide
gatgccgttcttctgcttgtcgg

SEQ ID NO: 504 - DNA, 23 bp, Artificial Sequence
  Description of Artificial Sequence Synthetic oligonucleotide
ccaaatgccggcgacgggggtgc

SEQ ID NO: 505 - DNA, 20 bp, Artificial Sequence
  Description of Artificial Sequence Synthetic oligonucleotide
gatgccgttcttctgcttgt

SEQ ID NO: 506 - DNA, 20 bp, Artificial Sequence
  Description of Artificial Sequence Synthetic oligonucleotide
gcaccaccgtcgccggcatt

SEQ ID NO: 507 - DNA, 23 bp, Artificial Sequence
  Description of Artificial Sequence Synthetic oligonucleotide
gtgagacgtcaacaatatggagg

SEQ ID NO: 508 - DNA, 23 bp, Artificial Sequence
  Description of Artificial Sequence Synthetic oligonucleotide
cctccatattgttgacgtctcac

SEQ ID NO: 509 - DNA, 20 bp, Artificial Sequence
  Description of Artificial Sequence Synthetic oligonucleotide
gcaccaccgtcgccggcatt

SEQ ID NO: 510 - DNA, 22 bp, Artificial Sequence
  Description of Artificial Sequence Synthetic oligonucleotide
caccaccgtcgccggcatttgg

SEQ ID NO: 511 - DNA, 22 bp, Artificial Sequence
  Description of Artificial Sequence Synthetic oligonucleotide
gaccaccgtcgccggcatttgg

SEQ ID NO: 512 - DNA, 22 bp, Artificial Sequence
  Description of Artificial Sequence Synthetic oligonucleotide
gcccaccgtcgccggcatttgg

SEQ ID NO: 513 - DNA, 22 bp, Artificial Sequence
  Description of Artificial Sequence Synthetic oligonucleotide
gcacaccgtcgccggcatttgg

SEQ ID NO: 514 - DNA, 22 bp, Artificial Sequence
  Description of Artificial Sequence Synthetic oligonucleotide
gcaccccgtcgccggcatttgg

SEQ ID NO: 515 - DNA, 22 bp, Artificial Sequence
  Description of Artificial Sequence Synthetic oligonucleotide
gcaccacgtcgccggcatttgg

SEQ ID NO: 516 - DNA, 22 bp, Artificial Sequence
  Description of Artificial Sequence Synthetic oligonucleotide
gcaccacctcgccggcatttgg

SEQ ID NO: 517 - DNA, 22 bp, Artificial Sequence
  Description of Artificial Sequence Synthetic oligonucleotide
gcaccaccgcgccggcatttgg

SEQ ID NO: 518 - DNA, 22 bp, Artificial Sequence
  Description of Artificial Sequence Synthetic oligonucleotide
gcaccaccgtgccggcatttgg

SEQ ID NO: 519 - DNA, 22 bp, Artificial Sequence
  Description of Artificial Sequence Synthetic oligonucleotide
gcaccaccgtcccggcatttgg

SEQ ID NO: 520 - DNA, 22 bp, Artificial Sequence
  Description of Artificial Sequence Synthetic oligonucleotide
gcaccaccgtcgcggcatttgg

SEQ ID NO: 521 - DNA, 22 bp, Artificial Sequence
  Description of Artificial Sequence Synthetic oligonucleotide
gcaccaccgtcgccgcatttgg

SEQ ID NO: 522 - DNA, 22 bp, Artificial Sequence
  Description of Artificial Sequence Synthetic oligonucleotide
gcaccaccgtcgccggatttgg

SEQ ID NO: 523 - DNA, 22 bp, Artificial Sequence
  Description of Artificial Sequence Synthetic oligonucleotide
gcaccaccgtcgccggctttgg

SEQ ID NO: 524 - DNA, 22 bp, Artificial Sequence
  Description of Artificial Sequence Synthetic oligonucleotide
gcaccaccgtcgccggcattgg

SEQ ID NO: 525 - DNA, 23 bp, Artificial Sequence
  Description of Artificial Sequence Synthetic oligonucleotide
cgaccaccgtcgccggcatttgg

SEQ ID NO: 526 - DNA, 23 bp, Artificial Sequence
  Description of Artificial Sequence Synthetic oligonucleotide
gctgcaccgtcgccggcatttgg

SEQ ID NO: 527 - DNA, 23 bp, Artificial Sequence
  Description of Artificial Sequence Synthetic oligonucleotide
gcacgtccgtcgccggcatttgg

SEQ ID NO: 528 - DNA, 23 bp, Artificial Sequence
  Description of Artificial Sequence Synthetic oligonucleotide
gcaccagggtcgccggcatttgg

SEQ ID NO: 529 - DNA, 23 bp, Artificial Sequence
  Description of Artificial Sequence Synthetic oligonucleotide
gcaccacccacgccggcatttgg

SEQ ID NO: 530 - DNA, 23 bp, Artificial Sequence
  Description of Artificial Sequence Synthetic oligonucleotide
gcaccaccgtgcccggcatttgg

SEQ ID NO: 531 - DNA, 23 bp, Artificial Sequence
  Description of Artificial Sequence Synthetic oligonucleotide
gcaccaccgtcgggggcatttgg

SEQ ID NO: 532 - DNA, 23 bp, Artificial Sequence
  Description of Artificial Sequence Synthetic oligonucleotide
gcaccaccgtcgcccccatttgg

SEQ ID NO: 533 - DNA, 23 bp, Artificial Sequence
  Description of Artificial Sequence Synthetic oligonucleotide
gcaccaccgtcgccgggttttgg

SEQ ID NO: 534 - DNA, 23 bp, Artificial Sequence
  Description of Artificial Sequence Synthetic oligonucleotide
gcaccaccgtcgccggcaaatgg